(19) United States
(12) Patent Application Publication
(10) Pub. No.: US 2019/0125735 A1
(43) Pub. Date: May 2, 2019

(54) INHIBITION OF P38 MAPK FOR THE TREATMENT OF CANCER

(71) Applicant: Board of Regents, The University of Texas System, Austin, TX (US)

(72) Inventors: Sendurai A. MANI, Houston, TX (US); Steven J. WERDEN, Pearland, TX (US); Anurag N. PARANJAPE, Bethesda, MD (US); Rama SOUNDARARAJAN, Houston, TX (US); Natalia SPHYRIS, Edinburgh (GB); Xiaoping SUN, Houston, TX (US); Saradhi MALLAMPATI, Houston, TX (US)

(21) Appl. No.: 16/064,517

(22) PCT Filed: Dec. 28, 2016
(86) PCT No.: PCT/US16/68813

§ 371 Date: Jun. 21, 2018

Related U.S. Application Data
(60) Provisional application No. 62/272,394, filed on Dec. 29, 2015.
(60) Provisional application No. 62/272,508, filed on Dec. 29, 2015.

(51) Int. Cl. A61K 31/4439; A61P 35/00; A61P 35/04; A61P 35/02; A61K 31/4164; A61K 31/337; A61K 45/06; A61K 31/573; A61K 31/506

(52) U.S. Cl. CPC A61K 31/4439; A61P 35/00; A61P 35/04; A61P 35/02; A61K 31/506; A61K 31/337; A61K 45/06; A61K 31/573; A61K 31/4164

(57) ABSTRACT

Methods are provided for treating cancer patients who have elevated expression of FOXC2. In certain aspects, patients having elevated FOXC2 are treated with an anti-cancer therapy in conjunction with a p38 MAPK inhibitor. Methods of identifying cancer patients to be treated with p38 MAPK inhibitors are also provided. Further provided herein are methods for treating and/or preventing breast cancer metastasis by the administration of a p38 MAPK inhibitor. In certain aspects, patients are treated with an anti-cancer therapy in conjunction with a p38 MAPK inhibitor. In addition, methods are provided for the treatment of BCR-ABL positive cancers, such as acute lymphoblastic leukemia, by the administration of a p38 MAPK inhibitor and/or a glucocorticoid inhibitor in combination with a tyrosine kinase inhibitor.

The present application claims the priority benefit of U.S. Provisional Applications Ser. No. 62/272,394 and 62/272,508, filed Dec. 29, 2015, the entire contents of both applications being hereby incorporated by reference.

The invention was made with government support under Grant No. 5RO1CA155243 awarded by the National Institute of Health and Grant No. RP-130485 awarded by the Cancer Prevention and Research Institute of Texas. The government has certain rights in the invention.

### BACKGROUND OF THE INVENTION

### 1. Field of the Invention

The present invention relates generally to the fields of molecular biology and medicine. More particularly, it concerns compositions and methods of treating cancer, such as prostate cancer, breast cancer, and leukemia.

### 2. Description of Related Art

Prostate Cancer

Prostate cancer (PCa) progression to metastatic disease accounts for >10% of all cancer-related deaths in men. Androgen deprivation therapy (ADT) remains the principal treatment for PCa. While this results in initial tumor regression, the majority of these patients become noncompliant to this line of treatment, owing to the emergence of androgen-independent mechanisms promoting tumor cell growth. Moreover, although most initially diagnosed PCas are acinar adenocarcinomas that display elevated expression of the androgen receptor (AR) and its target gene prostate-specific antigen (PSA), a substantial proportion of patients present atypical clinical features, and are characterized by the blatant absence of AR and PSA, but instead, display immunoreactivity to neuroendocrine (NE) differentiation markers such as chromogranin A, synaptophysin, CD56 and neuron-specific enolase (NSE). While these AR/PSA-negative neuroendocrine prostate cancers (NEPC) (or small-cell prostate carcinomas) are rare at the time of initial diagnosis, they however, account for 10-30% of advanced recurrent castration-resistant prostate cancers (CRPC, high-grade Gleason) following ADT (Aggarwal et al., 2014). These variant “AR-negative PCa” or NEPCs are extremely aggressive, androgen-independent, metastatic and therapy-resistant, with their 5-year overall survival being dismal at 12.6%, which categorizes them as the most deadly subset of all PCa (Parimi et al., 2014). Currently, there are no targeted therapies available for this class of patients, and their AR-negativity presents a major therapeutic challenge.

It has long been recognized that androgens and AR exhibit key tumor suppressive effects in the prostate. Genetic ablation of AR in prostate epithelial cells has actually been demonstrated to promote the development of invasive prostate tumors (Niu et al., 2008), while targeting AR with siRNA has been shown to promote metastasis through enhanced macrophage recruitment via STAT3 activation (Izumi et al., 2013). Moreover, AR expression is significantly reduced in metastatic hormone-resistant PCa (Davis et al., 2006) while AR signaling was found to be severely attenuated in some advanced PCa (Tomlins et al., 2007). Collectively, this argues that restoration of AR and AR signaling could indeed have beneficial effects for the select class of PCa patients (such as those diagnosed with NEPC/small-cell prostate carcinomas, or even advanced adenocarcinomas displaying NE differentiation following ADT) that feature loss of AR or its downstream molecular targets.

Emerging evidence implicates the existence of a subpopulation of androgen-insensitive stem-like cells in prostate tumors (PCaSCs) that may potentially aid in tumor recurrence, metastatic progression and therapy-resistance (Castillo et al., 2014). However, the origin of these cells and the molecular factors governing their stem-like behavior are still poorly understood, although there have been suggestions that PCaSCs may be NE in nature (and hence AR/PSA^{-/lo}) (Santoni et al., 2014). In a recent report aimed at characterizing the cancer stem-cell (CSC) pool from PCa explants, drug-resistant sphere cultures were found to be particularly enriched for cytokeratin (Paranjape et al., 2014), suggesting their epithelial origin. Further, elevated expression of Zeb1, an epithelial-mesenchymal-transition (EMT) transcription factor associated with stem-like properties, in androgen-independent PCa cells as well as in prostate tumors of castrated PTEN conditional knockout mice, advocates that PCaSCs are possibly products of EMT (Li et al., 2014).

EMT refers to a complex cellular reprogramming process that facilitates the conversion of differentiated epithelial cells into loosely organized, highly migratory and invasive mesenchymal cells. Although there are multiple suggestions that deviant activation of EMT pathways may facilitate the development of PCa, and possibly, aid in its progression to the advanced therapy-resistant state (Sun et al., 2012), the precise molecular mechanisms that dictate the EMT/CSC-mediated shift to altered AR signaling as well as to androgen-independence, and the source of stem-cells in PCa progression, remain largely undefined.

Employing a PSA promoter-driven lentiviral EGFP reporter system, it has previously demonstrated that in both primary prostate cancer tissues, and in established PCa cell lines, the PSA^{-/lo} cells represent a functionally unique subpopulation that is selectively enriched for cells characteristic of castration-resistant PCaSC (Qin et al., 2012). This undifferentiated pool of cells expresses classical PCaSC markers (ALDH, CD44, α2β1-integrin), and undergoes asymmetric cell division to generate the PSA⁺/differentiated counterpart of prostate epithelial cells. Further, these cells are endowed with elevated clonogenic potential and tumor-propagating capacity, thereby highlighting the potential clinical benefit of effectively targeting this subpopulation of PCa cells. However, there is a lack of therapeutic methods to eliminate the source/generation of these prostate cancer stem-like cells, the tumor cell subpopulation that critically determines tumor initiation, recurrence, castration-resistance and metastatic progression.

Breast Cancer

More than 90% of cancer-related deaths are attributed to metastases rather than primary tumors (Gupta et al., 2006). In carcinomas, metastatic competence is contingent upon the aberrant activation of a latent embryonic program, termed the epithelial-mesenchymal transition (EMT) (Tsai et al., 2013). EMT is a complex process entailing reprogramming of differentiated epithelial cells towards a mesenchymal phenotype underscored by loss of E-cadherin, reorganization of the actin cytoskeleton, acquisition of mesenchymal markers, and enhanced migratory and invasive potential. Moreover, the induction of EMT in tumor cells confers self-renewal capabilities (Mani et al., 2008). Cumulatively, these properties lead to the de novo generation of metastasis-competent cancer stem cells (CSCs) that can navigate/complete the metastatic cascade and seed new tumor colonies at distal sites.

The Forkhead transcription factor FOXC2 was recently identified as a key downstream effector of multiple EMT programs, independent of the nature of the EMT-inducing stimuli (Mani et al., 2007). In addition, it was found that FOXC2 is necessary and sufficient for the acquisition of CSC properties, chemotherapy resistance and metastatic competence following EMT induction (Hollier et al., 2013). Importantly, FOXC2 expression is elevated in metastasis-prone basal-like and claudin-low CSC-enriched breast cancers, as well as in residual tumor cells isolated from breast cancer patients treated with conventional therapies, which display mesenchymal and stem cell features. Collectively, these findings underscore the clinical relevance of FOXC2 as a potential therapeutic target for metastatic and therapy-resistant breast cancers. However, translating these findings into an effective therapeutic modality is problematic, since FOXC2 is a transcription factor, which—from a pharmacological standpoint—hinders rational drug design. Therefore, the identification of druggable upstream regulators of FOXC2 function may hold the key to developing effective therapies against metastatic breast cancers. However, a druggable upstream kinase that mediates FOXC2 phosphorylation and governs its pleiotropic roles during metastatic progression has yet to be identified.

Leukemia

BCR-ABL, a key oncogene in BCR-ABL-positive (BCR-ABL+; also known as Philadelphia chromosome-positive) B-cell acute lymphoblastic leukemia (ALL), encodes an oncogenic fusion protein with sustained high tyrosine kinase activity. In BCR-ABL+ ALL, different chromosomal breakpoints produce BCR-ABL isoforms with different molecular weights. The p190 BCR-ABL isoform is responsible for approximately 30% of ALL cases and predicts unfavorable prognosis in both adults and children.

Before the introduction of tyrosine kinase inhibitors (TKIs), patients with BCR-ABL+ ALL were treated with chemotherapy but had poor outcomes. Allogeneic stem cell transplantation was offered to all patients in first complete remission. However, stem cell transplantation is associated with toxicity and is limited by the availability of suitable donors. Including imatinib, the prototype of TKIs, in first-line therapy has revolutionized the treatment of BCR-ABL+ ALL, with outcomes comparable to that of stem cell transplantation but with much lower toxicity. Unfortunately, imatinib resistance has become a major challenge. Genetic mutations in the imatinib binding domain of BCR-ABL or various mechanisms independent of genetic mutations promote resistance to imatinib and subsequently cause disease relapse. Nongenetic mechanisms that contribute to the origin of imatinib resistance arise very rapidly and might cause mutation-mediated resistance.

Recently, a novel nongenetic mechanism of drug resistance in BCR-ABL+ ALL was demonstrated, i.e., imatinib-induced mesenchymal stem/stromal cell (MSC)-mediated resistance. It was found that imatinib and other TKIs act like a double-edged sword: on one hand, they kill bulk leukemic cells and are indispensable in treating BCR-ABL+ leukemia, including BCR-ABL+ ALL (on-target effects); on the other hand, they induce structural and functional changes in MSCs and enable MSCs to provide alternative survival signals to leukemic cells (off-target effects). Inhibition of BCR-ABL signaling and activation of alternative survival signaling drive leukemic cells to switch signaling for survival. Imatinib-induced MSC-mediated protection starts early in the course of treatment, which allows leukemic cells to develop additional types of resistance, including those associated with BCR-ABL gene mutations. Thus, there is an unmet need to develop therapies for BCR-ABL positive leukemia.

### SUMMARY OF THE INVENTION

Embodiments of the present disclosure provide methods and compositions for treating cancer in a subject. In a first embodiment, there is provided a method of treating cancer in a subject comprising administering to the subject: (a) a p38 MAPK inhibitor; and (b) an anti-cancer therapy, in an amount effective to treat, wherein the subject is identified as having cancer cells that express an elevated level of FOXC2 relative to a reference level. In some aspects, the subject is a human subject.

In certain aspects, treating comprises inhibiting the growth of primary tumor cells, inhibiting the formation of metastases, inhibiting the growth of metastases, killing circulating cancer cells, inhibiting the growth and/or survival of cancer stem cells, inducing remission, extending remission, or inhibiting recurrence. In some aspects, treating comprises inhibiting the growth and/or survival of cancer stem cells.

In some aspects, the cancer stem cells have decreased expression of N-cadherin, collagen type III-α1, fibronectin, vimentin, Slug, Zeb1, or FOXC2 relative to expression prior to administration of the p38 MAPK inhibitor and the anti-cancer therapy.

In certain aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. In particular aspects, the cancer is prostate cancer. In some aspects, the prostate cancer is androgen-independent. In certain aspects, the prostate cancer is castration-resistant.

In some aspects, the subject has a decreased number of cancer stem cells relative to prior to administration of the p38 MAPK inhibitor and anti-cancer therapy. In certain aspects, the cancer stem cells express one or more markers selected from a group consisting of ALDH, CD44, α2β1-integrin, Bmi1, and Sox2. In some aspects, the cancer stem cells do not express androgen receptor and/or prostate-specific antigen (PSA).

In certain aspects, the anti-cancer therapy is chemotherapy, radiotherapy, gene therapy, surgery, hormonal therapy, anti-angiogenic therapy or cytokine therapy. In some aspects, the hormonal therapy is an androgen-receptor inhibitor. In certain aspects, the androgen-receptor inhibitor is Enzalutamide. In one specific aspect, the chemotherapy is Docetaxel.

In some aspects, the p38 MAPK inhibitor is SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745, DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804. In particular aspects, the p38 MAPK inhibitor is SB 203580. In some aspects, the p38 MAPK inhibitor is SB 203580 and the anti-cancer therapy is Enzalutamide. In other aspects, the p38 MAPK inhibitor is SB 203580 and the anti-cancer therapy is Docetaxel.

In certain aspects, the anti-cancer therapy and/or p38 MAPK inhibitor are administered intravenously, intraperitoneally, intratracheally, intratumorally, intramuscularly, endoscopically, intralesionally, percutaneously, subcutaneously, regionally, or by direct injection or perfusion. In some aspects, administering the anti-cancer therapy and/or p38 MAPK inhibitor comprises local, regional or systemic administration. In other aspects, the anti-cancer therapy and p38 MAPK inhibitor are administered essentially concomitantly. In certain aspects, the anti-cancer therapy is administered before the p38 MAPK inhibitor. In some aspects, the anti-cancer therapy is administered after the p38 MAPK inhibitor. In certain aspects, the anti-cancer therapy and/or p38 MAPK inhibitor are administered two or more times.

In certain aspects, the method further comprises administering at least one other anti-cancer therapy. In some aspects, more than one p38 MAPK inhibitor is administered.

In some aspects, the cancer is resistant to a first anti-cancer therapy. In certain aspects, the first anti-cancer therapy is chemotherapy or radiotherapy.

In another embodiment, there is provided a pharmaceutical composition comprising a p38 MAPK inhibitor and anti-cancer therapy useful in treating a cancer patient who has been determined to have an elevated expression of FOXC2 relative to a reference level. In some aspects, the p38 MAPK inhibitor is SB 203580. In certain aspects, the anti-cancer therapy is Enzalutamide or Docetaxel.

In yet another embodiment, there is provided a method of predicting a response to a p38 MAPK inhibitor in combination with an anti-cancer therapy in a patient having a cancer comprising detecting the expression level of FOXC2 in the cancer cells of said patient, wherein if the cancer cells have an elevated expression of FOXC2 relative to a reference level, then the patient is predicted to have a favorable response to the p38 MAPK inhibitor in combination with an anti-cancer therapy. In certain aspects, a favorable response to a p38 MAPK inhibitor in combination with an anti-cancer therapy comprises reduction in tumor size or burden, blocking of tumor growth, reduction in tumor-associated pain, reduction in cancer associated pathology, reduction in cancer associated symptoms, cancer non-progression, increased disease free interval, increased time to progression, induction of remission, reduction of metastasis, or increased patient survival.

In another embodiment, there is provided a method of treating breast cancer metastasis in a subject comprising administering to said subject a p38 mitogen activated protein kinase (MAPK) inhibitor in an amount effective to treat. In some aspects, the subject is a human subject.

In certain aspects, treating comprises inhibiting the formation of metastases, inhibiting the growth of metastases, or killing circulating breast cancer cells. In some aspects, treating does not comprise inhibiting the growth of primary tumor cells. In some aspects, the circulating breast cancer cells are cancer stem cells. In particular aspects, the cancer stem cells are CD44^{high} and CD24^{low}.

In some aspects, the breast cancer is claudin-low breast cancer. In certain aspects, the breast cancer is triple negative breast cancer. In some aspects, the breast cancer metastasis is in the lungs.

In certain aspects, the method further comprises administering at least one other anti-cancer therapy. In some aspects, the anti-cancer therapy is chemotherapy, radiotherapy, gene therapy, surgery, hormonal therapy, anti-angiogenic therapy or cytokine therapy. In particular aspects, the hormonal therapy is an estrogen-receptor modulator. For example, the estrogen-receptor modulator is tamoxifen or letrozole.

In some aspects, the subject has previously received a radiotherapy, a chemotherapy, an immunotherapy, a molecularly targeted therapy or had surgical resection of a tumor.

In certain aspects, the subject has a decrease in FOXC2 expression relative to prior to administration of the p38 MAPK inhibitor. In some aspects, the subject has a decreased number of cancer stem cells relative to prior to administration of the p38 MAPK inhibitor. In certain aspects, the subject has decreased phosphorylation at serine 367 of FOXC2 relative to prior to administration of the p38 MAPK inhibitor.

In some aspects, the p38 MAPK inhibitor is SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745, DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804. In particular aspects, the p38 MAPK inhibitor is SB 203580. In some aspects, the p38 MAPK inhibitor is SB 203580 and the anti-cancer therapy is chemotherapy. In certain aspects, the p38 MAPK inhibitor is SB 203580 and the anti-cancer therapy is hormonal therapy.

In certain aspects, the anti-cancer therapy and/or p38 MAPK inhibitor are administered intravenously, intraperitoneally, intratracheally, intratumorally, intramuscularly, endoscopically, intralesionally, percutaneously, subcutaneously, regionally, or by direct injection or perfusion. In some aspects, administering the anti-cancer therapy and/or p38 MAPK inhibitor comprises local, regional or systemic administration. In certain aspects, the anti-cancer therapy and p38 MAPK inhibitor are administered essentially concomitantly. In some aspects, the anti-cancer therapy is administered before the p38 MAPK inhibitor. In certain aspects, the anti-cancer therapy is administered after the p38 MAPK inhibitor. In some aspects, the anti-cancer therapy and/or p38 MAPK inhibitor are administered two or more times. In some aspects, more than one p38 MAPK inhibitor is administered.

In some aspects, the breast cancer metastasis is resistant to a first anti-cancer therapy. In certain aspects, the first anti-cancer therapy is chemotherapy or radiotherapy.

In another embodiment, there is provided a pharmaceutical composition comprising a p38 MAPK inhibitor useful in treating a cancer patient with breast cancer metastasis. In some aspects, the p38 MAPK inhibitor is SB 203580. In certain aspects, the composition further comprises an anti-cancer therapeutic agent. In some aspects, the anti-cancer therapeutic agent is chemotherapy, gene therapy, hormonal therapy, anti-angiogenic therapy or cytokine therapy. In certain aspects, the anti-cancer therapeutic agent is chemotherapy. In certain aspects, the anti-cancer therapeutic agent is hormonal therapy.

A further embodiment provides a method of treating a BCR-ABL related disorder in a subject comprising administering to said subject a p38 mitogen activated protein kinase (MAPK) inhibitor, a glucocorticoid receptor agonist, and a tyrosine kinase inhibitor in an amount effective to treat the disorder. In particular aspects, said subject is a human subject.

In some aspects, treating comprises inhibiting the growth of primary tumor cells, inhibiting the formation of metastases, inhibiting the growth of metastases, killing circulating cancer cells, inhibiting the growth and/or survival of cancer stem cells, inducing remission, extending remission, or inhibiting recurrence. In particular aspects, treating comprises inhibiting mesenchymal stem cell-mediated TKI resistance.

In some aspects, the BCR-ABL related disorder is cancer. In certain aspects, the cancer is leukemia or lymphoma. In particular aspects, the leukemia is acute lymphoblastic leukemia (ALL), or chronic myeloid leukemia (CML).

In certain aspects, the TKI is selected from the group consisting of imatinib, dasatinib, nilotinib, bosutinib, ponatinib, bafetinib, saracatinib, tozasertib and rebastinib. In some aspects, the TKI is imatinib or dasatinib.

In some aspects, the glucocorticoid receptor agonist is dexamethasone, cortisol, cortisone, prednisolone, prednisone, methylprednisolone, trimcinolone, hydrocortisone, or corticosterone. In particular aspects, the glucocorticoid receptor is dexamethasone.

In certain aspects, the p38 MAPK inhibitor is SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745, DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804. In particular aspects, the p38 MAPK inhibitor is SB 203580. In specific aspects, the p38 MAPK inhibitor is SB 203580, the glucocorticoid receptor is dexamethasone, and the TKI is imatinib. In other aspects, the p38 MAPK inhibitor is SB 203580, the glucocorticoid receptor is dexamethasone, and the TKI is dasatinib.

In some aspects, the p38 MAPK inhibitor, glucocorticoid receptor agonist, and/or TKI are administered intravenously, intraperitoneally, intratracheally, intratumorally, intramuscularly, endoscopically, intralesionally, percutaneously, subcutaneously, regionally, or by direct injection or perfusion. In certain aspects, administering comprises local, regional or systemic administration. In some aspects, the glucocorticoid receptor, TKI, and p38 MAPK inhibitor are administered essentially concomitantly. In certain aspects, the glucocorticoid receptor and/or TKI is administered before the p38 MAPK inhibitor. In some aspects, the glucocorticoid receptor and/or TKI is administered after the p38 MAPK inhibitor. In particular aspects, the glucocorticoid receptor, TKI, and/or p38 MAPK inhibitor are administered two or more times. In some aspects, more than one p38 MAPK inhibitor is administered.

In certain aspects, the BCR-ABL related disorder is resistant to a first anti-cancer therapy. In some aspects, the first anti-cancer therapy is a TKI.

In some aspects, the method further comprises administering at least one other anti-cancer therapy. In certain aspects, the anti-cancer therapy is chemotherapy, radiotherapy, gene therapy, surgery, hormonal therapy, anti-angiogenic therapy or cytokine therapy.

Another embodiment provides a pharmaceutical composition comprising a p38 MAPK inhibitor, glucocorticoid receptor agonist, and TKI useful in treating a patient with a BCR-ABL related disorder. In some aspects, the p38 MAPK inhibitor is SB 203580.

In certain aspects, the glucocorticoid receptor agonist is dexamethasone. In some aspects, the TKI is imatinib or dasatinib. In some aspects, the BCR-ABL related disorder is ALL.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

Prostate Cancer

FIGS. 1A-1I: PSA^{-/lo} PCa stem-like cells, as well as androgen-independent PCa cell lines exhibit elevated FOXC2 expression and key properties defining the EMT/CSC phenotype. (A) The left panel shows FACS plots representing sorting of GFP⁺ (PSA⁺) and GFP^{-/lo} (PSA^{-/lo}) fractions from LNCaP cells. The right panels show morphology and GFP fluorescence of sorted cells. (B) qRTPCR analyses for FOXC2, and key prostate-epithelial-differentiation-(PD), neuroendocrine-differentiation- (NE), EMT- and stem-cell (SC)-related markers on sorted PSA⁺ and PSA^{-/lo} fractions from LNCaP cells analyzed immediately after sorting. Y-axis represents fold change in HPRT-normalized mRNA expression (n=3; error bars indicate SEM). (C) Immunoblotting for FOXC2 and other indicated markers on sorted PSA⁺ and PSA^{-lo} fractions. (D) IF for FOXC2 and indicated markers in sorted PSA⁺ and PSA^{-/lo} cells (scale bar=100 μm; DAPI). (E) qRTPCR analyses for FOXC2 and other indicated markers in PC3 and DU145 PCa cells compared to that in LNCaP cells. Y-axis represents fold-change in HPRT-normalized mRNA expression compared to that of LNCaP cells (n=3; error bars indicate SEM). (F) Immunoblotting for FOXC2 and other indicated markers in the above cells (***p<0.001). (G) Representative FACS plots for CD44 (APC) and CD24 (PE) surface marker expression analyzed in LNCaP and DU145 cells. (H) Quantification of FACS analysis shown in D (n=3; error bars indicate SEM). (I) Quantitation of prostospheres (n=5; error bars indicate SEM; ***p<0.001).

FIGS. 2A-2J: FOXC2 represents a critical convergence factor that is commonly up-regulated by multiple EMT-inducers in PCa cells, and its expression correlates with recurrent and high Gleason score prostate tumors associated with poor clinical prognosis. (A) Morphology of LNCaP cells after stable over-expression of EMT transcription factors-Zeb1 and Snail. (B) Morphology of DU145 cells after stable knockdown of Zeb1 and Snail (A, B: scale bar=100 μm). (C, E) qRTPCR analyses for indicated markers in 2A and 2B respectively. Y-axis represents fold-change in HPRT-normalized mRNA expression compared to that of Vector Control cells (n=3; error bars indicate SEM). (D, F) Immunoblotting for various markers in the indicated cell lines. (G) FOXC2 expression levels in recurrent vs non-recurrent clinical PCa data from the GDS4109 GEO database. (H). FOXC2 expression levels in prostate tumors of varying Gleason scores-data from GSE17356 (H), and TCGA (I) databases. (J) Quantitation of FOXC2 protein expression in various patient PCa tissues as analyzed by IHC (corresponding images shown in FIG. 8; BPH: Benign prostatic hyperplasia, PIN: Prostatic intraepithelial neoplasia, G7: Gleason 7).

FIGS. 3A-3O: FOXC2 is necessary and sufficient to confer EMT/CSC features, and the shift to androgen-independence/drug-resistance in PCa cells. (A) The upper panel shows morphology of LNCaP cells after FOXC2 over-expression; the lower panel shows reduction in PSA expression (in the same field) as inferred by reduced expression of GFP cloned downstream of the PSA promoter (scale bar=100 μm). (B) qRTPCR analyses for indicated markers. Y-axis represents fold change in HPRT-normalized mRNA expression compared to that of Vector Control cells (n=3; error bars indicate SEM). (C) Immunoblotting for various markers in the indicated cell lines. (D) Quantitation of prostospheres (n=5; error bars indicate SEM; **p<0.01). (E, F) Quantification of cell survival using MTS Assay in FOXC2 over-expressing LNCaP cells treated with Enzalutamide (E) or Docetaxel (F). Data are represented as absorbance (OD) at 490 nm (n=3), *p<0.05, **p<0.01. (G) Morphology of indicated cell types (scale bar=100 μm). (H) qRTPCR analyses for indicated markers in DU145 cells upon FOXC2 suppression. Y-axis represents fold change in HPRT-normalized mRNA expression compared to DU145-shFF3 cells (n=3; error bars indicate SEM). (I) Immunoblotting for various markers in the indicated cell types. (J) IF for AR/PSA expression in the indicated cell types (scale bar=100 μm). (K) Representative FACS plots for CD44 (APC) and CD24 (PE) surface marker expression analyzed in the indicated cell lines. (L) Graph shows quantification of FACS analysis shown above (n=3; error bars indicate SEM; ***p<0.001). (M) Quantitation of prostospheres formed per 1000 DU145 cells upon FOXC2 suppression (n=5; error bars indicate SEM; **p<0.01). (N, O) Quantification of cell survival using MTS Assay in DU145-shFOXC2 cells treated with Enzalutamide (N) or Docetaxel (O). Data are represented as absorbance (OD) at 490 nm (n=3), *p<0.05.

FIGS. 4A-4F: FOXC2 regulates AR expression and stem-cell properties in PCa cells via Zeb1. (A) Immunoblotting in LNCaP cells expressing indicated constructs. (B) Quantitation of the number of tumorspheres formed per 1000 LNCaP cells, each expressing indicated constructs (n=5; error bars indicate SEM; *p<0.05, ***p<0.001). (C) Quantification of cell survival using MTS Assay in various LNCaP-derived lines treated with Enzalutamide. Data are represented as absorbance (OD) at 490 nm (n=3), **p<0.01. (D) Immunoblotting for Zeb1, FOXC2, AR and Actin in DU145 cells expressing indicated constructs. (E) Quantitation of prostospheres formed per 1000 DU145 cells each expressing indicated constructs (n=5; error bars indicate SEM; ***p<0.001). (F) Quantification of cell survival using MTS Assay in various DU145-derived cell lines treated with Enzalutamide. Data are represented as absorbance (OD) at 490 nm (n=3), *p<0.05, ***p<0.001.

FIGS. 5A-5I: Activation of p38MAPK signaling consistently correlates with the FOXC2-dependent EMT/CSC state in androgen-independent PCa cells. Immunoblotting for total (t)- and phospho (p)-p38 and its target substrate ATF2 in PSA⁺, PSA^{-/lo} cells (A), PCa cell lines (B), LNCaP cells after over-expression of FOXC2 (C), and DU145 cells after suppression of FOXC2 expression (D). (E) Immunoblotting for FOXC2, p- and t-Smad2/3 and p38 signaling components in LNCaP cells induced to undergo EMT using TGFβ1 (a physiological activator of p38 signaling), as well as in DU145 cells treated with LY364947, an inhibitor of TGFβ1 signaling. F. Quantitation of tumorspheres (n=5; error bars indicate SEM; *p<0.05, ***p<0.001). (G) Schematic shows the putative p38MAPK phosphorylation site on human FOXC2 protein based on phospho-motif scan. (H) Quantitation of tumorspheres (n=5; error bars indicate SEM; **p<0.01, ***p<0.001). (I) Immunoblotting in LNCaP cells expressing the indicated constructs.

FIGS. 6A-6K: Suppression of p38 signaling in androgen-independent cells results in reversal of EMT, significant decrease in FOXC2-dependent stem-like properties, and restoration of sensitivity to Enzalutamide and Docetaxel. (A) Morphology of DU145 cells upon SB203580 (specific p38 signaling inhibitor) treatment for 7 days. (B) Representative images of wound healing assay performed with DU145 cells treated with SB203580. (C) Quantitation of cell migration in B (n=5; ***p<0.001; error bars indicate SEM). (D) qRTPCR analyses for various markers in SB203580-treated DU145 cells. Y-axis represents fold-change in HPRT-normalized mRNA expression in SB203580-treated cells compared to vehicle-treated cells, relative to day 0 (n=3; error bars indicate SEM). (E) Immunoblotting for FOXC2, key EMT-markers and p38 signaling components in DU145 cells upon SB203580-treatment for 7 days. F. Representative FACS plots for CD44 (APC) and CD24 (PE) surface marker expression in DU145 cells treated with vehicle or SB203580 for 7 days. (G) Quantification of FACS analysis shown in panel F (n=3; error bars indicate SEM; ***p<0.001). (H) Quantitation of tumorspheres formed per 1000 DU145 cells (treated either with vehicle or SB203580 for 7 days; n=5, error bars indicate SEM, ***p<0.001). (I) IF staining for AR and PSA in DU145 cells treated with vehicle or SB203580 for 7 days (scale bar=100 m). (J, K) Quantification of cell survival using MTS Assay in DU145 cells treated as indicated, with Enzalutamide (J) or Docetaxel (K). Data are represented as absorbance (OD) at 490 nm (n=3), *p<0.05, **p<0.01, ***p<0.001.

FIGS. 7A-7I: Combinatorial treatment of mice bearing aggressive androgen-insensitive tumors with both SB203580 and Enzalutamide, results in significant regression of primary tumor formation as well as marked loss in circulating tumor cell population. (A) Schematic shows the design for in vivo experiments. (B) Quantification of luminescence of luciferase activity in tumors formed by DU145-RFP-Luciferase-labeled cells, and treated as indicated. (C) Tumors isolated from mice treated as indicated (scale bar=1 cm, n=8 data-points). (D, E) Graphs show tumor volume (D) and tumor weight (E) respectively in indicated conditions (ns=p>0.05, **p<0.01). (F) qRTPCR analyses for FOXC2, and other indicated markers in isolated tumors. Y-axis represents fold-change in HPRT-normalized mRNA expression in randomly selected tumor samples compared to that in pooled control vehicle-treated tumors (n=3; error bars indicate SEM). (G) IHC for FOXC2, AR and pATF2 (a marker for activated p38 signaling) on indicated tumors. (H) Quantification of colonies formed by CTCs isolated from blood of mice bearing various tumors as indicated. The colonies were confirmed to be of human origin by RFP expression that was stably introduced into DU145 cells (ns=p>0.05, ***p<0.001). (I) Schematic depicting the participation of FOXC2 in the reprogramming of differentiated/epithelial prostate cancer cells into ADT-insensitive, drug-resistant/neuroendocrine stem-like cells lacking epithelial traits.

FIG. 8: Immunohistochemical analysis of FOXC2 expression in primary human prostate tissue samples representing BPH (Benign prostatic hyperplasia), PIN (Prostatic intraepithelial neoplasia), and Gleason Grade 7. Shown are 3 distinct samples representing each condition. BC: Human Breast Cancer tissue sample, positive control (scale bar=100 μm).

FIG. 9: Immunohistochemical analyses demonstrating that primary human prostate small cell carcinoma tissue characterized by lack of AR expression, exhibits high level of FOXC2 expression (scale bar=50 μm). Insert in the right panel shows a higher magnification of the FOXC2-stained tissue.

FIG. 10: Immunoblot analyses demonstrating reciprocal relationship between expression of FOXC2 and AR in lysates of various human patient-derived tumor xenograft (PDX) sublines that model lethal variant small cell prostate carcinoma with AR-negative neuroendocrine features [144-13 and 177-0: AR-negative sublines; 133-4 and 180-30: AR-positive controls].

FIG. 11: qRT-PCR analyses for prostate differentiation markers—AR and PSA—in DU145 cells, performed progressively from days 0-7 after SB203580 treatment. Y-axis represents fold change in HPRT-normalized mRNA expression in SB203580-treated samples compared to that in control cells (n=3; error bars indicate SEM).

FIG. 12: RFP-luciferase-labeled DU145 prostate tumor cells were subcutaneously injected into NOD/SCID mice and tumor progression monitored by bioluminescent imaging. After formation of palpable tumors (approximately 2 weeks), mice were randomly divided into 4 groups, and treated as indicated. Panel shows mice and luminescence of luciferase activity discernable at the end of the treatment schedule (n=8 data points).

Breast Cancer

FIGS. 13A-13F: FOXC2 expression correlates with p38 activation in cells with mesenchymal and stem cell properties. (A) Alignment of FOXC2 amino acid sequences from multiple species shows high evolutionary sequence conservation at S367, the putative phosphorylation site for p38. (B) Cell lysates from the indicated cells were analyzed by immunoblotting for p-p38, p38 and FOXC2. β-actin was used as a loading control. (C) The indicated cells were treated with vehicle or SB203580 for 24 h. Cell lysates were analyzed by immunoblotting for FOXC2. β-actin was used as a loading control (D) The indicated cells were transduced with p38 shRNA (shp38) or control shRNA (shControl). Cell lysates were analyzed by immunoblotting for p38 and FOXC2. β-actin was used as a loading control. (E) Pre-treatment of the indicated cells with 10 μM MG132 prevents the proteolytic degradation of FOXC2 following SB203580 treatment, as determined by immunoblotting. β-actin was used as a loading control. (F) For the wound healing assay, a confluent monolayer culture of epithelial HMLE cells was scratched with a sterile pipette tip. HMLE cells were treated with vehicle or SB203580 and fixed immediately following scratch induction (0 h) or 9 h post-wound induction, followed by immunostaining for FOXC2 and p-p38. Nuclei were counterstained with DAPI. Scale bar, 20 μm.

FIGS. 14A-14E: p38 inhibition leaves primary tumor growth unabated but significantly compromises metastasis. (A) 4T1 cells were treated with vehicle or SB203580 for 24 h. Cell lysates were analyzed by immunoblotting for Foxc2, with β-actin as a loading control. (B) Luciferase-labeled 4T1 cells were orthotopically injected into mice, subsequently treated daily with vehicle or SB203580. Primary mammary tumors (left panel; macroscopic) and lungs (right panel; bioluminescence) were harvested at 3, 4, 5 and 6 weeks post-implantation. n=5 mice per group. (C) The size of the primary mammary tumors, harvested from mice in (b), was measured with a caliper as the product of two perpendicular diameters (mm²) and plotted over time. (D) The bioluminescent signal from the lungs in (B) was quantified to determine the incidence of metastases. (E) The number of CTCs per 100 μl of blood, isolated from mice in (B), was quantified and plotted over time. p-values were calculated using Student's unpaired two-tailed t-test. *p<0.05; ***p<0.001 compared to the control.

FIGS. 15A-15L: p38 inhibition compromises the acquisition and maintenance of EMT and stem cell properties in vitro. (A) MCF10A cells treated with TGFβ1 alone, or in combination with SB203580, for 3 days. Cells were harvested and the corresponding lysates were analyzed by immunoblotting for FOXC2, E-cadherin and mesenchymal markers. β-actin was used as a loading control. (B) HMLE-Snail-ER cells were treated with 4-OHT for 12 days and concurrently exposed to vehicle or SB203580. Cells were harvested at the indicated timepoints and the corresponding lysates were analyzed by immunoblotting for FOXC2, E-cadherin and mesenchymal markers. β-actin was used as a loading control. (C) HMLE-Snail-ER and HMLE-Twist-ER cells were treated with 4-OHT for 12 days and concurrently exposed to vehicle or SB203580. One thousand cells were seeded per well in ultra-low attachment plates and cultured for 7-10 days. Spheres with a diameter greater than 75 μm were counted. The data are reported as the number of spheres formed/1000 seeded cells±SEM. (D) The percentage of CD44^{high}/CD24^{low} cells in 4-OHT-treated HMLE-Snail-ER and HMLE-Twist-ER populations, concurrently exposed to vehicle or SB203580, was determined by FACS. Data are presented as mean±SEM. (E) HMLE-Twist-ER cells, transduced with p38 shRNA (shp38) or control shRNA (shControl), were treated with 4-OHT for 9 days and harvested at the indicated timepoints. The corresponding lysates were analyzed by immunoblotting for FOXC2, E-cadherin and mesenchymal markers. β-actin was used as a loading control. (F) The sphere-forming efficiency of 4-OHT-treated HMLE-Twist-ER cells, transduced with control shRNA (shControl) or p38 shRNA (shp38), was determined. The data are reported as the number of spheres formed/1000 seeded cells±SEM. (G) The indicated cells were treated with vehicle or SB203580, and the corresponding lysates analyzed by immunoblotting for FOXC2, E-cadherin and mesenchymal markers. β-actin was used as a loading control. (H) The sphere-forming efficiency of the indicated cells was determined in the presence of vehicle or SB203580. The data are reported as the number of spheres formed/1000 seeded cells±SEM. (I) The percentage of CD44^{high}/CD24^{low} subpopulations in indicated cells, treated with vehicle or SB203580, was determined by FACS. Data are presented as mean±SEM. (J) The relative wound closure by the indicated cells, treated with vehicle or SB203580, was measured by image analysis and represented in a graphical format. Data are presented as mean±SEM. (K) The relative wound closure by HMLE cells, transduced with control shRNA (shControl), p38 shRNA (shp38) or FOXC2 shRNA (shFOXC2), was measured by image analysis and represented in a graphical format. Data are presented as mean±SEM. (l) HMLE-Snail and HMLE-Twist cells were plated on FITC-conjugated gelatin and treated with vehicle or SB203580. After 16 h, the cells were fixed and stained with fluorescent phalloidin, which binds to F-actin, and the nuclei were counterstained with DAPI to facilitate visualization of the cells (see FIG. 9). Degradation of FITC-gelatin was quantified by image analysis. n=150 cells/sample. Data are reported as mean±SEM. p-values were calculated using Student's unpaired two-tailed t-test. *p<0.05; **p<0.01; ***p<0.001 compared to the control.

FIGS. 16A-16F: EMT and stem cell properties of HMLER cells expressing FOXC2 (S367) mutants. (A) HMLER cells were transduced with empty vector, FOXC2, FOXC2 (S367E), or FOXC2(S367A), and their morphology was imaged through phase-contrast microscopy. Scale bar, 100 m. (B) Cell lysates from HMLER cells, transduced with the indicated constructs, were analyzed by immunoblotting for FOXC2 (anti-HA), E-cadherin, fibronectin, and vimentin. β-actin was used as a loading control. (C) Sphere formation by the indicated cells is represented as the mean number of spheres formed/1000 seeded cells±SEM. (D) The indicated cells were analyzed by FACS for the presence of CD44 and CD24 on the cell surface. The circles denote the position of the vector-transduced control population in the cytograms. (E) Sphere formation by the indicated cells, treated with vehicle or SB203580, is represented as the mean number of spheres formed/1000 seeded cells±SEM. (F) The relative wound closure by the indicated cells, treated with vehicle or SB203580, was measured by image analysis and represented in a graphical format. Data are presented as mean±SEM. p-values were calculated using Student's unpaired two-tailed t-test. *p<0.05; **p<0.01; ***p<0.001 compared to the control.

FIGS. 17A-17E: p38-mediated phosphorylation of FOXC2 at S367 regulates metastasis. (A) 4T1 cells, transduced with either empty vector (pMIG) or FOXC2 (S367E), were treated with vehicle or SB203580. Cell lysates were analyzed by immunoblotting for FOXC2, with β-actin as a loading control. (B) 4T1 cells, transduced with empty vector or FOXC2 (S367E), were subjected to a sphere-formation assay in the presence of vehicle or SB203580. Data are presented as the mean number of spheres formed/1000 seeded cells±SEM. (C) 4T1-vector or 4T1-FOXC2 (S367E) luciferase-labeled cells were orthotopically injected into mice. Mice were treated daily with vehicle or SB203580, and primary tumor growth was monitored weekly by bioluminescence. Data are presented as the total photon flux plotted over time and reported as mean±SEM. (D) Representative bioluminescent images of lungs harvested from mice in (c) at 4 weeks post-implantation. (E) The bioluminescent signal from the lungs harvested from mice in (c) was quantified to determine the incidence of metastasis. Data are reported as mean±SEM. p-values were calculated using Student's unpaired two-tailed t-test.*p<0.05; ***p<0.001 compared to the control.

FIGS. 18A-18L: p38-mediated phosphorylation of FOXC2 directly regulates ZEB1 expression. (A) HMLER cells were transduced with empty vector (HMLER-vector) or FOXC2 (HMLER-FOXC2) and the transcript levels of FOXC2 and ZEB1 were determined by qRT-PCR, with glyceraldehyde 3-phosphate dehydrogenase (GAPDH) as the reference gene to normalize the variability in template loading. Data are reported as mean±SEM. (B) Cell lysates from the indicated cells were analyzed by immunoblotting for FOXC2 and ZEB1. β-actin was used as a loading control. (C) HMLE-Snail and HMLE-Twist cells were immunostained with antibodies against FOXC2 and ZEB1. Nuclei were counterstained with DAPI. Scale bar, 20 μm. (D) HMLE-Snail cells, transduced with control shRNA (shControl) or FOXC2 shRNA (shFOXC2), were immunostained with antibodies against FOXC2 and ZEB1. Nuclei were counterstained with DAPI. Scale bar, 20 m. (E) The relative expression of ZEB1 mRNA in the indicated cells, transduced with control shRNA (shControl) or FOXC2 shRNA (shFOXC2), was determined by qRT-PCR with GAPDH as the reference gene. Data are reported as mean±SEM. (F) The protein levels of FOXC2, ZEB1 and β-actin in the indicated cells, transduced with control shRNA (shControl) or FOXC2 shRNA (shFOXC2), were analyzed by immunoblotting. (G) The relative levels of miR200b and miR200c in HMLER-vector and HMLER-FOXC2 cells were determined by qRT-PCR, with U6 small nuclear RNA as an internal control. Data are reported as mean±SEM. (H) The mRNA abundance of miR200b and miR200c in HMLE-Snail cells, transduced with control shRNA (shControl) or FOXC2 shRNA (shFOXC2), was determined by qRT-PCR, with U6 small nuclear RNA as an internal control. Data are reported as mean±SEM. (I) A chromatin immunoprecipitation assay was performed using HMLER-FOXC2 cells to show that FOXC2 binds upstream to the transcriptional start site of the ZEB1 promoter. The y axis represents the percentage of bound FOXC2 and the x axis denotes the distance from the ZEB1 transcription start site in kb. (J) The relative levels of ZEB1 transcripts in the indicated cells, treated with vehicle or SB203580, were determined by qRT-PCR, with GAPDH as the reference gene. Data are reported as mean±SEM. (K) Cell lysates from the indicated cells, treated with vehicle or SB203580, were analyzed by immunoblotting for ZEB1. β-actin was used as a loading control. (L) HMLER cells were transduced with empty vector, FOXC2, FOXC2(S367E), or FOXC2(S367A). These cells were treated with vehicle or SB203580 and the corresponding lysates were analyzed by immunoblotting for ZEB1. β-actin was used as a loading control. p-values were calculated using Student's unpaired two-tailed t-test. *p<0.05; **p<0.01; ***p<0.001 compared to the control.

FIGS. 19A-19D: SB203580 treatment decreases FOXC2 immunostaining but neither SB203580 nor p38 shRNA impact FOXC2 transcript levels. (A) The indicated cells were immunostained with antibodies against p-p38 and FOXC2. Nuclei were counterstained with DAPI. Scale bar, 20 μm. (B) The indicated cells were treated with vehicle or SB203580 and subsequently immunostained with antibodies against FOXC2. Nuclei were counterstained with DAPI. Scale bar, 20 μm. (C) The relative expression of FOXC2 mRNA in the indicated cells, treated with vehicle or SB203580, was determined by qRT-PCR. GAPDH was used as the reference gene. (D) The relative expression of FOXC2 mRNA in the indicated cells, transduced with control shRNA (shControl) or p38 shRNA (shp38), was determined by qRT-PCR. GAPDH was used as the reference gene.

FIGS. 20A-20C: p38 interacts with FOXC2 and phosphorylates it at S367. (A, B) HEK293T cells were transfected with Myc-FOXC2 and HA-p38 or a kinase-dead mutant of p38 (HA-p38-DN) and subjected to immunoprecipitation (IP) with anti-HA (p38), anti-Myc (FOXC2) or control IgG followed by immunoblotting (IB) with antibodies as indicated. (C) Recombinant GST-FOXC2 fusion proteins: N-terminally truncated FOXC2 (amino acids 245-501), C-terminally truncated FOXC2 (amino acids 1-244), or N-terminally truncated FOXC2 (amino acids 245-501) with alanine substitution at serine 367 (S367A), were purified from E. coli using glutathione-sepharose-4B beads. The respective eluates were subjected to in vitro kinase assays with recombinant active p38. The reaction mixtures were resolved by SDS-PAGE and the phosphorylated proteins visualized by autoradiography. The electrophoretic mobility of phosphorylated GSTFOXC2 is indicated with an arrowhead. The GST control and the C-terminally truncated FOXC2 (amino acids 1-244), devoid of the putative p38 phosphorylation site, did not show any phosphorylation in this assay. The bottom panel depicts Coomassie blue staining of the protein input.

FIGS. 21A-21D: Monitoring mammary tumor progression and the effect of SB203580 treatment. (A) Luciferase-labeled 4T1 cells were orthotopically injected into mice, subsequently treated daily with vehicle (left panels) or SB203580 (right panels). Bioluminescent imaging was used to monitor weekly primary tumor growth. (B) The bioluminescent signal from the primary tumors from mice in (a) was quantified and plotted as the total photon flux emitted by the primary mammary tumors over time. (C) Macroscopic images of the lungs from mice in (a), harvested at 3, 4, 5 and 6 weeks, after implantation and treatment. n=5 mice per group. Veh=vehicle; SB=SB203580. (D) Hematoxylin and eosin staining of lung sections, harvested from mice described in (a), at 5 weeks after implantation and treatment. Scale bar, 100 μm.

FIGS. 22A-22D: p38 inhibition compromises colonization in an experimental metastasis model. (A) Luciferase-labeled MDA-MB-231 cells were injected into NOD/SCID mice via the tail vein. Starting 48 h post-implantation, the mice were treated daily with vehicle or SB203580 (n=6 mice per group). The emergence of lung metastases was monitored by bioluminescent imaging. Representative bioluminescent images, at 8 weeks post-implantation, are shown. (B) The Kaplan-Meier event-free survival curves of mice, injected with luciferase-labeled MDA-MB-231 cells via the tail vein, and subsequently treated daily with vehicle or SB203580, were generated. n=6 mice per group. The Gehan-Breslow-Wilcoxon method was used to compare the Kaplan-Meier survival curves and compute the corresponding p values. (C) Luciferase-labeled MDA-MB-231 cells, transduced with either control shRNA (shControl) or p38 shRNA (shp38), were injected into NOD/SCID mice via the tail vein (n=7 mice per group). The emergence of lung metastases was monitored by bioluminescent imaging. Representative bioluminescent images, at 9 weeks post-implantation, are shown. (D) The Kaplan-Meier event-free survival curves of mice injected via the tail vein with luciferase-labeled MDA-MB-231 cells, transduced with p38 shRNA (shp38) or control shRNA (shControl), were generated. n=7 mice per group. The Gehan-Breslow-Wilcoxon method was used to compare the Kaplan-Meier survival curves and compute the corresponding p values.

FIG. 23: p38 inhibition compromises the formation of invadopodia. Snail cells were plated on FITC-conjugated gelatin and treated with vehicle or SB203580. After 16 h, the cells were fixed and stained with fluorescent phalloidin, which binds to F-actin, and the nuclei were counterstained with DAPI to facilitate visualization of the cells. Areas of gelatin degradation, appearing as punctate black areas beneath the cells, are indicated by white arrows. Representative images are shown. Scale bar, 20 μm.

FIG. 24: ZEB1 is one of the most highly upregulated genes in HMLER-FOXC2 cells, relative to vector-transduced counterparts, as determined by microarray analysis. Microarray analyses of the gene expression profiles of HMLER cells overexpressing FOXC2 (HMLER-FOXC2) relative to vector-transduced counterparts (HMLER-vector). The platform used was the Affymetrix Human Genome U133 Plus 2.0 Array, and the data were deposited in the Gene Expression Omnibus under the GEO accession number GSE44335. The analysis, using a 5-fold change cut-off and a statistical significance false discovery rate (FDR)<0.05, yielded a total of 740 genes. The heatmap represents the differential expression of genes in epithelial HMLER-vector cells and mesenchymal HMLER-FOXC2 cells. Each row of the heatmap represents a specific gene probe. Each column of the heatmap represents a sample from HMLER-vector (Vector_1, Vector_2, Vector_3) or HMLER-FOXC2 (FOXC2_1, FOXC2_2, FOXC2_3) cells, as indicated. Each colored cell in the heatmap represents the gene expression value for a specific probe in the respective sample. The positions of the cells corresponding to different ZEB1 and FOXC2 probes are indicated with arrows. The corresponding fold-changes in gene expression, in HMLER-FOXC2 compared to HMLER-vector cells, are shown to the right of the heatmap.

FIGS. 25A-25D: p38 inhibition compromises stem cell properties in vitro. (A) HMLER-Snail cells were treated with various p38 inhibitors. Cells were seeded in ultra-low attachment plates and cultured for 7-10 days. Spheres with a diameter greater than 75 m were counted. The data are reported as the number of spheres formed/1000 seeded cells±SEM. Concentration of the drug that induced 50% reduction in sphere formation: SB203580—20 uM; PH797804—10 nM; LY2228820—10 nM; VX-702—50 uM. (B) SUM159 cells were treated with various p38 inhibitors. Cells were seeded in ultra-low attachment plates and cultured for 7-10 days. Spheres with a diameter greater than 75 μm were counted. The data are reported as the number of spheres formed/1000 seeded cells±SEM. Concentration of the drug that induced 50% reduction in sphere formation: SB203580—20 uM; PH797804—100 nM; LY2228820—300 nM nM; VX-702-50 uM. (C) HMLE-Snail cells were treated with various p38 inhibitors. Cells were seeded in ultra-low attachment plates and cultured for 7-10 days. Spheres with a diameter greater than 75 μm were counted. The data are reported as the number of spheres formed/1000 seeded cells±SEM. Concentration of the drug that induced 50% reduction in sphere formation: SB203580—20 uM; PH797804—300 nM; LY2228820—3 μM; VX-702 did not inhibit sphere formation. (D) Western blots of HMLER-Snail cell treatment with various p38 inhibitors on Day 2 of treatment. Concentration of the drug that induced 50% reduction in FOXC2 protein: SB203580-10 uM; PH797804—50 nM; TAK-714-5 uM.

Leukemia

FIGS. 26A-26D: Screening a library of clinical compounds identified those that prevent mesenchymal stem cell (MSC)-mediated support to BCR-ABL-positive (BCR-ABL+) acute lymphoblastic leukemia (ALL) cells. (A) Schematic outline for screening the library of clinical compounds. MSCs were pretreated with imatinib (IM; 5 μM) and an individual clinical compound (6.6 μM for all compounds, except for dexamethasone [DEX, 50 nM]) for 3 days before luciferase-labeled BCR-ABL+ mouse ALL cells were seeded. Treatment was continued for 1 day, and co-cultured cells were examined for leukemic cell clusters through phase-contrast microscopy and for toxicity through bioluminescence imaging. (B) Categorization of 146 clinical compounds into four groups on the basis of their toxicity to MSCs or leukemic cells and their effect on leukemic cell cluster formation. Note that group 4 compounds (n=99) failed to prevent the cluster formation and even some compounds from group 4 increased the number of clusters (++). NA, not applicable. (C) Compounds from groups 1 and 2 prevented imatinib (IM)-induced leukemic cell cluster formation underneath the MSCs. Quantification of leukemic cell clusters formed underneath MSCs pretreated with a group 1 (3-10) or group 2 (11-15) compound. Data are shown as the means±standard error of the mean. (D) Key signaling pathway members targeted by group 1 and 2 compounds.

FIGS. 27A-27F: Combining SB203580 (SB) or dexamethasone (DEX) treatment with imatinib (IM) eliminates mesenchymal stem cell (MSC)-mediated support to BCR-ABL-positive (BCR-ABL+) acute lymphoblastic leukemia (ALL) cells. (A) SB203580 prevented leukemic cell cluster formation beneath the imatinib-pretreated MSCs. MSCs were pretreated (Pre) under the indicated conditions for 4 days before luciferase-expressing leukemic cells were seeded, and treatment was continued for 24 hours. Left, microscopic images of the co-cultured leukemic cells and MSCs (dark, small, round cells are clustered leukemic cells beneath MSCs, and bright, small, round cells are leukemic cells suspended in medium). Right, quantification of ALL cell clusters. (B) Bioluminescence imaging of co-cultured leukemic cells/MSCs shown in panel A (luminescent intensity indicates total number of live leukemic cells in the culture). (C) SB203580 co-pretreatment with imatinib induces apoptosis of leukemic cells. Leukemic cells from the co-cultured samples shown in panel A were stained with Annexin V and analyzed by flow cytometry. Note that in the presence of imatinib (5 μM), addition of SB203580 (20 μM) to the culture prevented leukemic cell cluster formation and sensitized leukemic cells to imatinib treatment. SSC, side scatter. (D) Dexamethasone targets leukemic cell clusters formed underneath imatinib-pretreated MSCs. MSCs were pretreated under the indicated conditions for 4 days before luciferase-expressing leukemic cells were seeded, and treatment was continued for 24 hours. Left, microscopic images of co-cultured leukemic cells/MSCs. Right, quantification of ALL cell clusters. (E) Bioluminescence imaging of co-cultured leukemic cells/MSCs shown in panel D (luminescent intensity indicates total number of live leukemic cells in the culture). (F) Dexamethasone co-pretreatment with imatinib induces apoptosis of leukemic cells. Leukemic cells from the co-cultured samples described in panel D were stained with Annexin V and analyzed by flow cytometry. Note that in the presence of imatinib (5 μM), dexamethasone (50 nM) effectively targeted clustered leukemic cells. Scale bars, 50 m. Data are shown as means±standard error of the mean. *P<0.05, as determined by t-test.

FIGS. 28A-28B: SB203580 (SB) co-treatment with imatinib (IM) reverses imatinib-induced molecular alterations in mesenchymal stem cells (MSCs). (A) Addition of SB203580 to imatinib treatment prevented activation of p38 MAPK in MSCs. MSCs were starved in serum-free medium for 2 hours and treated with imatinib and/or SB203580 for 30 minutes. MSCs were supplemented with 10% serum, and the treatment was continued for 30 minutes. Protein lysates from MSCs treated under the indicated conditions were subjected to immunoblot analysis. α-tubulin was used as a loading control. (B) SB203580+imatinib prevents imatinib-induced gene expression alterations in MSCs. MSCs were treated under the indicated conditions for 2 days, and real-time reverse transcription polymerase chain reaction analysis was performed on selected genes known to be induced (Sort1, Adipoq, Rspo2, Cxcl12, Fgf10, Fgfr2, IL-7, Sphk1, and Ogn) or suppressed (Timp3) with imatinib treatment in MSCs. Data are shown as means±standard error of the mean.

FIGS. 29A-29D: Combining SB203580 (SB) with dasatinib and dexamethasone (DEX) prevents the development of tyrosine kinase inhibitor (TKI) resistance in BCR-ABL-positive (BCR-ABL+) acute lymphoblastic leukemia (ALL). (A) Progression of leukemia in mice treated under the indicated conditions. Non-obese diabetic severe combined immunodeficient mice were transplanted intravenously with luciferase-labeled leukemic cells (2×10⁶ cells), and engraftment of injected leukemic cells was confirmed by bioluminescence imaging. Five days after transplantation, treatment was initiated. Mice were divided into the following treatment groups: vehicle (n=3), SB203580 (n=3), dasatinib (n=3), dasatinib+dexamethasone (n=8), and dasatinib+dexamethasone+SB203580 (n=8). Leukemia burden was monitored by bioluminescence imaging at regular intervals. Luminescence signals were adjusted to the same scale at each time point for all treatment groups. (B) Kaplan-Meier survival analysis of mice in panel A. Arrow indicates the beginning of treatment (5 days after transplantation). P values were determined by the log-rank test. (C) Apoptosis analysis of mCherry-positive (mCherry+) leukemic cells in the bone marrow samples of leukemic mice. Upper panel, bioluminescence images of mice treated under indicated conditions (n=2 per group). Lower panel, analysis of apoptosis by flow cytometry of mCherry+ leukemic cells. Representative data shown in flow cytometry plots in the lower panel correspond to the groups of mice shown in the upper panel. (D) Examination of mCherry+ leukemic cells in the peripheral blood at day 23 and day 27 after transplantation.

FIG. 30: Working model of imatinib-induced mesenchymal stem cell (MSC)-mediated drug resistance and an effective strategy to overcome BCR-ABL-positive (BCR-ABL+) acute lymphoblastic leukemia (ALL) resistance to tyrosine kinase inhibitors (TKIs). In the absence of imatinib (first from left), the growth of BCR-ABL+ALL cells is driven by BCR-ABL signaling. In the presence of imatinib (IM; second from left), MSCs undergo morphological and functional changes and produce multiple supportive molecules, thus activating alternative signaling pathways in ALL cells while BCR-ABL signaling is blocked by imatinib. As a result, the BCR-ABL+ ALL cells switch from BCR-ABL signaling to alternative signaling for survival. Imatinib+SB203580 (SB; third from left) reverses imatinib-mediated morphological and functional changes in MSCs and prevents MSC-mediated alternative survival support to leukemic cells. Imatinib+dexamethasone (DEX; fourth from left) also effectively prevents MSCs from providing alternative survival support by indirectly targeting survival signaling in leukemic cells. Therefore, combining a TKI with a p38 MAPK inhibitor and dexamethasone (fifth from left) could help prevent development of TKI resistance in BCR-ABL+ ALL. The elongated or polygonal cells are MSCs whereas the small round cells are leukemic cells (dark cells are live cells and grey cells are apoptotic cells).

FIGS. 31A-31D: Screening library of clinical compounds identified those that prevent leukemic cell clusters from forming underneath imatinib (IM)-pretreated mesenchymal stem cells (MSCs). (A, B, and C) Effect of combination of individual clinical compounds with imatinib on imatinib-induced leukemic cell clusters. Top, layout for the distribution of clinical compounds in 96-well plate 1 (A), 2 (B), or 3 (C). Bottom, number of leukemic cell clusters (average number of clusters from 3 different fields) observed after co-treatment with imatinib and the clinical compound from plate 1 (A), 2 (B), or 3 (C). Note that compounds in the highlighted wells effectively prevented leukemic cell clusters from forming underneath imatinib-pretreated MSCs. NA indicates compounds that showed toxicity to leukemic cells and/or MSCs; hence, these wells did not yield leukemic clusters. (D) Effect of combination of individual clinical compounds with imatinib on leukemic cell viability. MSCs were pretreated with imatinib (5 μM) and an individual clinical compound (6.6 μM for all compounds, except for dexamethasone [DEX, 50 nM]) for 3 days before luciferase-labeled BCR-ABL-positive (BCR-ABL+) mouse acute lymphoblastic leukemia (ALL) cells were seeded. Treatment was continued for 1 day, and co-cultured cells were assayed for toxicity via bioluminescence imaging. Each row of the compounds in plates 1, 2, and 3 was used to treat co-cultured MSCs/leukemic cells in a 12-well plate. Note that empty wells were not subjected to any treatment.

FIGS. 32A-32B: SB203580 (SB) co-treatment with imatinib (IM) does not affect leukemic cell proliferation or apoptosis in the absence of mesenchymal stem cells (MSCs). (A) Proliferation assay of BCR-ABL-positive (BCR-ABL+) acute lymphoblastic leukemia (ALL) cells treated with SB203580 (20 μM) alone or in combination with imatinib (10 nM, 50 nM, 0.1 μM, 0.5 μM, or 1 μM for 2 days. Leukemic cells were cultured in the absence of MSCs and IL-7. Data are shown as means±standard error of the mean. (B) Effect of SB203580 co-treatment with imatinib on leukemic cell apoptosis. Data were obtained after 2 days of culture without MSCs and IL-7 under the indicated conditions. Leukemic cells were stained with Annexin V and analyzed by flow cytometry.

FIG. 33: SB203580 (SB) prevents leukemic cell cluster formation underneath imatinib (IM)-pretreated mesenchymal stem cells (MSCs). MSCs were pretreated (Pre) under the indicated conditions for 4 days before luciferase-expressing leukemic cells were seeded; then, treatment was continued for 24 hours. Broader microscopic views corresponding to the images from FIG. 27A are shown. IM-, vehicle control. Scale bars, 50 μm.

FIG. 34: SB203580 (SB) prevents human leukemic cell cluster formation underneath imatinib (IM)-pretreated mesenchymal stem cells (MSCs). MSCs were pretreated (Pre) under the indicated conditions for 4 days before BCR-ABL-positive (BCR-ABL+) human B-cell acute lymphoblastic leukemia (ALL) cells were seeded, and treatment was continued for 24 hours. Left, Microscopic images of the co-cultured leukemic cells/MSCs. Right, quantification of ALL cell clusters. Scale bars, 50 μm. Data are shown as means±standard error of the mean. *P<0.05, as determined by t-test.

FIG. 35: SB203580 (SB) pretreatment of leukemic cells does not prevent formation of leukemic cell clusters underneath imatinib (IM)-pretreated mesenchymal stem cells (MSCs). Leukemic cells were pretreated with vehicle or SB203580SB (20 μM) for 2 days and seeded onto MSCs that were pretreated with imatinib (5 μM) for 4 days. Scale bars, 50 μm.

### DESCRIPTION OF ILLUSTRATIVE EMBODIMENTS

Prostate Cancer

Advanced prostate adenocarcinomas enriched in stem-cell features, as well as variant androgen receptor (AR)-negative neuroendocrine/small-cell prostate cancers are difficult to treat, and account for up to 30% of prostate cancer-related deaths every year. While existing therapies for prostate cancer such as androgen deprivation therapy (ADT), destroy the bulk of the AR-positive cells within the tumor, eradicating this population eventually leads to castration-resistance, owing to the continued survival of AR^{-/lo} stem-like cells. The present disclosure overcomes challenges associated with current technologies by providing methods and compositions for treating cancer by combining inhibition of p38 MAPK with an anti-cancer therapy.

The inventors identified a critical nexus between p38MAPK signaling and the transcription-factor FOXC2 known to promote cancer stem cells and metastasis. They demonstrated that there is a direct link between PSA^{-lo} PCa cells, the EMT/CSC archetype and regulated AR expression, and established a vital role for FOXC2 in the induction and maintenance of ADT-resistant PCaSC attributes. The present disclosure demonstrates that prostate cancer cells that are insensitive to ADT, as well as high-grade/neuroendocrine prostate tumors, are characterized by elevated FOXC2, and that targeting FOXC2 using a well-tolerated p38-inhibitor restores epithelial attributes and ADT-sensitivity, and reduces the shedding of circulating tumor cells in vivo with significant shrinkage in the tumor mass. Thus, provided herein is a tangible mechanism to target the AR^{-/lo} population of prostate cancer cells with stem-like properties.

Breast Cancer

Metastatic competence is contingent upon the aberrant activation of a latent embryonic program, known as the epithelial-mesenchymal transition (EMT), which bestows stem cell properties as well as migratory and invasive capabilities upon tumor cells. The Forkhead transcription factor FOXC2 was recently identified as a downstream effector of multiple EMT programs, independent of the EMT-inducing stimulus, and as a key player linking EMT, stem cell properties and metastatic competence. As such, FOXC2 could serve as a potential target to prevent metastasis. Since FOXC2 is a transcription factor, it is difficult to target by conventional means such as small molecule inhibitors.

In the present disclosure, the inventors identified the serine/threonine specific-protein kinase p38 as a druggable upstream regulator of FOXC2 stability and function. Indeed, it was demonstrated that FOXC2 is phosphorylated at serine 367 by p38, stabilizing FOXC2 protein levels, and eliciting expression of its downstream target ZEB1. Strikingly, genetic or pharmacological inhibition of p38 decreases FOXC2 and ZEB1 protein levels, reverts EMT and selectively prevents metastasis without impacting primary tumor growth. Accordingly, inhibition of p38 impairs EMT and stem cell attributes in vitro-including migration, invadopodia formation, CD44^{high}/CD24^{low} antigenic profile and sphere-forming efficiency- and impedes tumor cell entry into the circulation from an orthotopic primary tumor site. Importantly, the phosphomimetic FOXC2(S367E) mutant is refractory to p38 inhibition in an orthotopic transplantation model, whereas the non-phosphorylatable FOXC2(S367A) mutant fails to elicit EMT and upregulate ZEB1. Collectively, it is demonstrated that FOXC2 regulates ZEB1 expression and metastasis in a p38-dependent manner, and attest to the utility of p38-inhibitors as anti-metastatic agents useful in the treatment of cancer, specifically in combination with other anti-cancer agents.

Leukemia

In further studies, the inventors determined that blocking imatinib-induced alternative survival signal transduction at any point between MSCs and leukemic cells eliminates imatinib-induced MSC-mediated drug resistance. A library of clinical compounds was screened to identify compounds that could prevent imatinib off-target effects in MSCs and sensitize leukemic cells to imatinib therapy. The present findings demonstrate that both a p38 MAPK inhibitor (e.g., SB203580) and a glucocorticoid receptor agonist (e.g., dexamethasone) eliminated tyrosine kinase inhibitor (TKI)-induced MSC-mediated survival support for BCR-ABL positive acute lymphoblastic leukemia (ALL) cells and prevented TKI resistance. Thus, embodiments of the present disclosure provide methods of treating leukemia (e.g., ALL) by administering a p38 inhibitor and/or a glucocorticoid receptor agonist in combination with a TKI.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “therapeutic benefit” used throughout this application refers to anything that promotes or enhances the well-being of the patient with respect to the medical treatment of his cancer. A list of nonexhaustive examples of this includes extension of the patient's life by any period of time; decrease or delay in the neoplastic development of the disease; decrease in hyperproliferation; reduction in tumor growth; delay of metastases; reduction in the proliferation rate of a cancer cell or tumor cell; induction of apoptosis in any treated cell or in any cell affected by a treated cell; and a decrease in pain to the patient that can be attributed to the patient's condition.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “p38 MAPK inhibitor” or “p38 inhibitor” means any compound that blocks signaling through the p38 MAP kinase pathway. In some embodiments, p38 MAPK inhibitors function by reducing the amount of p38 MAPK, inhibiting or blocking p38 MAPK activation, or inhibiting other molecules in the signaling pathway. As used herein, the term “inhibitor” includes, but is not limited to, any suitable molecule, compound, protein or fragment thereof, nucleic acid, formulation or substance that can regulate p38 MAP kinase activity.

As used herein, the term “BCR-ABL” refers to the fusion oncogene which encodes a chimeric BCR-ABL protein with a constitutively active BCR-ABL tyrosine kinase (TK) activity. In this context, protein tyrosine kinases encoded by the BCR-ABL gene can include, for example BCR-ABL p210 fusion protein (accession number: A1Z199) and BCR-ABL pi 85 fusion protein (accession number: Q13745). The term BCR-ABL is intended to be inclusive of alternative BCR-ABL gene products and also is inclusive of alternative designations such as BCR-ABL oncogene, BCR-ABL protooncogene, and BCR-ABL oncoprotein used by those skilled in the art.

As used herein, the terms “BCR-ABL tyrosine kinase inhibitor,” “BCR-ABL kinase inhibitor,” “BCR-ABL KI” and “BCR-ABL TKI” refer to any compound or agent that can inhibit BCR-ABL TK activity in an animal, in particular a mammal, for example a human. In this context, an inhibitor is understood to decrease the activity of a BCR-ABL tyrosine kinase compared to the activity in the absence of the exogenously administered compound or agent. The term is intended to include indirectly or directly acting compounds or agents. As used in the art, BCR-ABL TKI generally refers to a class of compounds which are known to inhibit BCR-ABL TK, but may further inhibit alternative signaling pathways, such as for example, Src pathway.

As used herein, the term “BCR-ABL related disorders” refers to disorders or diseases which are associated with or manifest from BCR-ABL-mediated activity, and is intended to be inclusive of mutated forms of BCR-ABL. In this context, disorders associated with BCR-ABL would benefit by direct or indirect BCR-ABL inhibition.

### II. CANCER

A. Prostate Cancer

Prostate cancer is a disease in which cancer develops in the prostate, a gland in the male reproductive system. In 2007, almost 220,000 new cases were reported, and over 27,000 deaths were attributed to this malignancy. It occurs when cells of the prostate mutate and begin to multiply out of control. These cells may spread (metastasize) from the prostate to other parts of the body, especially the bones and lymph nodes. Prostate cancer may cause pain, difficulty in urinating, erectile dysfunction and other symptoms.

Rates of prostate cancer vary widely across the world. Although the rates vary widely between countries, it is least common in South and East Asia, more common in Europe, and most common in the United States. According to the American Cancer Society, prostate cancer is least common among Asian men and most common among black men, with FIGS. for white men in-between. However, these high rates may be affected by increasing rates of detection.

Prostate cancer develops most frequently in men over fifty. This cancer can occur only in men, as the prostate is exclusively of the male reproductive tract. It is the most common type of cancer in men in the United States, where it is responsible for more male deaths than any other cancer, except lung cancer. However, many men who develop prostate cancer never have symptoms, undergo no therapy, and eventually die of other causes. Many factors, including genetics and diet, have been implicated in the development of prostate cancer.

Prostate cancer screening is an attempt to find unsuspected cancers. Screening tests may lead to more specific follow-up tests such as a biopsy, where small pieces of the prostate are removed for closer study. As of 2006 prostate cancer screening options include the digital rectal exam and the prostate specific antigen (PSA) blood test. Screening for prostate cancer is controversial because it is not clear if the benefits of screening outweigh the risks of follow-up diagnostic tests and cancer treatments.

Prostate cancer is a slow-growing cancer, very common among older men. In fact, most prostate cancers never grow to the point where they cause symptoms, and most men with prostate cancer die of other causes before prostate cancer has an impact on their lives. The PSA screening test may detect these small cancers that would never become life threatening. Doing the PSA test in these men may lead to overdiagnosis, including additional testing and treatment. Follow-up tests, such as prostate biopsy, may cause pain, bleeding and infection. Prostate cancer treatments may cause urinary incontinence and erectile dysfunction. Therefore, it is essential that the risks and benefits of diagnostic procedures and treatment be carefully considered before PSA screening.

Prostate cancer screening generally begins after age 50, but this can vary due to ethnic backgrounds. Thus, the American Academy of Family Physicians and American College of Physicians recommend the physician discuss the risks and benefits of screening and decide based on individual patient preference. Although there is no officially recommended cutoff, many health care providers stop monitoring PSA in men who are older than 75 years old because of concern that prostate cancer therapy may do more harm than good as age progresses and life expectancy decreases.

Digital rectal examination (DRE) is a procedure where the examiner inserts a gloved, lubricated finger into the rectum to check the size, shape, and texture of the prostate. Areas which are irregular, hard or lumpy need further evaluation, since they may contain cancer. Although the DRE only evaluates the back of the prostate, 85% of prostate cancers arise in this part of the prostate. Prostate cancer which can be felt on DRE is generally more advanced. The use of DRE has never been shown to prevent prostate cancer deaths when used as the only screening test.

The PSA test measures the blood level of prostate-specific antigen, an enzyme produced by the prostate. Specifically, PSA is a serine protease similar to kallikrein. Its normal function is to liquify gelatinous semen after ejaculation, allowing spermatazoa to more easily navigate through the uterine cervix.

PSA levels under 4 ng/mL (nanograms per milliliter) are generally considered normal, however in individuals below the age of 50 sometimes a cutoff of 2.5 is used for the upper limit of normal, while levels over 4 ng/mL are considered abnormal (although in men over 65 levels up to 6.5 ng/mL may be acceptable, depending upon each laboratory's reference ranges). PSA levels between 4 and 10 ng/mL indicate a risk of prostate cancer higher than normal, but the risk does not seem to rise within this six-point range. When the PSA level is above 10 ng/mL, the association with cancer becomes stronger. However, PSA is not a perfect test. Some men with prostate cancer do not have an elevated PSA, and most men with an elevated PSA do not have prostate cancer.

PSA levels can change for many reasons other than cancer. Two common causes of high PSA levels are enlargement of the prostate (benign prostatic hypertrophy (BPH)) and infection in the prostate (prostatitis). It can also be raised for 24 hours after ejaculation and several days after catheterization. PSA levels are lowered in men who use medications used to treat BPH or baldness. These medications, finasteride (marketed as Proscar or Propecia) and dutasteride (marketed as Avodart), may decrease the PSA levels by 50% or more.

Several other ways of evaluating the PSA have been developed to avoid the shortcomings of simple PSA screening. The use of age-specific reference ranges improves the sensitivity and specificity of the test. The rate of rise of the PSA over time, called the PSA velocity, has been used to evaluate men with PSA levels between 4 and 10 ng/ml, but as of 2006, it has not proven to be an effective screening test. Comparing the PSA level with the size of the prostate, as measured by ultrasound or magnetic resonance imaging, has also been studied. This comparison, called PSA density, is both costly and, as of 2006, has not proven to be an effective screening test. PSA in the blood may either be free or bound to other proteins. Measuring the amount of PSA which is free or bound may provide additional screening information, but as of 2006, questions regarding the usefulness of these measurements limit their widespread use.

When a man has symptoms of prostate cancer, or a screening test indicates an increased risk for cancer, more invasive evaluation is offered. The only test which can fully confirm the diagnosis of prostate cancer is a biopsy, the removal of small pieces of the prostate for microscopic examination. However, prior to a biopsy, several other tools may be used to gather more information about the prostate and the urinary tract. Cystoscopy shows the urinary tract from inside the bladder, using a thin, flexible camera tube inserted down the urethra. Transrectal ultrasonography creates a picture of the prostate using sound waves from a probe in the rectum.

If cancer is suspected, a biopsy is offered. During a biopsy a urologist obtains tissue samples from the prostate via the rectum. A biopsy gun inserts and removes special hollow-core needles (usually three to six on each side of the prostate) in less than a second. Prostate biopsies are routinely done on an outpatient basis and rarely require hospitalization. Fifty-five percent of men report discomfort during prostate biopsy.

The tissue samples are then examined under a microscope to determine whether cancer cells are present, and to evaluate the microscopic features of any cancer found. If cancer is present, the pathologist reports the grade of the tumor. The grade tells how much the tumor tissue differs from normal prostate tissue and suggests how fast the tumor is likely to grow. The Gleason system is used to grade prostate tumors from 2 to 10, where a Gleason score of 10 indicates the most abnormalities. The pathologist assigns a number from 1 to 5 for the most common pattern observed under the microscope, then does the same for the second most common pattern. The sum of these two numbers is the Gleason score. The Whitmore-Jewett stage is another method sometimes used. Proper grading of the tumor is critical, since the grade of the tumor is one of the major factors used to determine the treatment recommendation.

An important part of evaluating prostate cancer is determining the stage, or how far the cancer has spread. Knowing the stage helps define prognosis and is useful when selecting therapies. The most common system is the four-stage TNM system (abbreviated from Tumor/Nodes/Metastases). Its components include the size of the tumor, the number of involved lymph nodes, and the presence of any other metastases.

The most important distinction made by any staging system is whether or not the cancer is still confined to the prostate. In the TNM system, clinical T1 and T2 cancers are found only in the prostate, while T3 and T4 cancers have spread elsewhere. Several tests can be used to look for evidence of spread. These include computed tomography to evaluate spread within the pelvis, bone scans to look for spread to the bones, and endorectal coil magnetic resonance imaging to closely evaluate the prostatic capsule and the seminal vesicles. Bone scans should reveal osteoblastic appearance due to increased bone density in the areas of bone metastisis—opposite to what is found in many other cancers that metastisize.

Prostate cancer can be treated with surgery, radiation therapy, hormonal therapy, occasionally chemotherapy, proton therapy, or some combination of these. The age and underlying health of the man as well as the extent of spread, appearance under the microscope, and response of the cancer to initial treatment are important in determining the outcome of the disease. Since prostate cancer is a disease of older men, many will die of other causes before a slowly advancing prostate cancer can spread or cause symptoms. This makes treatment selection difficult. The decision whether or not to treat localized prostate cancer (a tumor that is contained within the prostate) with curative intent is a patient trade-off between the expected beneficial and harmful effects in terms of patient survival and quality of life.

Watchful waiting, also called “active surveillance,” refers to observation and regular monitoring without invasive treatment. Watchful waiting is often used when an early stage, slow-growing prostate cancer is found in an older man. Watchful waiting may also be suggested when the risks of surgery, radiation therapy, or hormonal therapy outweigh the possible benefits. Other treatments can be started if symptoms develop, or if there are signs that the cancer growth is accelerating (e.g., rapidly rising PSA, increase in Gleason score on repeat biopsy, etc.). Most men who choose watchful waiting for early stage tumors eventually have signs of tumor progression, and they may need to begin treatment within three years. Although men who choose watchful waiting avoid the risks of surgery and radiation, the risk of metastasis (spread of the cancer) may be increased. For younger men, a trial of active surveillance may not mean avoiding treatment altogether, but may reasonably allow a delay of a few years or more, during which time the quality of life impact of active treatment can be avoided. Published data to date suggest that carefully selected men will not miss a window for cure with this approach. Additional health problems that develop with advancing age during the observation period can also make it harder to undergo surgery and radiation therapy.

Clinically insignificant prostate tumors are often found by accident when a doctor incorrectly orders a biopsy not following the recommended guidelines (abnormal DRE and elevated PSA). The urologist must check that the PSA is not elevated for other reasons, prostatitis, etc. An annual biopsy is often recommended by a urologist for a patient who has selected watchful waiting when the tumor is clinically insignificant (no abnormal DRE or PSA). The tumors tiny size can be monitored this way and the patient can decide to have surgery only if the tumor enlarges which may take many years or never.

Surgical removal of the prostate, or prostatectomy, is a common treatment either for early stage prostate cancer, or for cancer which has failed to respond to radiation therapy. The most common type is radical retropubic prostatectomy, when the surgeon removes the prostate through an abdominal incision. Another type is radical perineal prostatectomy, when the surgeon removes the prostate through an incision in the perineum, the skin between the scrotum and anus. Radical prostatectomy can also be performed laparoscopically, through a series of small (1 cm) incisions in the abdomen, with or without the assistance of a surgical robot.

Radical prostatectomy is effective for tumors which have not spread beyond the prostate; cure rates depend on risk factors such as PSA level and Gleason grade. However, it may cause nerve damage that significantly alters the quality of life of the prostate cancer survivor. The most common serious complications are loss of urinary control and impotence. Reported rates of both complications vary widely depending on how they are assessed, by whom, and how long after surgery, as well as the setting (e.g., academic series vs. community-based or population-based data). Although penile sensation and the ability to achieve orgasm usually remain intact, erection and ejaculation are often impaired. Medications such as sildenafil (Viagra), tadalafil (Cialis), or vardenafil (Levitra) may restore some degree of potency. For most men with organ-confined disease, a more limited “nerve-sparing” technique may help avoid urinary incontinence and impotence.

Radical prostatectomy has traditionally been used alone when the cancer is small. In the event of positive margins or locally advanced disease found on pathology, adjuvant radiation therapy may offer improved survival. Surgery may also be offered when a cancer is not responding to radiation therapy. However, because radiation therapy causes tissue changes, prostatectomy after radiation has a higher risk of complications.

Transurethral resection of the prostate, commonly called a “TURP,” is a surgical procedure performed when the tube from the bladder to the penis (urethra) is blocked by prostate enlargement. TURP is generally for benign disease and is not meant as definitive treatment for prostate cancer. During a TURP, a small tube (cystoscope) is placed into the penis and the blocking prostate is cut away.

In metastatic disease, where cancer has spread beyond the prostate, removal of the testicles (called orchiectomy) may be done to decrease testosterone levels and control cancer growth.

Radiation therapy, also known as radiotherapy, uses ionizing radiation to kill prostate cancer cells. When absorbed in tissue, ionizing radiation such as D and x-rays damage the DNA in cells, which increases the probability of apoptosis. Two different kinds of radiation therapy are used in prostate cancer treatment: external beam radiation therapy and brachytherapy.

External beam radiation therapy uses a linear accelerator to produce high-energy x-rays which are directed in a beam towards the prostate. A technique called Intensity Modulated Radiation Therapy (IMRT) may be used to adjust the radiation beam to conform with the shape of the tumor, allowing higher doses to be given to the prostate and seminal vesicles with less damage to the bladder and rectum. External beam radiation therapy is generally given over several weeks, with daily visits to a radiation therapy center. New types of radiation therapy may have fewer side effects then traditional treatment, one of these is Tomotherapy.

Permanent implant brachytherapy is a popular treatment choice for patients with low to intermediate risk features, can be performed on an outpatient basis, and is associated with good 10-year outcomes with relatively low morbidity. It involves the placement of about 100 small “seeds” containing radioactive material (such as iodine125 or palladium103) with a needle through the skin of the perineum directly into the tumor while under spinal or general anesthetic. These seeds emit lower-energy X-rays which are only able to travel a short distance. Although the seeds eventually become inert, they remain in the prostate permanently. The risk of exposure to others from men with implanted seeds is generally accepted to be insignificant.

Radiation therapy is commonly used in prostate cancer treatment. It may be used instead of surgery for early cancers, and it may also be used in advanced stages of prostate cancer to treat painful bone metastases. Radiation treatments also can be combined with hormonal therapy for intermediate risk disease, when radiation therapy alone is less likely to cure the cancer. Some radiation oncologists combine external beam radiation and brachytherapy for intermediate to high risk situations. One study found that the combination of six months of androgen suppressive therapy combined with external beam radiation had improved survival compared to radiation alone in patients with localized prostate cancer. Others use a “triple modality” combination of external beam radiation therapy, brachytherapy, and hormonal therapy.

Less common applications for radiotherapy are when cancer is compressing the spinal cord, or sometimes after surgery, such as when cancer is found in the seminal vesicles, in the lymph nodes, outside the prostate capsule, or at the margins of the biopsy.

Radiation therapy is often offered to men whose medical problems make surgery more risky. Radiation therapy appears to cure small tumors that are confined to the prostate just about as well as surgery. However, as of 2006 some issues remain unresolved, such as whether radiation should be given to the rest of the pelvis, how much the absorbed dose should be, and whether hormonal therapy should be given at the same time.

Side effects of radiation therapy might occur after a few weeks into treatment. Both types of radiation therapy may cause diarrhea and rectal bleeding due to radiation proctitis, as well as urinary incontinence and impotence. Symptoms tend to improve over time. Men who have undergone external beam radiation therapy will have a higher risk of later developing colon cancer and bladder cancer.

Cryosurgery is another method of treating prostate cancer. It is less invasive than radical prostatectomy, and general anesthesia is less commonly used. Under ultrasound guidance, metal rods are inserted through the skin of the perineum into the prostate. Highly purified Argon gas is used to cool the rods, freezing the surrounding tissue at −196° C. (−320° F.). As the water within the prostate cells freeze, the cells die. The urethra is protected from freezing by a catheter filled with warm liquid. Cryosurgery generally causes fewer problems with urinary control than other treatments, but impotence occurs up to ninety percent of the time. When used as the initial treatment for prostate cancer and in the hands of an experienced cryosurgeon, cryosurgery has a 10 year biochemical disease free rate superior to all other treatments including radical prostatectomy and any form of radiation Cryosurgery has also been demonstrated to be superior to radical prostatectomy for recurrent cancer following radiation therapy.

Hormonal therapy uses medications or surgery to block prostate cancer cells from getting dihydrotestosterone (DHT), a hormone produced in the prostate and required for the growth and spread of most prostate cancer cells. Blocking DHT often causes prostate cancer to stop growing and even shrink. However, hormonal therapy rarely cures prostate cancer because cancers which initially respond to hormonal therapy typically become resistant after one to two years. Hormonal therapy is therefore usually used when cancer has spread from the prostate. It may also be given to certain men undergoing radiation therapy or surgery to help prevent return of their cancer.

Hormonal therapy for prostate cancer targets the pathways the body uses to produce DHT. A feedback loop involving the testicles, the hypothalamus, and the pituitary, adrenal, and prostate glands controls the blood levels of DHT. First, low blood levels of DHT stimulate the hypothalamus to produce gonadotropin releasing hormone (GnRH). GnRH then stimulates the pituitary gland to produce luteinizing hormone (LH), and LH stimulates the testicles to produce testosterone. Finally, testosterone from the testicles and dehydroepiandrosterone from the adrenal glands stimulate the prostate to produce more DHT. Hormonal therapy can decrease levels of DHT by interrupting this pathway at any point.

There are several forms of hormonal therapy. Orchiectomy is surgery to remove the testicles. Because the testicles make most of the body's testosterone, after orchiectomy testosterone levels drop. Now the prostate not only lacks the testosterone stimulus to produce DHT, but also it does not have enough testosterone to transform into DHT.

Anti-androgens are medications such as flutamide, bicalutamide, nilutamide, and cyproterone acetate which directly block the actions of testosterone and DHT within prostate cancer cells.

Medications which block the production of adrenal androgens such as DHEA include ketoconazole and aminoglutethimide. Because the adrenal glands only make about 5% of the body's androgens, these medications are generally used only in combination with other methods that can block the 95% of androgens made by the testicles. These combined methods are called total androgen blockade (TAB). TAB can also be achieved using antiandrogens.

GnRH action can be interrupted in one of two ways. GnRH antagonists suppress the production of LH directly, while GnRH agonists suppress LH through the process of downregulation after an initial stimulation effect. Abarelix is an example of a GnRH antagonist, while the GnRH agonists include leuprolide, goserelin, triptorelin, and buserelin. Initially, GnRH agonists increase the production of LH. However, because the constant supply of the medication does not match the body's natural production rhythm, production of both LH and GnRH decreases after a few weeks.

B. Breast Cancer

1. Background

Breast cancer is a cancer that starts in the breast, usually in the inner lining of the milk ducts or lobules. There are different types of breast cancer, with different stages (spread), aggressiveness, and genetic makeup. With best treatment, 10-year disease-free survival varies from 98% to 10%. Treatment is selected from surgery, drugs (chemotherapy), and radiation. In the United States, there were 216,000 cases of invasive breast cancer and 40,000 deaths in 2004. Worldwide, breast cancer is the second most common type of cancer after lung cancer (10.4% of all cancer incidence, both sexes counted) and the fifth most common cause of cancer death. In 2004, breast cancer caused 519,000 deaths worldwide (7% of cancer deaths; almost 1% of all deaths). Breast cancer is about 100 times as frequent among women as among men, but survival rates are equal in both sexes.

Some breast cancers require the hormones estrogen and progesterone to grow, and have receptors for those hormones. After surgery those cancers are treated with drugs that interfere with those hormones, usually tamoxifen, and with drugs that shut off the production of estrogen in the ovaries or elsewhere; this may damage the ovaries and end fertility. After surgery, low-risk, hormone-sensitive breast cancers may be treated with hormone therapy and radiation alone. Breast cancers without hormone receptors, or which have spread to the lymph nodes in the armpits, or which express certain genetic characteristics, are higher-risk, and are treated more aggressively. One standard regimen, popular in the U.S., is cyclophosphamide plus doxorubicin (Adriamycin), known as CA; these drugs damage DNA in the cancer, but also in fast-growing normal cells where they cause serious side effects. Sometimes a taxane drug, such as docetaxel, is added, and the regime is then known as CAT; taxane attacks the microtubules in cancer cells. An equivalent treatment, popular in Europe, is cyclophosphamide, methotrexate, and fluorouracil (CMF). Monoclonal antibodies, such as trastuzumab (Herceptin), are used for cancer cells that have the HER2 mutation. Radiation is usually added to the surgical bed to control cancer cells that were missed by the surgery, which usually extends survival, although radiation exposure to the heart may cause damage and heart failure in the following years.

2. Symptoms

The first symptom, or subjective sign, of breast cancer is typically a lump that feels different from the surrounding breast tissue. According to the The Merck Manual, more than 80% of breast cancer cases are discovered when the woman feels a lump. According to the American Cancer Society, the first medical sign, or objective indication of breast cancer as detected by a physician, is discovered by mammogram. Lumps found in lymph nodes located in the armpits can also indicate breast cancer. Indications of breast cancer other than a lump may include changes in breast size or shape, skin dimpling, nipple inversion, or spontaneous single-nipple discharge. Pain (“mastodynia”) is an unreliable tool in determining the presence or absence of breast cancer, but may be indicative of other breast health issues.

When breast cancer cells invade the dermal lymphatics—small lymph vessels in the skin of the breast—its presentation can resemble skin inflammation and thus is known as inflammatory breast cancer (IBC). Symptoms of inflammatory breast cancer include pain, swelling, warmth and redness throughout the breast, as well as an orange-peel texture to the skin referred to as “peau d'orange.” Another reported symptom complex of breast cancer is Paget's disease of the breast. This syndrome presents as eczematoid skin changes such as redness and mild flaking of the nipple skin. As Paget's advances, symptoms may include tingling, itching, increased sensitivity, burning, and pain. There may also be discharge from the nipple. Approximately half of women diagnosed with Paget's also have a lump in the breast.

Occasionally, breast cancer presents as metastatic disease, that is, cancer that has spread beyond the original organ. Metastatic breast cancer will cause symptoms that depend on the location of metastasis. Common sites of metastasis include bone, liver, lung and brain. Unexplained weight loss can occasionally herald an occult breast cancer, as can symptoms of fevers or chills. Bone or joint pains can sometimes be manifestations of metastatic breast cancer, as can jaundice or neurological symptoms. These symptoms are “non-specific,” meaning they can also be manifestations of many other illnesses.

3. Risk Factors

The primary risk factors that have been identified are sex, age, childbearing, hormones, a high-fat diet, alcohol intake, obesity, and environmental factors such as tobacco use, radiation and shiftwork. No etiology is known for 95% of breast cancer cases, while approximately 5% of new breast cancers are attributable to hereditary syndromes. In particular, carriers of the breast cancer susceptibility genes, BRCA1 and BRCA2, are at a 30-40% increased risk for breast and ovarian cancer, depending on in which portion of the protein the mutation occurs. Experts believe that 95% of inherited breast cancer can be traced to one of these two genes. Hereditary breast cancers can take the form of a site-specific hereditary breast cancer—cancers affecting the breast only—or breast-ovarian and other cancer syndromes. Breast cancer can be inherited both from female and male relatives.

4. Subtypes

Breast cancer subtypes are typically categorized on an immunohistochemical basis. Subtype definitions are generally as follows:

normal (ER+, PR+, HER2+, cytokeratin 5/6+, and HER1+)

luminal A (ER+ and/or PR+, HER2−)

luminal B (ER+ and/or PR+, HER2+)

triple-negative (ER−, PR−, HER2−)

HER2+/ER− (ER−, PR−, and HER2+)

unclassified (ER−, PR−, HER2−, cytokeratin 5/6−, and HER1−)

In the case of triple-negative breast cancer cells, the cancer's growth is not driven by estrogen or progesterone, or by growth signals coming from the HER2 protein. By the same token, such cancer cells do not respond to hormonal therapy, such as tamoxifen or aromatase inhibitors, or therapies that target HER2 receptors, such as Herceptin®. About 10-20% of breast cancers are found to be triple-negative. It is important to identify these types of cancer so that one can avoid costly and toxic effects of therapies that are unlike to succeed, and to focus on treatments that can be used to treat triple-negative breast cancer. Like other forms of breast cancer, triple-negative breast cancer can be treated with surgery, radiation therapy, and/or chemotherapy. One particularly promosing approach is “neoadjuvant” therapy, where chemo- and/or radiotherapy is provided prior to sugery. Another drug therapy is the use of poly (ADP-ribose) polymerase, or PARP inhibitors.

5. Traditional Screening and Diagnosis

While screening techniques discussed above are useful in determining the possibility of cancer, a further testing is necessary to confirm whether a lump detected on screening is cancer, as opposed to a benign alternative such as a simple cyst. In a clinical setting, breast cancer is commonly diagnosed using a “triple test” of clinical breast examination (breast examination by a trained medical practitioner), mammography, and fine needle aspiration cytology. Both mammography and clinical breast exam, also used for screening, can indicate an approximate likelihood that a lump is cancer, and may also identify any other lesions. Fine Needle Aspiration and Cytology (FNAC), performed as an outpatient procedure using local anaesthetic, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy. Other options for biopsy include core biopsy, where a section of the breast lump is removed, and an excisional biopsy, where the entire lump is removed.

Breast cancer screening is an attempt to find cancer in otherwise healthy individuals. The most common screening method for women is a combination of x-ray mammography and clinical breast exam. In women at higher than normal risk, such as those with a strong family history of cancer, additional tools may include genetic testing or breast Magnetic Resonance Imaging.

In addition vacuum-assisted breast biopsy (VAB) may help diagnose breast cancer among patients with a mammographically detected breast in women according to a systematic review. In this study, summary estimates for vacuum assisted breast biopsy in diagnosis of breast cancer were as follows sensitivity was 98.1% with 95% CI=0.972-0.987 and specificity was 100% with 95% CI=0.997-0.999. However underestimate rates of atypical ductal hyperplasia (ADH) and ductal carcinoma in situ (DCIS) were 20.9% with 95% CI=0.177-0.245 and 11.2% with 95% CI=0.098-0.128 respectively.

Breast cancer screening refers to testing otherwise-healthy women for breast cancer in an attempt to achieve an earlier diagnosis. The assumption is that early detection will improve outcomes. A number of screening test have been employed including: clinical and self breast exams, mammography, genetic screening, ultrasound, and magnetic resonance imaging.

A clinical or self breast exam involves feeling the breast for lumps or other abnormalities. Research evidence does not support the effectiveness of either type of breast exam, because by the time a lump is large enough to be found it is likely to have been growing for several years and will soon be large enough to be found without an exam. Mammographic screening for breast cancer uses x-rays to examine the breast for any uncharacteristic masses or lumps. In women at high risk, such as those with a strong family history of cancer, mammography screening is recommended at an earlier age and additional testing may include genetic screening that tests for the BRCA genes and/or magnetic resonance imaging.

Breast self-examination was a form of screening that was heavily advocated in the past, but has since fallen into disfavour since several large studies have shown that it does not have a survival benefit for women and often causes considerably anxiety. This is thought to be because cancers that could be detected tended to be at a relatively advanced stage already, whereas other methods push to identify the cancer at an earlier stage where curative treatment is more often possible.

X-ray mammography uses x-rays to examine the breast for any uncharacteristic masses or lumps. Regular mammograms are recommended in several countries in women over a certain age as a screening tool.

Genetic testing for breast cancer typically involves testing for mutations in the BRCA genes. This is not generally a recommended technique except for those at elevated risk for breast cancer.

6. Treatments

Breast cancer is sometimes treated first with surgery, and then with chemotherapy, radiation, or both. Treatments are given with increasing aggressiveness according to the prognosis and risk of recurrence. Stage 1 cancers (and DCIS) have an excellent prognosis and are generally treated with lumpectomy with or without chemotherapy or radiation. Although the aggressive HER2+ cancers should also be treated with the trastuzumab (Herceptin) regime. Stage 2 and 3 cancers with a progressively poorer prognosis and greater risk of recurrence are generally treated with surgery (lumpectomy or mastectomy with or without lymph node removal), radiation (sometimes) and chemotherapy (plus trastuzumab for HER2+ cancers). Stage 4, metastatic cancer, (i.e., spread to distant sites) is not curable and is managed by various combinations of all treatments from surgery, radiation, chemotherapy and targeted therapies. These treatments increase the median survival time of stage 4 breast cancer by about 6 months.

The mainstay of breast cancer treatment is surgery when the tumor is localized, with possible adjuvant hormonal therapy (with tamoxifen or an aromatase inhibitor), chemotherapy, and/or radiotherapy. At present, the treatment recommendations after surgery (adjuvant therapy) follow a pattern. Depending on clinical criteria (age, type of cancer, size, metastasis) patients are roughly divided into high risk and low risk cases, with each risk category following different rules for therapy. Treatment possibilities include radiation therapy, chemotherapy, hormone therapy, and immune therapy.

Targeted cancer therapies are treatments that target specific characteristics of cancer cells, such as a protein that allows the cancer cells to grow in a rapid or abnormal way. Targeted therapies are generally less likely than chemotherapy to harm normal, healthy cells. Some targeted therapies are antibodies that work like the antibodies made naturally by one's immune system. These types of targeted therapies are sometimes called immune-targeted therapies.

There are currently 3 targeted therapies doctors use to treat breast cancer. Herceptin® (trastuzumab) works against HER2-positive breast cancers by blocking the ability of the cancer cells to receive chemical signals that tell the cells to grow. Tykerb® (lapatinib) works against HER2-positive breast cancers by blocking certain proteins that can cause uncontrolled cell growth. Avastin® (bevacizumab) works by blocking the growth of new blood vessels that cancer cells depend on to grow and function.

Hormonal (anti-estrogen) therapy works against hormone-receptor-positive breast cancer in two ways: first, by lowering the amount of the hormone estrogen in the body, and second, by blocking the action of estrogen in the body. Most of the estrogen in women's bodies is made by the ovaries. Estrogen makes hormone-receptor-positive breast cancers grow. So reducing the amount of estrogen or blocking its action can help shrink hormone-receptor-positive breast cancers and reduce the risk of hormone-receptor-positive breast cancers coming back (recurring). Hormonal therapy medicines are not effective against hormone-receptor-negative breast cancers.

There are several types of hormonal therapy medicines, including aromatase inhibitors, selective estrogen receptor modulators, and estrogen receptor downregulators. In some cases, the ovaries and fallopian tubes may be surgically removed to treat hormone-receptor-positive breast cancer or as a preventive measure for women at very high risk of breast cancer. The ovaries also may be shut down temporarily using medication.

In planning treatment, doctors can also use PCR tests like Oncotype DX or microarray tests that predict breast cancer recurrence risk based on gene expression. In February 2007, the first breast cancer predictor test won formal approval from the Food and Drug Administration. This is a new gene test to help predict whether women with early-stage breast cancer will relapse in 5 or 10 years, this could help influence how aggressively the initial tumor is treated.

Radiation therapy is also used to help destroy cancer cells that may linger after surgery. Radiation can reduce the risk of recurrence by 50-66% when delivered in the correct dose.

C. Leukemia

Leukemia is a group of cancers that usually begin in the bone marrow and result in high numbers of abnormal white blood cells. These white blood cells are not fully developed and are called blasts or leukemia cells. Symptoms may include bleeding and bruising problems, feeling tired, fever, and an increased risk of infections. These symptoms occur due to a lack of normal blood cells. Diagnosis is typically made by blood tests or bone marrow biopsy.

The exact cause of leukemia is unknown. Different kinds of leukemia are believed to have different causes. Both inherited and environmental (non-inherited) factors are believed to be involved. Risk factors include smoking, ionizing radiation, some chemicals (such as benzene), prior chemotherapy, and Down syndrome. People with a family history of leukemia are also at higher risk. [5] There are four main types of leukemia—acute lymphoblastic leukemia (ALL), acute myeloid leukemia (AML), chronic lymphocytic leukemia (CLL) and chronic myeloid leukemia (CML)—as well as a number of less common types. Leukemias and lymphomas both belong to a broader group of tumors that affect the blood, bone marrow, and lymphoid system, known as tumors of the hematopoietic and lymphoid tissues.

Treatment may involve some combination of chemotherapy, radiation therapy, targeted therapy, and bone marrow transplant, in addition to supportive care and palliative care as needed. Certain types of leukemia may be managed with watchful waiting. The success of treatment depends on the type of leukemia and the age of the person. Outcomes have improved in the developed world. The average five-year survival rate is 57% in the United States. In children under 15, the five-year survival rate is greater than 60 to 85%, depending on the type of leukemia. In children with acute leukemia who are cancer-free after five years, the cancer is unlikely to return.

The Philadelphia chromosome or Philadelphia translocation is a specific genetic abnormality in chromosome 22 of leukemia cancer cells (e.g., CML, AML, and ALL cells). This chromosome is defective and unusually short because of reciprocal translocation of genetic material between chromosome 9 and chromosome 22, and contains a fusion gene called BCR-ABL1. This gene is the ABL1 gene of chromosome 9 juxtaposed onto the BCR gene of chromosome 22, coding for a hybrid protein: a tyrosine kinase signalling protein that is “always on”, causing the cell to divide uncontrollably.

### III. METHODS OF TREATMENT

In certain embodiments, the methods described herein include the administration of a p38 MAPK inhibitor for the treatment of cancer. Examples of cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is prostate cancer, such as androgen-independent, castration-resistant prostate cancer.

In some embodiments, the present disclosure provides a method of treating cancer in a subject comprising administering to the subject a p38 MAPK inhibitor, and an anti-cancer therapy, in an amount effective to treat, wherein the subject is identified as having cancer cells that express an elevated level of FOXC2 relative to a reference level. In some aspects, the subject is a human subject. In particular aspects, the cancer is prostate cancer.

In some embodiments, the methods described herein include the administration of a p38 MAPK inhibitor for the treatment of a cancer in a subject, specifically a metastatic cancer. In certain aspects, the p38 MAPK inhibitor is administered in combination with at least one anti-cancer treatment. Examples of metastatic cancers contemplated for treatment include lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is metastatic breast cancer.

In a further embodiment, there is provided a method for treating BCR-ABL related disorders with a combination of a p38 MAPK inhibitor, glucocorticoid receptor agonist, and/or a tyrosine kinase receptor (TKI). The BCR-ABL related disorder may be a Philadelphia chromosome positive leukemia. In a further aspect, the BCR-ABL dysfunction is a mutation of the BCR-ABL gene. Examples of BCR ABL related disorders include cancers such as leukemias, lymphomas, and solid tumors. In a further aspect, the cancer is selected from leukemia and gastrointestinal stroma tumor. In particular aspects, the leukemia is chronic myeloid leukemia (CML), acute lymphoblastic leukemia (ALL), or Philadelphia chromosome positive acute lymphoblastic leukemia (Ph+ALL). It can be appreciated that additional cancers, such as those associated with tyrosine kinase dysfunction, may benefit from using the present invention, including, for example, carcinomas, colon, kidney, liver, lung, pancreas, stomach, thyroid, testis, testicular seminomas, squamous cell carcinoma, and other hematologic tumors. In some aspects, the BCR-ABL tyrosine kinase inhibitor is selected from imatinib, dasatinib, nilotinib, bosutinib, ponatinib, bafetinib, saracatinib, tozasertib and rebastinib. In particular aspects, the BCR-ABL TKI is imatinib or dasatinib. Exemplary glucocorticoid receptor agonists are dexamethasone, cortisol, cortisone, prednisolone, prednisone, methylprednisolone, trimcinolone, hydrocortisone, and corticosterone

In some embodiments, the individual has cancer that is resistant (has been demonstrated to be resistant) to one or more anti-cancer therapies. In some embodiments, resistance to anti-cancer therapy includes recurrence of cancer or refractory cancer. Recurrence may refer to the reappearance of cancer, in the original site or a new site, after treatment. In some embodiments, resistance to anti-cancer therapy includes progression of the cancer during treatment with the anti-cancer therapy. In some embodiments, the cancer is at early stage or at late stage.

The efficacy of any of the methods described herein may be tested in various models known in the art, such as clinical or pre-clinical models. Suitable pre-clinical models are exemplified herein and further may include without limitation ID8 ovarian cancer, GEM models, B16 melanoma, RENCA renal cell cancer, CT26 colorectal cancer, MC38 colorectal cancer, and Cloudman melanoma models of cancer.

It is contemplated that the particular p38 inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

Four p38 MAPK isoforms (alpha, beta, gamma and delta respectively) have been identified, each displaying a tissue-specific expression pattern. The p38 MAPK alpha and beta isoforms are ubiquitously expressed throughout the body and are found in many different cell types. The p38 MAPK alpha and beta isoforms are inhibited by certain known small molecule p38 MAPK inhibitors.

The p38 MAPK inhibitor can affect a single p38 MAP kinase isoform (e.g., p38α, p38β, p38γ or p38δ), more than one isoform, or all isoforms of p38 MAP kinase. In a particular embodiment, the inhibitor regulates the a isoform of p38 MAP kinase.

Specific examples of p38 MAPK inhibitors for use in the present methods and compositions include but are not limited to SB203580 (4-(4-Fluorophenyl)-2-(4-methylsulfinylphenyl)-5-(4-pyridyl) 1H-imidazole); SB202190 (4-(4-fluorophenyl)-2-(4-hydroxyphenyl)-5-(4-pyridyl)-1H-imidazole); SB 220025; N-(3-tert-butyl-1-methyl-5-pyrazolyl)-N′-(4-(4-pyridinylmethyl)phenyl)urea; RPR 200765A; UX-745; UX-702; UX-850; SC10-469; RWJ-67657 (RW Johnson Pharmaceutical Research Institute); RDP-58 (SangStat Medical Corp.; acquired by Genzyme Corp.); Scios-323 (SCIO 323; Scios Inc.); Scios-469 (SCIO-469; Scios Inc.); MKK3/MKK6 inhibitors (Signal Research Division); p38/MEK modulators (Signal Research Division); SB-210313 analogs; SB-238039; HEP-689 (SB 235699); SB-239063; SB-239065; SB-242235 (SmithKline Beecham Pharmaceuticals); VX-702 and VX-745 (Vertex Pharmaceuticals Inc.); AMG-548 (Amgen Inc.); Astex p38 kinase inhibitors (Astex Technology Ltd.); RPR-200765 analogs (Aventis SA); Bayer p38 kinase inhibitors (Bayer Corp.); BIRB-796 (Boehringer Ingelheim Pharmaceuticals Inc.); Celltech p38 MAP kinase inhibitor (Celltech Group plc.); FR-167653 (Fujisawa Pharmaceutical Co. Ltd.); SB-681323 and SB-281832 (GlaxoSmithKline plc); LEO Pharmaceuticals MAP kinase inhibitors (LEO Pharma A/S); Merck Co. p38 MAP kinase inhibitors (Merck research Laboratories); SC-040 and SC-XX906 (Monsanto Co.); adenosine A3 antagonists (Novartis AG); p38 MAP kinase inhibitors (Novartis Pharma AG); CNI-1493 (Picower Institute for Medical Research); RPR-200765A (Rhone-Poulenc Rorer Ltd.); and Roche p38 MAP kinase inhibitors (e.g., R03201195 and R04402257; Roche Bioscience). See, e.g., Roux, et al., Microbiology and Molecular Biology Reviews 68(2):320-344 (2004); Engelman, et al., Journal of Biological Chemistry 273(48):32111-32120 (1998); Jackson, et al., Journal of Pharmacology and Experimental Therapeutics 284(2):687-692 (1998); Kramer, et al., Journal of Biological Chemistry 271(44):27723-27729 (1996); and Menko, et al., US20080193504.

Additional inhibitors of p38 include but are not limited to 1,5-diaryl-substituted pyrazole and substituted pyrazole compounds (U.S. Pat. Nos. 6,509,361 and 6,335,336); substituted pyridyl compounds (US20030139462); quinazoline derivatives (U.S. Pat. Nos. 6,541,477, 6,184,226, 6,509,363 and 6,635,644); aryl ureas and heteroaryl analogues (U.S. Pat. No. 6,344,476); heterocyclic ureas (WO1999/32110); other urea compounds (WO1999/32463, WO1998/52558, WO1999/00357 and WO1999/58502); and substituted imidazole compounds and substituted triazole compounds (U.S. Pat. No. 6,560,871 and U.S. Pat. No. 6,599,910). Additional compounds are described in published PCT application WO 96/21452, WO 96/40143, WO 97/25046, WO 97/35856, WO 98/25619, WO 98/56377, WO 98/57966, WO 99/32110, WO 99/32121, WO 99/32463, WO 99/61440, WO 99/64400, WO 00/10563, WO 00/17204, WO 00/19824, WO 00/41698, WO 00/64422, WO 00/71535, WO 01/38324, WO 01/64679, WO 01/66539, and WO 01/66540, each of which is herein incorporated by reference in their entirety. Additional guidance regarding p38 MAPK inhibitory compounds is found in U.S. patent application Ser. No. 09/575,060 (now U.S. Pat. No. 6,867,209), Ser. No. 10/157,048 (now U.S. Pat. No. 6,864,260), Ser. Nos. 10/146,703, 10/156,997, and 10/156,996, all of which are hereby incorporated by reference in their entirety.

If acting on p38 MAP kinase directly, in one embodiment the inhibitor can exhibit an IC50 value of about 5 μM or less, such as about 500 mM or less, such as about 100 nM or less. In a related embodiment, the inhibitor should exhibit an IC50 value relative to the p38α MAP kinase isoform that is about ten-fold less than that observed when the same inhibitor is tested against other p38 MAP kinase isoforms in a comparable assay.

In some embodiments, the p38 inhibitor is administered intravenously, intramuscularly, subcutaneously, topically, orally, transdermally, intraperitoneally, intraorbitally, by implantation, by inhalation, intrathecally, intraventricularly, or intranasally. An effective amount of the p38 inhibitor may be administered for prevention or treatment of disease. The appropriate dosage of p38 inhibitor may be determined based on the type of disease to be treated, severity and course of the disease, the clinical condition of the individual, the individual's clinical history and response to the treatment, and the discretion of the attending physician.

For example, the therapeutically effective amount of the p38 inhibitor that is administered to a human will be in the range of about 0.01 to about 50 mg/kg of patient body weight whether by one or more administrations. In some embodiments, the compound is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the compound is administered at 15 mg/kg. However, other dosage regimens may be useful. In one embodiment, p38 MAPK inhibitor described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

A. Combination Therapy

The combination therapy may be administered in any suitable manner known in the art. In some embodiments, the methods described herein include the administration of a p38 inhibitor in combination with at least one anti-cancer treatment for the treatment of cancer in a subject.

For example, a p38 inhibitor and anti-cancer agent may be administered sequentially (at different times) or concurrently (at the same time). In some embodiments, the p38 inhibitor is in a separate composition as the anti-cancer agent. In some embodiments, the p38 inhibitor is in the same composition as the anti-cancer agent.

The efficacy of any of the methods described herein may be tested in various models known in the art, such as clinical or pre-clinical models. Suitable pre-clinical models are exemplified herein and further may include without limitation ID8 ovarian cancer, GEM models, B16 melanoma, RENCA renal cell cancer, CT26 colorectal cancer, MC38 colorectal cancer, and Cloudman melanoma models of cancer.

The p38 inhibitor and anti-cancer agent may be administered by the same route of administration or by different routes of administration. In some embodiments, the p38 inhibitor is administered intravenously, intramuscularly, subcutaneously, topically, orally, transdermally, intraperitoneally, intraorbitally, by implantation, by inhalation, intrathecally, intraventricularly, or intranasally. In some embodiments, the anti-cancer agent is administered intravenously, intramuscularly, subcutaneously, topically, orally, transdermally, intraperitoneally, intraorbitally, by implantation, by inhalation, intrathecally, intraventricularly, or intranasally. An effective amount of the p38 inhibitor and anti-cancer agent may be administered for prevention or treatment of disease. The appropriate dosage of p38 inhibitor and anti-cancer agent may be determined based on the type of disease to be treated, severity and course of the disease, the clinical condition of the individual, the individual's clinical history and response to the treatment, and the discretion of the attending physician. In some embodiments, combination treatment with p38 inhibitor and anti-cancer agent are synergistic, whereby an efficacious dose of a p38 inhibitor in the combination is reduced relative to efficacious dose of at the least one anti-cancer agent as a single agent.

For example, the therapeutically effective amount of the p38 inhibitor and anti-cancer agent that is administered to a human will be in the range of about 0.01 to about 50 mg/kg of patient body weight whether by one or more administrations. In some embodiments, the compound used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In some embodiments, the compound is administered at 15 mg/kg. However, other dosage regimens may be useful. In one embodiment, the p38 MAPK inhibitor described herein is administered to a human at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

Intratumoral injection, or injection into the tumor vasculature is specifically contemplated for discrete, solid, accessible tumors. Local, regional or systemic administration also may be appropriate. For tumors of >4 cm, the volume to be administered will be about 4-10 ml (in particular 10 ml), while for tumors of <4 cm, a volume of about 1-3 ml will be used (in particular 3 ml). Multiple injections delivered as single dose comprise about 0.1 to about 0.5 ml volumes.

An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the anti-cancer peptide or nanoparticle complex and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the anti-cancer peptide or nanoparticle complex and the other includes the second agent(s). In particular embodiments, an anti-cancer peptide can be one agent, and an anti-cancer nanoparticle complex can be the other agent.

Various combinations may be employed, where the p38 inhibitor is “A” and the one or more anti-cancer agents, such as radiotherapy or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In certain embodiments, administration of the combination therapy of the present embodiments to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies. In some aspects a p38 MAPK inhibitor is administered (or formulated) in conjunction with a chemotherapeutic agent. For example, in some aspects the chemotherapeutic agent is a protein kinase inhibitor such as a EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein tansferase inhibitors, transplatinum, and pharmaceutically acceptable salts, acids or derivatives of any of the above. In certain embodiments, the compositions provided herein may be used in combination with gefitinib. In other embodiments, the present embodiments may be practiced in combination with Gleevac (e.g., from about 400 to about 800 mg/day of Gleevac may be administered to a patient). In certain embodiments, one or more chemotherapeutic may be used in combination with the compositions provided herein.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic composition and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a TUSC2 therapy of the present embodiments. The general approach for combined therapy is discussed below. Generally, the tumor cell must bear some marker that is amenable to targeting, i.e., is not present on the majority of other cells. Many tumor markers exist and any of these may be suitable for targeting in the context of the present embodiments. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Gene Therapy

In yet another embodiment, the secondary treatment is a gene therapy in which a therapeutic polynucleotide is administered before, after, or at the same time as the therapeutic composition. Viral vectors for the expression of a gene product are well known in the art, and include such eukaryotic expression systems as adenoviruses, adeno-associated viruses, retroviruses, herpesviruses, lentiviruses, poxviruses including vaccinia viruses, and papiloma viruses, including SV40. Alternatively, the administration of expression constructs can be accomplished with lipid based vectors such as liposomes or DOTAP:cholesterol vesicles. All of these methods are well known in the art (see, e.g. Sambrook et al., 1989; Ausubel et al., 1998; Ausubel, 1996).

Delivery of a vector encoding one of the following gene products will have a combined anti-hyperproliferative effect on target tissues. A variety of proteins are encompassed within the present embodiments, some of which are described below.

i. Inhibitors of Cellular Proliferation

As noted above, the tumor suppressor oncogenes function to inhibit excessive cellular proliferation. The inactivation of these genes destroys their inhibitory activity, resulting in unregulated proliferation.

Genes that may be employed as secondary treatment in accordance with the present embodiments include p53, p16, Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors), MCC and other genes listed in Table IV.

ii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, Proc. Nat'l. Acad. Sci. USA, 82(21):7439-43, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatments provided herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present embodiments may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyperproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

B. Pharmaceutical Formulations

Pharmaceutical compositions provided herein comprise an effective amount of a p38 MAPK inhibitor. In other embodiments, pharmaceutical compositions provided herein comprise an effective amount of one or more anti-cancer agents and a p38 MAPK inhibitor. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least a p38 MAPK inhibitor and optionally an anti-cancer agent will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

In certain embodiments, the pharmaceutical composition may comprise different types of carriers depending on whether it is to be administered in solid, liquid or aerosol form, and whether it need to be sterile for such routes of administration as injection. In certain embodiments, pharmaceutical compositions provided herein can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In certain embodiments, the pharmaceutical composition is administered intraperitoneally. In further embodiments, the pharmaceutical composition is administered intraperitoneally to treat a cancer (e.g., a cancerous tumor). For example, the pharmaceutical composition may be administered intraperitoneally to treat gastrointestinal cancer. In certain embodiments it may be disirable to administer the pharmaceutical composition into or near a tumor.

In certain preferred embodiments, the pharmaceutical composition is administered orally to treat a cancer (e.g., a gastrointestinal cancer).

In certain embodiments, the actual dosage amount of a composition administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 15 microgram/kg/body weight, about 20 microgram/kg/body weight, about 25 microgram/kg/body weight, about 30 microgram/kg/body weight, about 35 microgram/kg/body weight, about 0.04 milligram/kg/body weight, about 0.05 milligram/kg/body weight, about 0.06 milligram/kg/body weight, about 0.07 milligram/kg/body weight, about 0.08 milligram/kg/body weight, about 0.09 milligram/kg/body weight, about 0.1 milligram/kg/body weight, about 0.2 milligram/kg/body weight, to about 0.5 mg/kg/body weight or more per administration, and any range derivable therein. In non-limiting examples of a derivable range from the numbers listed herein, a range of about 0.01 mg/kg/body weight to about 0.1 mg/kg/body weight, about 0.04 microgram/kg/body weight to about 0.08 milligram/kg/body weight, etc., can be administered, based on the numbers described above.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present embodiments. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Inhibition of FOXC2 Restores Epithelial Phenotype and Drug-Sensitivity in Prostate Cancer Cells with Stem-Like Properties

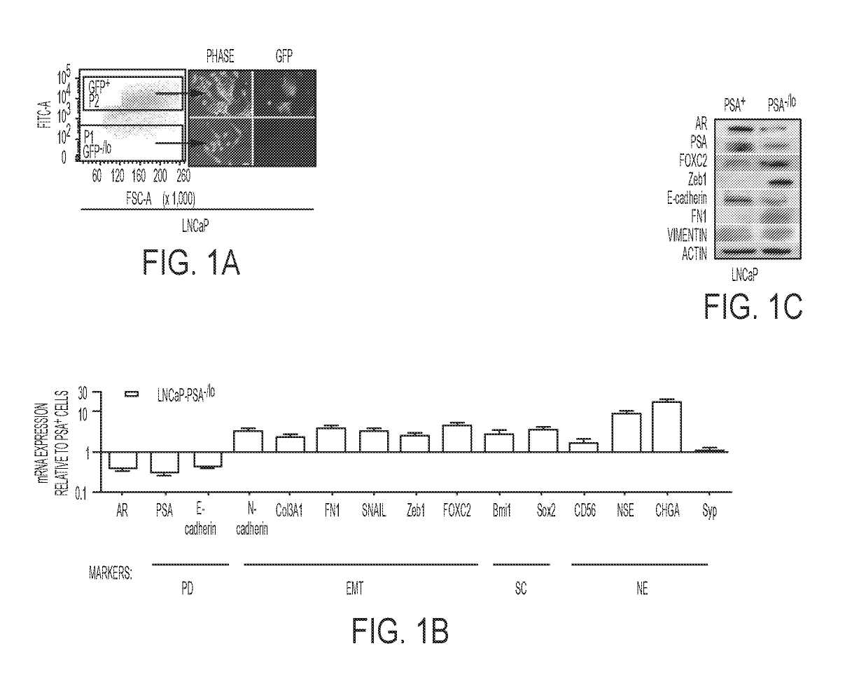

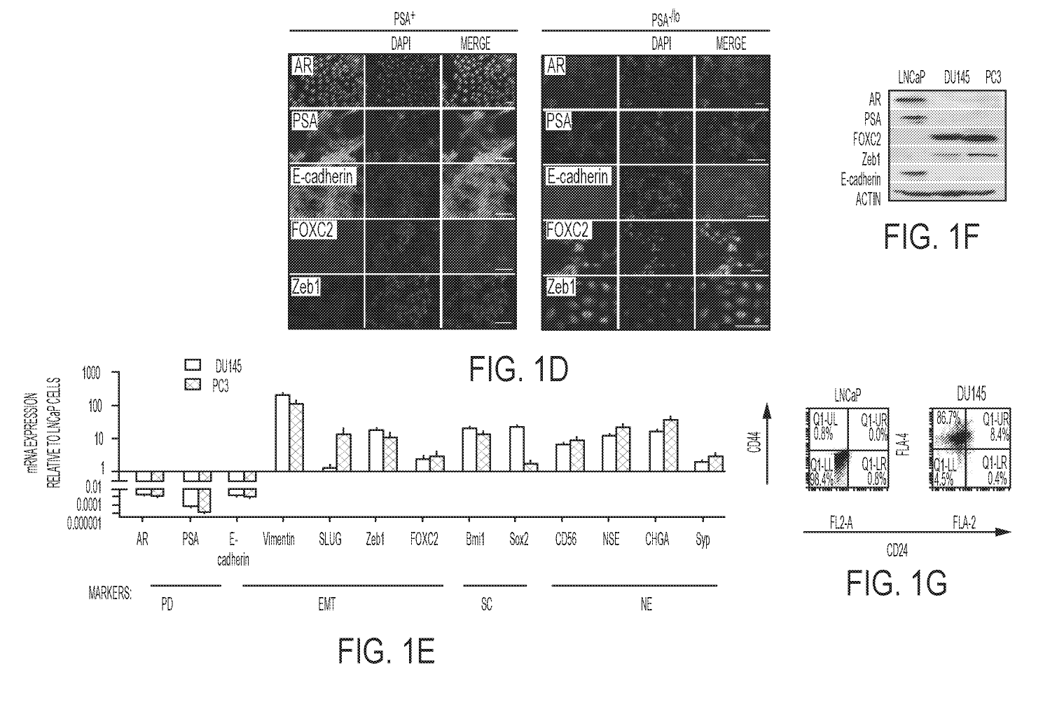

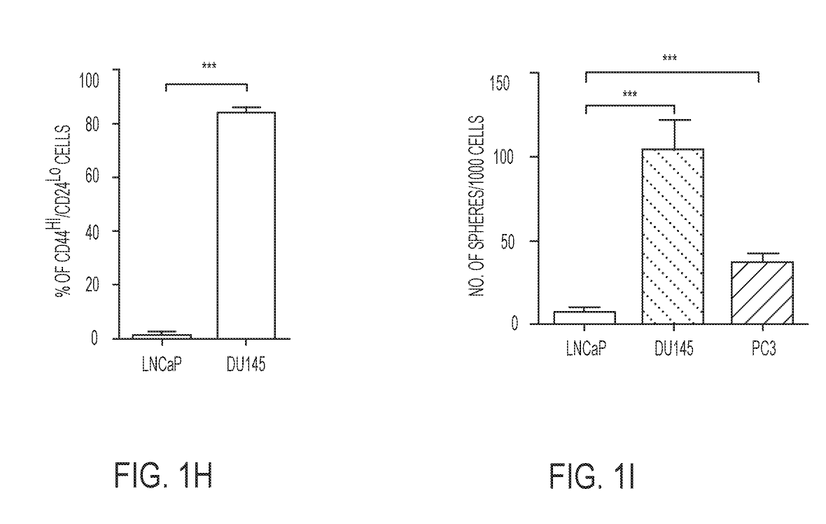

PSA^{-/lo} prostate cancer stem-like cells exhibit augmented EMT properties. It was recently shown that the PSA^{-/lo} subpopulation of cells from primary human prostate tumors, as well as from various PCa cell lines, represent self-renewing, tumor-propagating cells resembling PCaSC (Qin et al., 2012). It was also previously demonstrated that in breast carcinoma, EMT constitutes a major source for the generation of such tumor-propagating stem-like cells (Hollier et al., 2013). In order to investigate the relationship between EMT and PSA, PSA⁺ and PSA^{-/lo} sub-fractions were isolated from the androgen-responsive LNCaP cell line expressing the PSA-promoter driving GFP expression, as described previously in Qin et al., 2012, and the expression of well-characterized EMT markers was analyzed. In comparison to the PSA⁺ (GFP⁺) fraction, the PSA^{-/lo} (GFP^{-/lo}) cells clearly appeared more mesenchymal (FIG. 1A). qRTPCR (FIG. 1B) and western blot (FIG. 1C) analyses revealed that the stem-like PSA^{-/lo} cells exhibited markedly diminished expression of prostate epithelial differentiation (PD) markers AR and PSA, as expected, with simultaneously elevated expression of known stem-cell markers, Bmi1 and Sox2 as well as NE differentiation markers (FIG. 1B). Additionally, PSA^{-/lo} cells also exhibited significantly reduced expression of the epithelial marker E-cadherin, and increased expression of mesenchymal markers and the central EMT regulator, FOXC2 (FIGS. 1B,C). Immunofluorescent staining further confirmed the loss in expression and plasma membrane localization of E-cadherin, with concomitant increase in expression of CSC markers Zeb1 (Wellner et al., 2009; Chaffer et al., 2013) and FOXC2 in PSA^{-/lo} cells (FIG. 1D). These results suggest that even in the androgen-dependent LNCaP cell line, the PSA^{-/low} cells resembling stem-like cells, display the EMT phenotype as well as NE-like features.

Androgen-Independent Metastatic PCa Cell Lines Possess Increased EMT/Stem-Like Features.

While LNCaP cells are androgen-dependent and poorly invasive, the androgen-independent DU145 and PC3 cells are far more invasive and harbor significantly higher metastatic potential (Pulukuri et al., 2005) It was observed that LNCaP cells predominantly exhibited epithelial features including expression of AR/PSA and E-cadherin (FIGS. 1E,F). DU145 and PC3 cells, on the other hand, exhibited significantly increased expression of EMT-associated mesenchymal markers, as well as NE differentiation markers, with simultaneous loss in E-cadherin levels (FIGS. 1E,F). Interestingly, expression of FOXC2 was restricted to the androgen-independent metastatic cell lines-DU145 and PC3, and almost undetectable in the weakly invasive non-metastatic LNCaP cells (FIG. 1F).

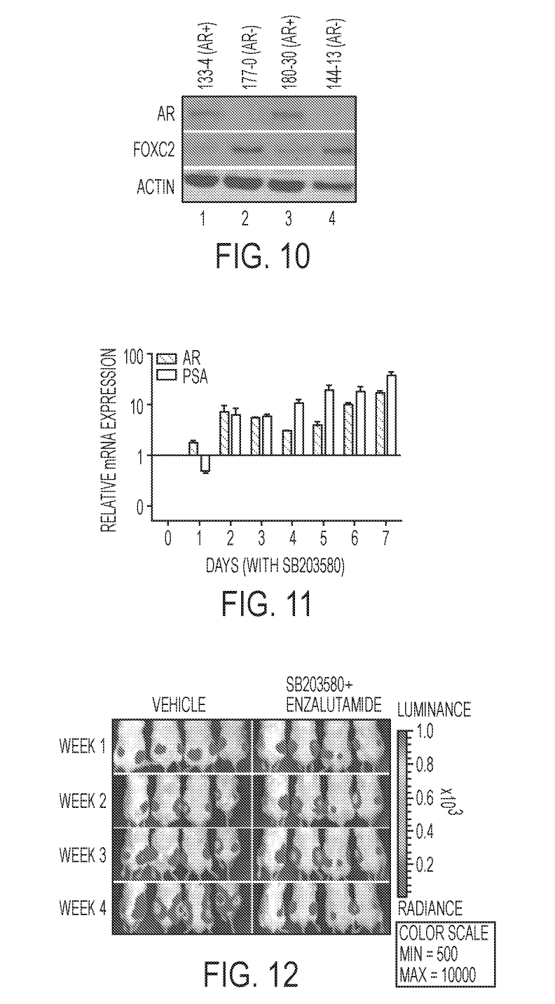

Since FOXC2 is known to empower tumor cells with stem-cell attributes in breast carcinoma, these PCa cell lines were tested for stem-like properties including their ability to form prostospheres, and expression of stem cell-related cell-surface markers CD44 and CD24 (Hurt et al., 2008). In correlation with their reduced sphere-forming capacity in non-adherent cultures, the poorly invasive LNCaP cells harbored less than 1% CD44^{hi}/CD24^{lo} stem-cell enriched fraction (FIGS. 1G,H). In contrast, more than 85% of the metastatic DU145 cells were CD44^{hi}/CD24^{lo} (FIGS. 1G,H), consistent with increased expression of other known stem-cell markers, Bmi1 and Sox2 (FIG. 1E). It was also observed that the two PCa cell lines expressing high levels of endogenous FOXC2-DU145 and PC3-formed significantly higher number of tumorspheres (FIG. 11), indicating higher stem-like potential compared to LNCaP cells, which have very low FOXC2 expression. Interestingly, the PCa cells that have higher metastatic potential and augmented stem-cell properties consistently lack AR/PSA expression (FIGS. 1E,F).

FOXC2 Expression Heralds the Androgen-Independent State Associated with Loss in AR/PSA Expression, Poor Gleason Scoring and Recurrent PCa.

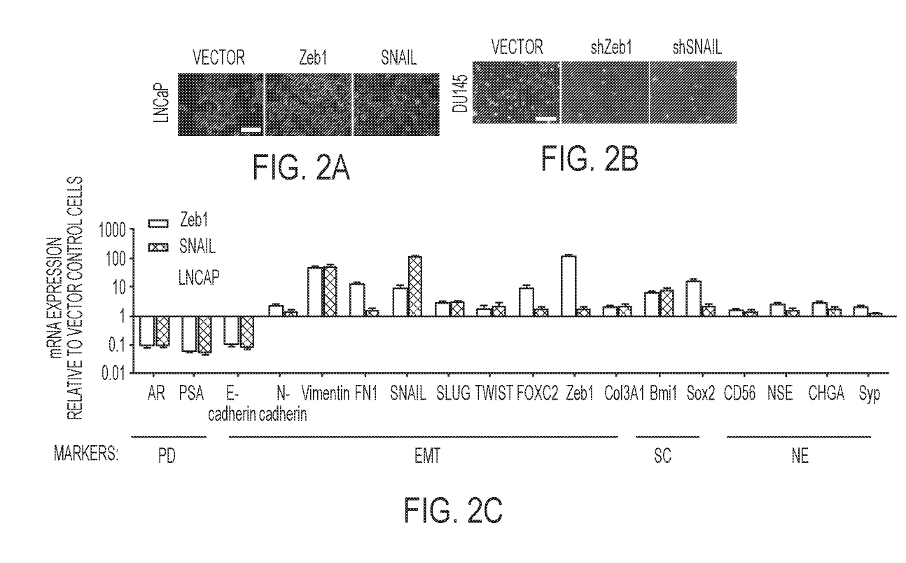

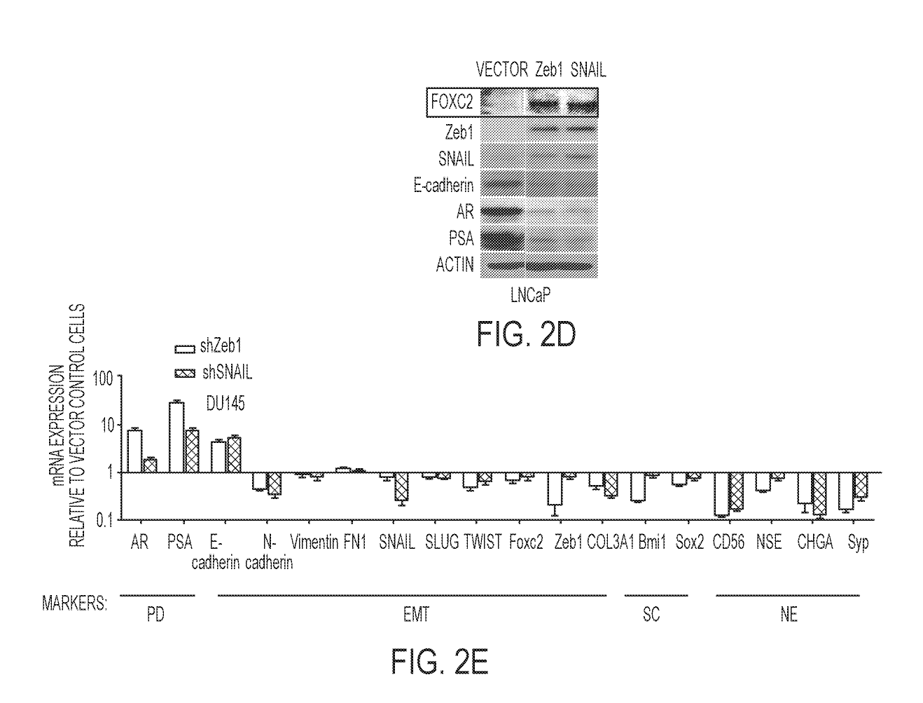

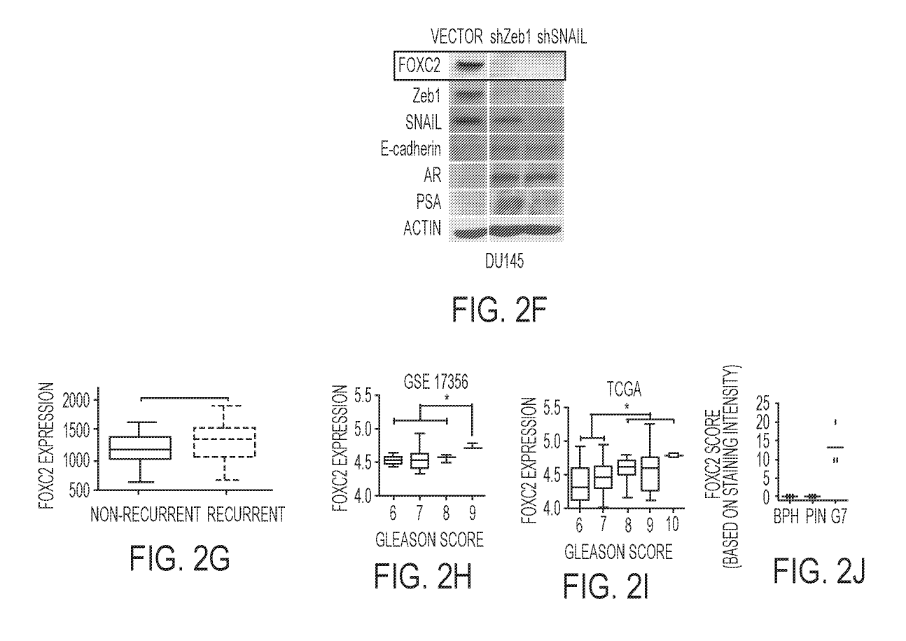

It was previously discovered that FOXC2 is not expressed in differentiated breast cancer cells but is markedly upregulated following EMT, and is enriched in CSC fractions (Hollier et al., 2009). It was therefore examined whether induction of EMT in PCa cells would similarly result in upregulation of FOXC2. Indeed, it was found that overexpression of Snail or Zeb1 results in induction of EMT and NE trans-differentiation, and most importantly, a significant upregulation of FOXC2, with concomitant loss in AR/PSA expression in androgen-dependent LNCaP cells (FIGS. 2A,C,D). Conversely, inhibition of EMT-inducing genes such as Snail, or Zeb1 in highly metastatic DU145 cells, results in a striking loss in FOXC2 expression or NE-like features, with restoration of AR/PSA expression (FIGS. 2B,E,F). Collectively, these findings suggest that FOXC2 possibly functions downstream of various EMT pathways in PCa cells.

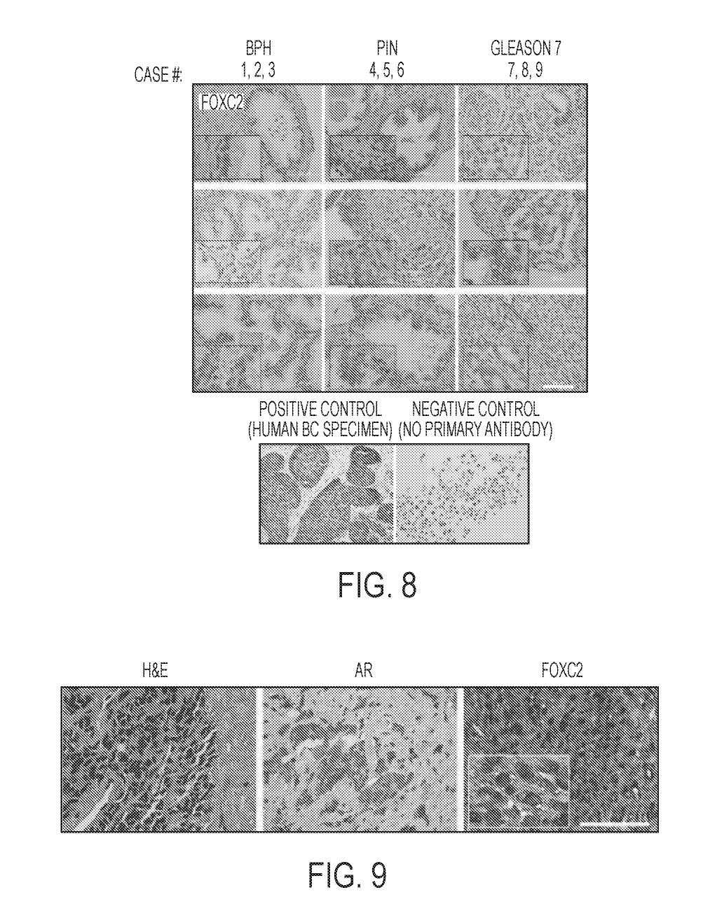

To further investigate the physiological significance of FOXC2 in prostate tumor progression, the GDS4109 GEO database that contains non-recurrent and recurrent PCa samples was analyzed. Notably, FOXC2 expression was significantly higher in the recurrent samples (FIG. 2G). Moreover, a direct correlation was found between markedly increased FOXC2 levels and high Gleason grading, in 2 independent publicly available datasets [GSE17356 (FIG. 2H) and TCGA (FIG. 2I)]. To confirm the relevance of these observations, IHC analyses was performed for FOXC2 protein in patient-derived primary prostate tissue samples ranging from benign prostatic hyperplasia (BPH), to prostatic intraepithelial neoplasia (PIN), to advanced grade Gleason 7. Interestingly, a correlation was discovered between high expression of FOXC2, and high Gleason grading associated poor clinical outcome (FIG. 2J, FIG. 8), reaffirming the earlier observations. Further, in aggressive small-cell carcinoma of the human prostate characterized by lack of AR, significantly elevated expression of FOXC2 was observed (FIG. 9). Similarly, in patient-derived xenograft (PDX) models of human prostate tumors exemplified by castration-resistance, neuroendocrine features and loss of AR (Tzelepi et al., 2012; Aparicio et al., 2011), increased FOXC2 expression was consistently observed (FIG. 10). Together, these data suggest that expression of FOXC2 in PCa cells segregates with the androgen-independent state that is associated with increased stemness.

Enforced Expression of FOXC2 Induces the EMT/CSC Phenotype and Increased Drug-Resistance.

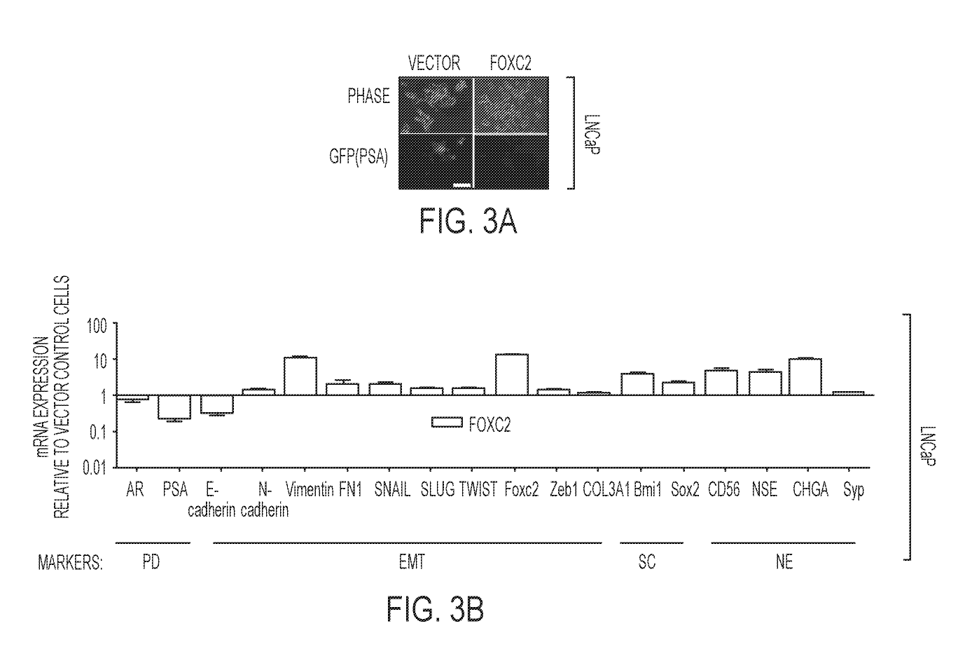

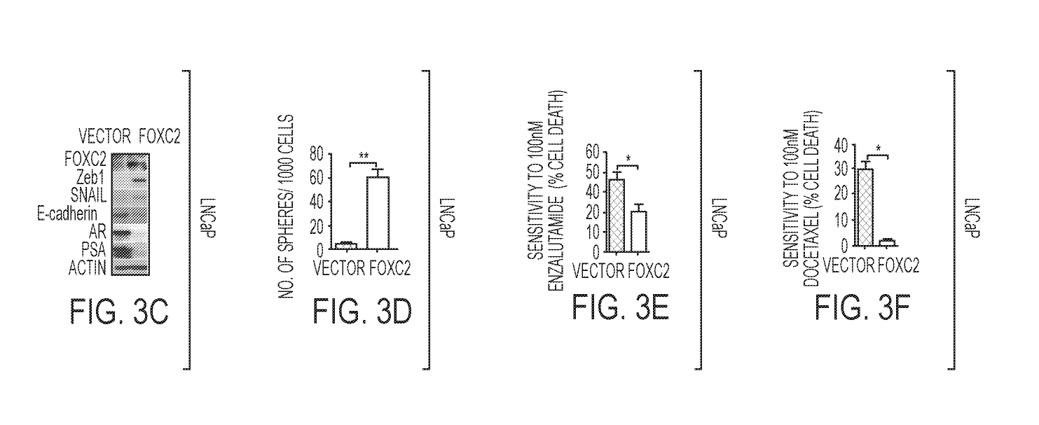

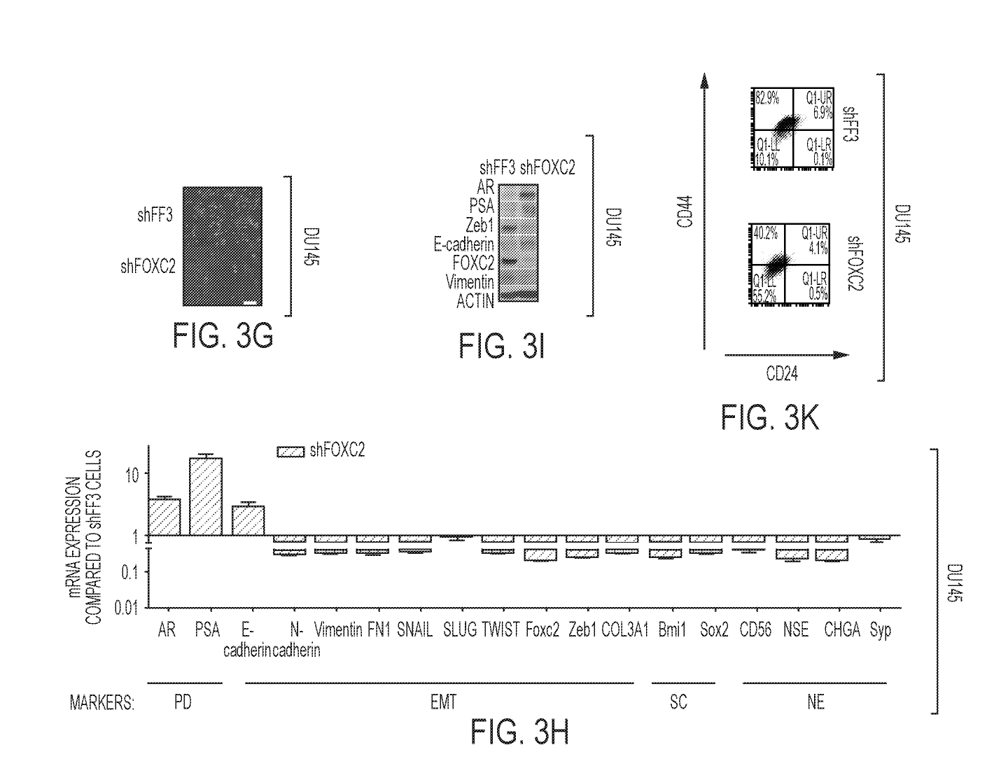

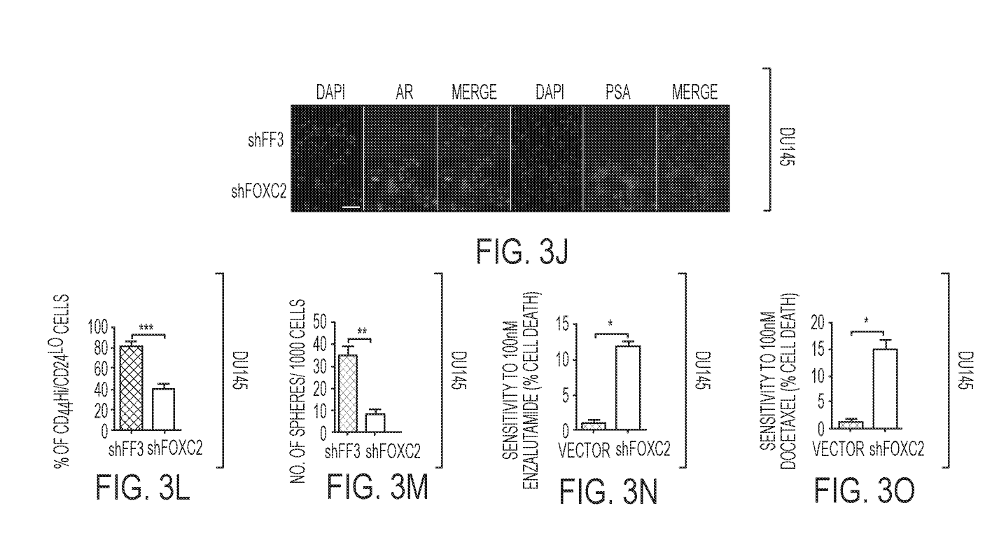

Since FOXC2 is induced downstream of several different EMT inducers, and is by itself capable of potentiating the effects of multiple independent EMT signals (Taube et al., 2010), it was queried how ectopic expression of FOXC2 would impact the behavior of epithelial-like LNCaP cells, which are androgen-dependent. In fact, over-expression of FOXC2 resulted in the generation of cells that displayed the classic mesenchymal phenotype (FIG. 3A-upper panel) and induced the expression of EMT markers (FIGS. 3B,C), along with significant upregulation of known stem-cell markers, Bmi1 and Sox2 (FIG. 3B), as well as common clinical NE markers (FIG. 3B). This was also accompanied by a significant increase in tumor sphere formation (FIG. 3D), suggesting enhanced stem-like function. Again, FOXC2-induced EMT was associated with a reduction in PSA-promoter activity (as determined by loss in GFP) (FIG. 3A-lower panel), as well as loss in AR expression (FIGS. 3B,C). It was also observed that FOXC2 expression rendered androgen-dependent LNCaP cells increasingly resistant to Enzalutamide (FIG. 3E) (a common anti-androgen), and Docetaxel (FIG. 3F) (a common chemotherapeutic used in PCa), using the MTS cell survival assay.

Inhibition of FOXC2 Reduces Stem-Like Properties, and Restores AR/PSA Expression as Well as Drug Sensitivity.

To confirm the contribution of FOXC2-mediated EMT in the generation and maintenance of stem-cell attributes in PCa cells, FOXC2 expression was stable knocked-down in androgen-independent DU145 cells, that have were shown (FIG. 1) to contain a significantly high stem cell-enriched fraction. Loss of FOXC2 expression resulted in the acquisition of a uniform epithelial phenotype in DU145 cells, which are otherwise known to possess a heterogeneous morphology (FIG. 3G), as well as reversal of EMT and NE-like features (FIGS. 3H,I). Interestingly, a significant up-regulation in AR/PSA levels was observed when FOXC2 expression was lost (FIGS. 3H-J). This was accompanied by a massive reduction in self-renewal potential and stem-cell properties in DU145 cells (FIGS. 3K,L,M), including expression of Bmi1 and Sox2 (FIG. 3H). DU145 cells are androgen-independent and insensitive to the AR inhibitor Enzalutamide, even at 10 μM 27. However, suppression of FOXC2 in these cells rendered them more sensitive to Enzalutamide even at 100 nM, as determined by the MTS cell survival assay (FIG. 3N).

Emergence of Docetaxel-resistance in PCa remains an important therapeutic hurdle, and DU145 cells represent a good model system to study mechanisms altering Docetaxel-resistance (Tamaki et al., 2014). Interestingly, loss in FOXC2 expression rendered DU145 cells more susceptible to 100 nM Docetaxel treatment (FIG. 3O). Together, these results suggest that FOXC2 expression is necessary and sufficient for the induction of PCaSC attributes associated with loss in AR/PSA expression and emergence of androgen-independence and chemo-resistance.

FOXC2 Regulates AR Via Zeb1, a Known Transcriptional Repressor.

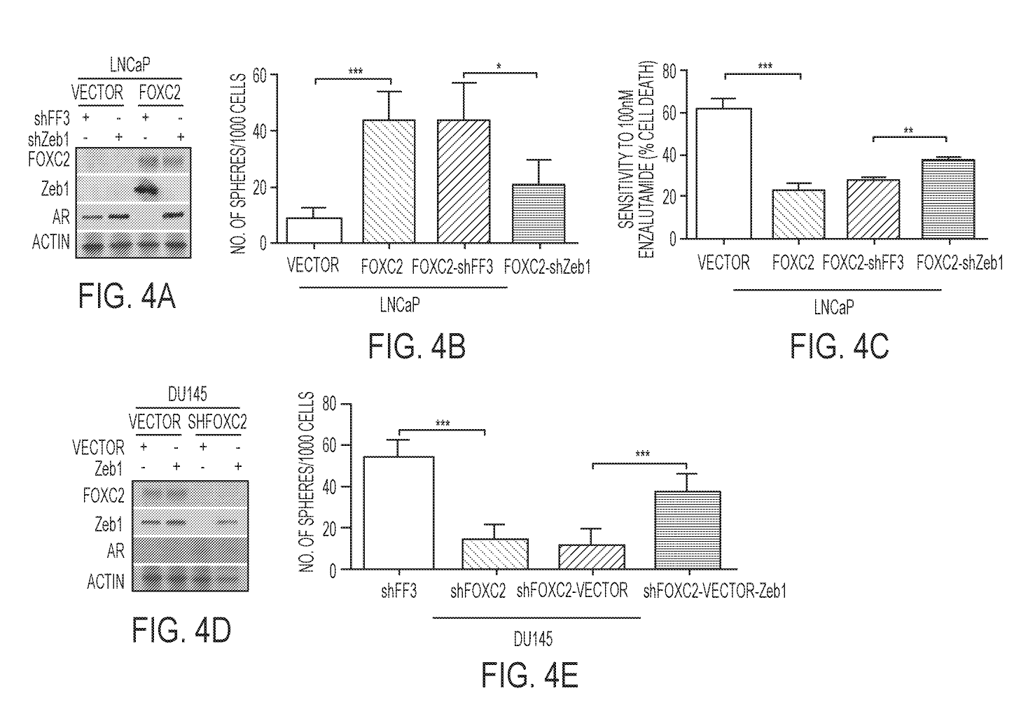

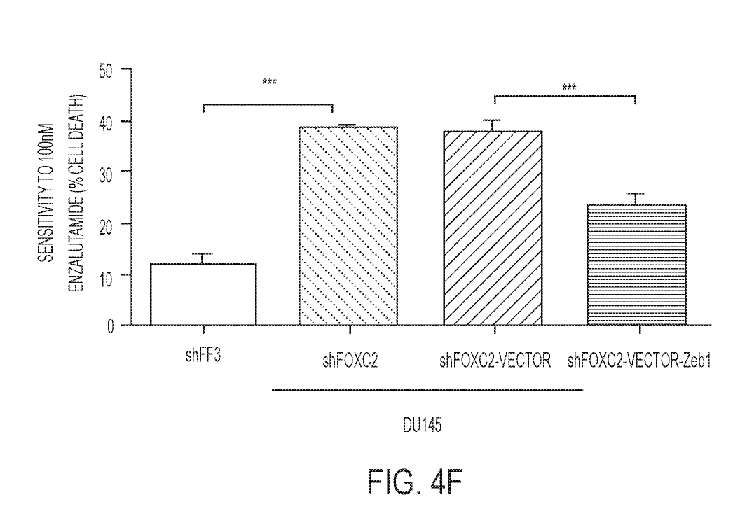

It is notable that although FOXC2 functions as a transcriptional activator in most cell types studied, its expression in PCa cells however, causes a drastic loss in AR/PSA levels. In this context, it is pertinent to reveal parallel studies from our laboratory performed in breast cancer cells, which demonstrate that FOXC2 exerts its repressive effects indirectly through Zeb1, a known transcriptional repressor. Therefore, it was investigated if similar links are operative in PCa cells. In LNCaP cells over-expressing FOXC2, it was observed that enforced loss of Zeb1 drastically obliterates the AR-repressive effect of FOXC2 (FIG. 4A), as well as their stem-like properties (FIG. 4B), and resistance to Enzalutamide (FIG. 4C). Further, over-expression of Zeb1 in DU145 cells lacking FOXC2 expression was sufficient to cause a significant down-regulation in AR levels (FIG. 4D), with restoration of sphere-forming capacity (FIG. 4E) and resistance to Enzalutamide (FIG. 4F). These data suggest that FOXC2-dictated PCaSC attributes associated with loss in AR expression and emergence of ADT-resistance are mediated by Zeb1.

Activation of p38MAPK Signaling Correlates with FOXC2-Associated EMT/Stem-Like Features.

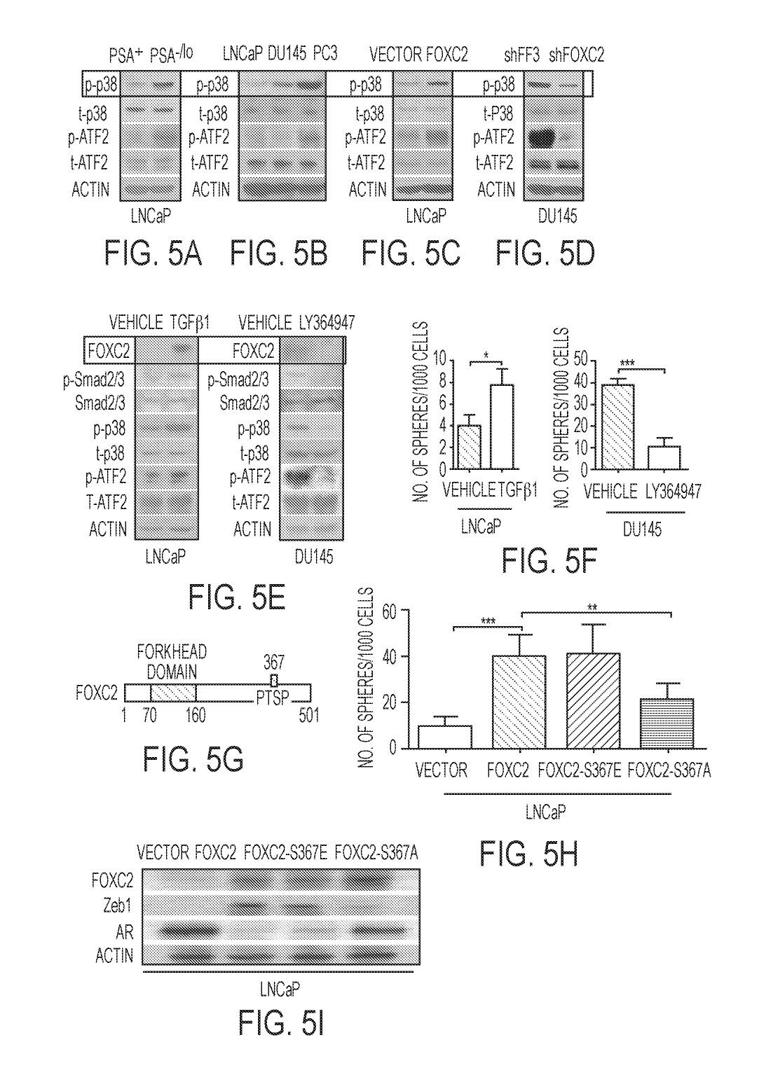

p38 Mitogen-Activated Protein Kinases (MAPK), are known to play key roles in cellular proliferation, differentiation, apoptosis, invasion, and migratio—attributes, that are all significantly altered during the course of cancer progression (Koul et al., 2013). In a parallel study using breast cancer cells, it was found that FOXC2 not only possesses functional phosphorylation sites for p38-MAPK14, but that both FOXC2 and the active form of p38 (phospho-p38) are consistently high in cells that have undergone EMT, as well as in CSC-enriched cell populations. In fact, it was observed that PCa cells with inherent mesenchymal and stem-cell properties (DU145/PC3 relative to LNCaP, or PSA^{hi} relative to PSA^{-lo}) exhibit significantly increased p-p38 and its direct target, Activating Transcription Factor-2 (pATF2) (FIGS. 5A-D), which strongly correlates with FOXC2 expression (FIGS. 1B-F, FIGS. 5C,D).

This correlation between FOXC2 expression and p38 activation was further tested in yet another model of prostate EMT induction, using Transforming-Growth-Factor-β1 (TGFβ). TGFβ1 is a well characterized activator of p38 signaling, and has been shown to induce EMT in a variety of epithelial cell types including the prostate (Shiota et al, 2012). Treatment of LNCaP cells with TGFβ1 resulted in moderate induction of EMT, FOXC2 expression, as well as concurrent activation of p38 signaling (FIG. 5E), and increased stem-like function (FIG. 5F). On the contrary, suppression of TGFβ1 signaling in DU145 cells using LY364947, resulted in concomitant loss in FOXC2 expression and p38 signaling (FIG. 5E), with markedly reduced tumorsphere-forming capacity (FIG. 5F). Together, all the above data suggest that targeting FOXC2, by interfering with p38 signaling, may provide a therapeutic solution to preventing CSC generation/function in androgen-insensitive PCa cells.

Interestingly, a potential p38 phosphorylation site harbored within a consensus sequence was discovered on human FOXC2 protein (FIG. 5G). Next, the functional relevance of this putative site in facilitating FOXC2-mediated stem-cell functions was investigated in PCa cells. While expression of the S367E-FOXC2 mutant, which mimics constitutive p38 phosphorylation, enhanced sphere formation and Zeb1 expression associated with loss in AR, the S367A-FOXC2 mutant, which represents the non-p38-phophorylatable form of FOXC2, resulted in a loss in stem-cell function and Zeb1 expression, with concomitant restoration in AR expression (FIGS. 5H,I). These results suggest that FOXC2 may be a direct target of p38 in PCa cells, and further, that the S367 site plays a key role in mediating FOXC2 functions in these cells.

Suppression of p38 Signaling Results in Reversal of EMT and Significant Decrease in Stem-Like Properties.

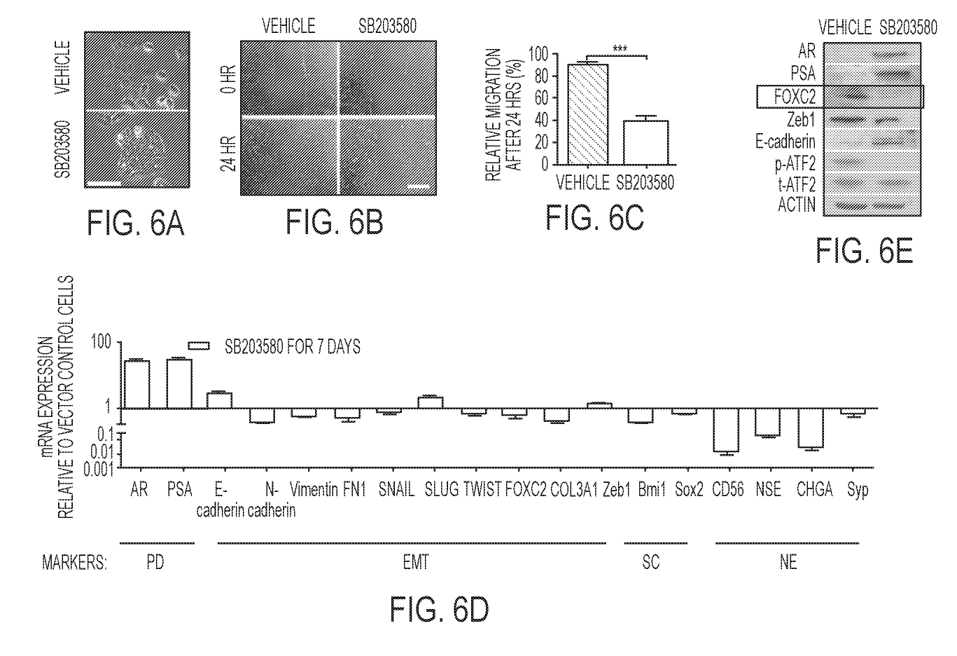

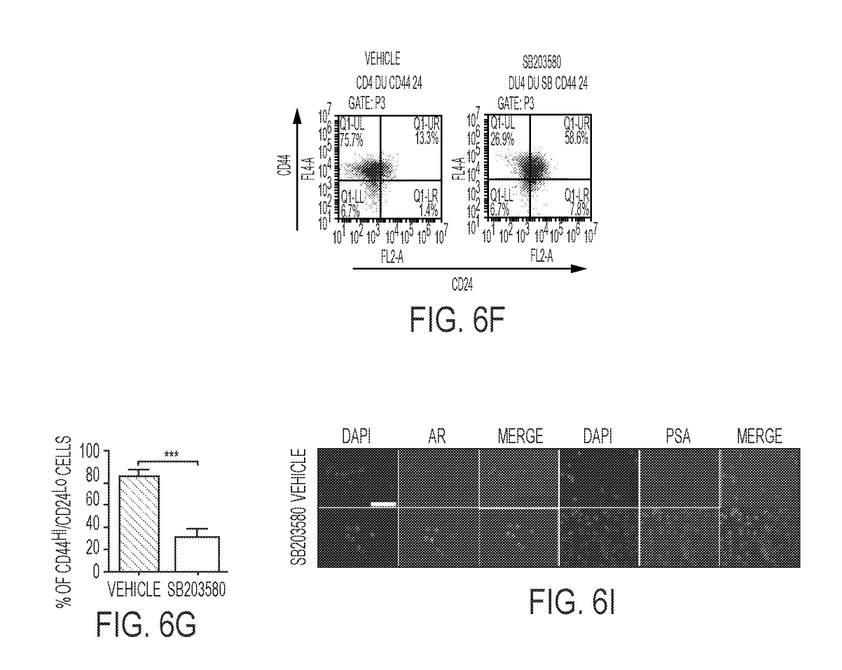

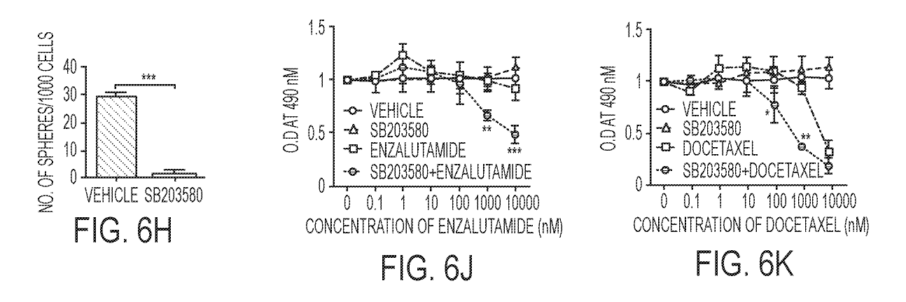

Treatment of DU145 cells with SB203580, a specific chemical inhibitor of p38 signaling, for 7 days, resulted in their acquisition of an epithelial phenotype (FIG. 6A), with significant reduction in migratory potential (FIGS. 6B,C). This was accompanied by a dramatic loss in FOXC2 expression and consequent EMT/CSC features (FIGS. 6D,E), including significant reduction in the CD44^{hi}/CD24^{lo} fraction (FIGS. 6F,G), expression of Bmi1 and Sox2 (FIG. 6D), and tumorsphere formation (FIG. 6H). Interestingly, inhibition of p38 signaling and FOXC2 expression using SB203580 resulted in restoration of AR and PSA expression in DU145 cells (FIGS. 6D,E,I). qRTPCR performed progressively from days 0-7 after SB203580 treatment revealed that expression of AR, PSA and E-cadherin consistently increased right after 48 hours, but reached highest levels at day 7 (FIG. 11). Interestingly, such restoration of AR/PSA expression with simultaneous loss in FOXC2 brought about by SB203580 pre-treatment, significantly resensitized these cells to subsequent co-treatment with SB203580 and either Enzalutamide (FIG. 6J) or Docetaxel (FIG. 6K), suggesting that a combinatorial approach might be effective in targeting highly invasive PCa cells that are resistant to ADT and standard chemotherapy.

Combinatorial Suppression of p38 Signaling with Enzalutamide Treatment Significantly Reduces Tumor Size.

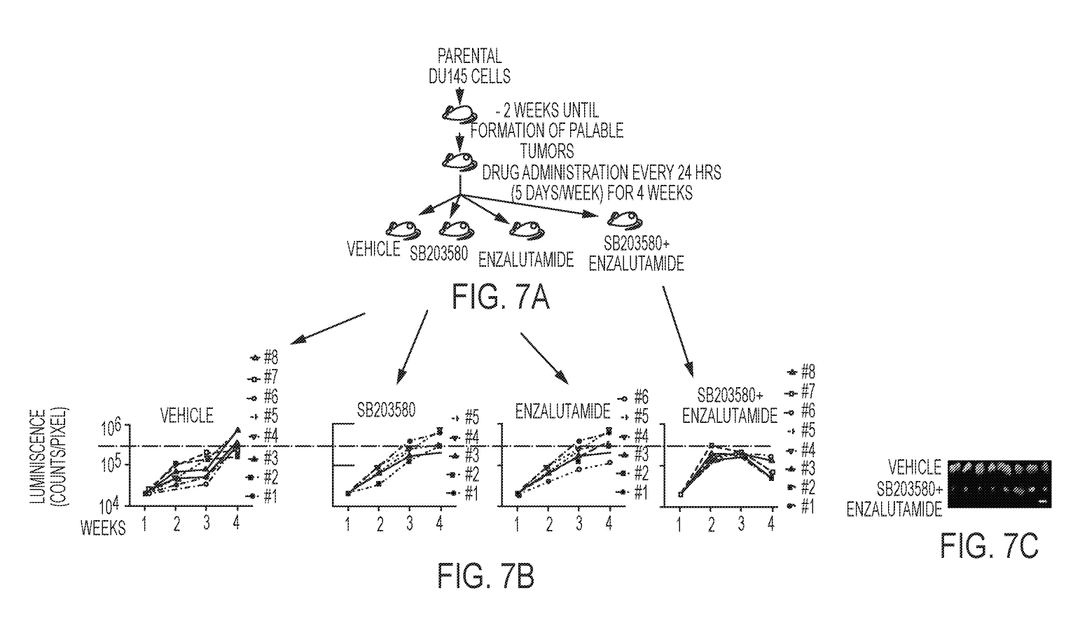

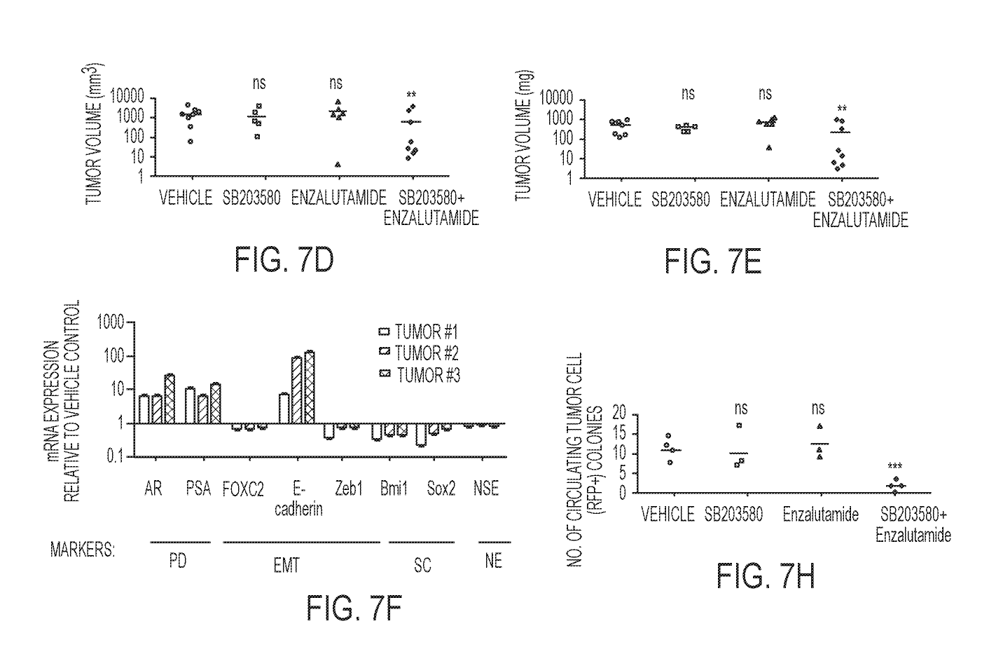

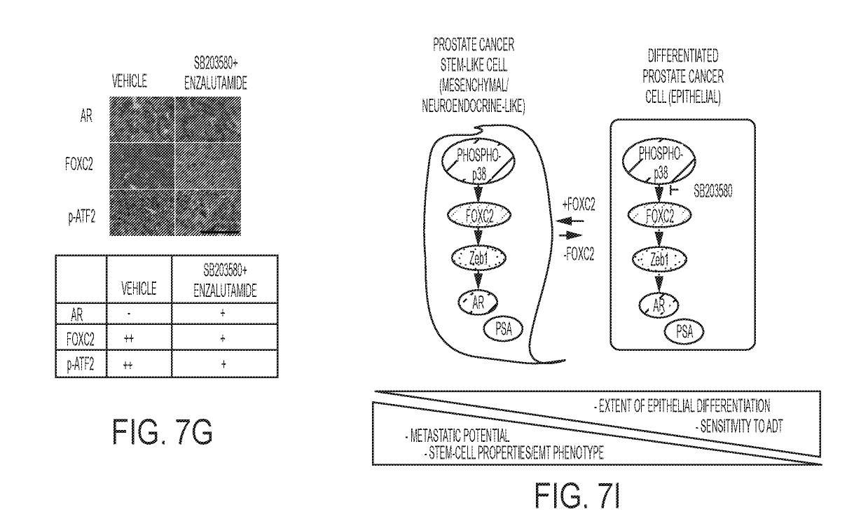

To investigate the effect of inhibition of FOXC2-dependent EMT/CSC attributes on tumor formation in vivo, NOD/SCID mice were subcutaneously injected with DU145 cells and began treatment in vivo, after formation of palpable tumors. The treatment plan/schedule is depicted in FIG. 7A. While there was no appreciable change in tumor size in mice treated singly with either SB203580 or Enzalutamide compared to the vehicle-treated group, mice treated with a combination of SB203580 and Enzalutamide showed a significant decrease in tumor volume and weight (FIGS. 7B-E, FIG. 12). This suggested that although parental DU145 cells are Enzalutamide-resistant to begin with, co-treatment of cells with the p38 inhibitor and Enzalutamide, once again confers susceptibility to ADT. In direct corroboration of the in vitro results (FIG. 6), such a combinatorial treatment in mice bearing aggressive DU145 tumors, reduced FOXC2 expression and consequent EMT/CSC/NE features, and facilitated restoration of AR/PSA expression in the tumors (FIGS. 7F,G).

EMT has long been recognized to provide a “passport” to tumor cells allowing their invasion of tissue boundaries and entry into circulation. These “altered” circulating tumor cells (CTC) then go on to sow the seeds for distant metastasis. In support of an important role for FOXC2-mediated EMT/CSC properties facilitating prostate tumor cell shedding into circulation, we indeed observed that combinatorial treatment markedly reduced the number of CTCs in mice, quantified as RFP-positive CTC colonies isolated from blood (FIG. 7H).

It is important to note that suppression of FOXC2 in invasive AR^{-/lo} DU145 PCa cells was sufficient to restore AR expression and function, as judged by up-regulation of its downstream target, PSA (FIGS. 3,6,7). Restoration of AR/PSA expression and the associated epithelial state with loss of stem-like functions (either using SB203580, or directly, through FOXC2 suppression) rendered these androgen-independent cells once again sensitive to Docetaxel and Enzalutamide, with substantial loss of tumorigenic NE-like features (FIGS. 3,6,7). These observations lend further support to the view that androgens and AR demonstrate crucial tumor suppressive effects in the prostate. It is in this context that the concomitant use of the p38 inhibitor is proposed, which offers the dual benefit of impeding FOXC2-mediated EMT/CSC attributes, while simultaneously restoring AR-derived protection.

In conclusion, it has been demonstrated that FOXC2 is an important determinant of prostate cancer stem-like attributes, dictating the biochemical shift to ADT- and chemo-resistance (FIG. 7I). Accordingly, a novel and tangible method is provided herein to target FOXC2 functions in vivo, at least in part, through systemic inhibition of p38 signaling. Targeting FOXC2 curtails prostate tumor cell plasticity, by preventing both EMT, as well as NE trans-differentiation. Future efforts will be directed to examining the molecular basis of FOXC2 function as it represents a cornerstone to the understanding of the fundamental mechanisms dictating stem-cell function and tumor progression in a significant subpopulation of patients harboring variant forms of PCa (such as NEPC or small-cell prostate carcinomas) that are defined by lack of AR/PSA expression, as well as in metastatic CRPCs that arise following ADT.

### Example 2—Materials and Methods

Cell lines: Authenticated LNCaP, DU145, and PC3 cells were procured from ATCC and cultured in RPMI with 10% fetal bovine serum (FBS) with penicillin/streptomycin. Cells overexpressing EMT transcription factors and shRNA were also cultured in the same media. HEK293T cells were cultured in DMEM with 10% FBS and penicillin/streptomycin. All cell lines used for this study were recently confirmed negative for mycoplasma contamination. TGFβ1, LY364947, and SB203580 were used at a final concentration of 5 ng/ml, 1 μM and 5 μM respectively.

Inhibitor Experiments—Cell Culture:

For assessing the effect of inhibition of p38MAPK signaling, cells were treated for 7 days with 5 μM SB203580 45 (EMD-Millipore) dissolved in water. For assessing the combined effect of p38MAPK inhibition and the AR antagonist Enzalutamide (Scher et al., 2012) (SelleckChem) or the chemotherapeutic Docetaxel (LC Laboratories), cells were co-treated with SB203580 and either Enzalutamide/Docetaxel for 7 days. It is important to note that the concentration of SB203580 used to treat PCa cells in our experiments, results in selective inhibition of p38MAPK signaling, as also suggested in (Davies et al., 2000). No appreciable changes in the phosphorylation of Akt1, another potential target, were observed.

TABLE 1
Target shRNA sequences

| Target | shRNA Sequence (5′ to 3′) |
|---|---|
| FOXC2 (#4) | CCAGTGCAGCATGCGAGCGAT |
| FOXC2 (#5) | AGAACATCATGACCCTGCGAA |
| Zeb1 (625) | TAATTTGTAACGTTATTGC |
| Zeb1 (184) | TATTCTCTATCTTTTGCCG |
| Snai1 (1) | ACTTCTTGACATCTGAGTG |
| Snai1 (2) | TGTGGAGCAGGGACATTCG |

TABLE 2
Antibodies

| Primary Antibody | Source | Catalog # |
|---|---|---|
| β-actin | Cell Signaling Technology | 4970L |
| AR | Santacruz | 7305 |
| E-cadherin | BD Biosciences | 610181 |
| FN1 | BD Biosciences | 610077 |
| FOXC2 | Dr. Nayoyuki Miura | N/A |
| N-cadherin | BD Biosciences | 610920 |
| p38 | Cell Signaling Technology | 9212S |
| p-ATF2 | Cell Signaling Technology | 9221S |
| p-p38 | Cell Signaling Technology | 4511S |
| PSA | Santacruz | 7638 |
| Snail | Santacruz | 28199 |
| Total-ATF2 | Cell Signaling Technology | 9226S |
| Vimentin | Novus | H00007431-M01 |
| Zeb1 | Santacruz | 25388 |

TABLE 3
Primer Sequences

| Target (F/R) | Primer Sequence (5′ to 3′) |
|---|---|
| AR-F | 5′-GACGCTTCTACCAGCTCACC-3′ |
| AR-R | 5′-GCTTCACTGGGTGTGGAAAT-3′ |
| Bmi1-F | 5′-CCAGGGCTTTTCAAAAATGA-3′ |
| Bmi1-R | 5′-CCGATCCAATCTGTTCTGGT-3′ |
| CDH1-F | 5′-TGCCCAGAAAATGAAAAAGG-3′ |
| CDH1-R | 5′-GTGTATGTGGCAATGCGTTC-3′ |
| CDH2-F | 5′-GACAATGCCCCTCAAGTGTT-3′ |
| CDH2-R | 5′-CCATTAAGCCGAGTGATGGT-3′ |
| Col3A1-F | 5′-GGGAACAACTTGATGGTGCT-3′ |
| Col3A1-R | 5′-CCTCCTTCAACAGCTTCCTG-3′ |
| FN1-F | 5′-CAGTGGGAGACCTCGAGAAG-3′ |
| FN1-R | 5′-GTCCCTCGGAACATCAGAAA-3′ |
| FOXC2-F | 5′-AGAATTACTACCGGGCTGCG-3′ |
| FOXC2-R | 5′-TGAGCGCGATGTAGCTGTAG-3′ |
| HPRT-F | 5′-TGCTCGAGATGTGATGAAGG-3′ |
| HPRT-R | 5′-TCCCCTGTTGACTGGTCATT-3′ |
| PSA-F | 5′-AGTGCGAGAAGCATTCCCAA-3′ |
| PSA-R | 5′-GAAGCTGTGGCTGACCTGAA-3′ |
| Slug-F | 5′-GAGCATTTGCAGACAGGTCA-3′ |
| Slug-R | 5′-GCTTCGGAGTGAAGAAATGC-3′ |
| Snai1-F | 5′-ACCCCACATCCTTCTCACTG-3′ |
| Snai1-R | 5′-TACAAAAACCCACGCAGACA-3′ |
| Twist1-F | 5′-GGAGTCCGCAGTCTTACGAG-3′ |
| Twist1-R | 5′-CCAGCTTGAGGGTCTGAATC-3′ |
| Vimentin-F | 5′-GAGAACTTTGCCGTTGAAGC-3′ |
| Vimentin-R | 5′-TCCAGCAGCTTCCTGTAGGT-3′ |
| Zeb1-F | 5′-GCACAACCAAGTGCAGAAGA-3′ |
| Zeb1-R | 5′-CATTTGCAGATTGAGGCTGA-3′ |
| CHGA-F | 5′-TGAAATGCATCGTTGAGGTC-3′ |
| CHGA-R | 5′-ACCGCTGTGTTTCTTCTGCT-3′ |
| SYP-F | 5′-TAGGACCCAAGGTGGTCTTG-3′ |
| SYP-R | 5′-TACTCTGGAGCCCACCATTC-3′ |
| CD56-F | 5′-CGGCATTTACAAGTGTGTGG-3′ |
| CD56-R | 5′-GACATCTCGGCCTTTGTGTT-3′ |
| NSE-F | 5′-GTCCCACGTGTCTTCCACTT-3′ |
| NSE-R | 5′-CCCAAGTCAGGCCAGTTTTA-3′ |

Vectors:

The use of the pCS-PSAP-EGFP-DsRed vector has been described previously in Qin et al., 2012. pQXIP-Zeb1 was provided by Dr. Harikrishna Nakshatri (Indiana University, Indianapolis), and pBabePuro-Snail, pBabePuro-FOXC2 and pMIG-FOXC2 by Dr. Robert Weinberg (Whitehead Institute, MIT). S367E-FOXC2 and S367A-FOXC2 mutant constructs were generated by site-directed mutagenesis and subcloned into the retroviral vector MSCV-IRES-GFP. The pLKO1 lentiviral vectors with shFOXC2 & shSnail, and pGIPZ lentiviral vector with shZeb1 were procured from MD Anderson shRNA Core Facility. Two independent shRNA sequences targeting different regions of FOXC2 5′ UTR were used for FOXC2 knockdown, with similar results (data shown is representative from one of them). Similarly, for shSnail and shZeb1 as well. shRNA targeting firefly luciferase (shFF3) was used as a control. Sequence details are in Table 1.

Isolation of Circulating Tumor Cells:

Immediately after sacrificing the mice, ˜100 μl blood was isolated via venipuncture in EDTA-treated collection tubes and stored on ice. Within 30 minutes, the blood was spun down at 1200 rpm for 5 minutes, and the pellet resuspended in 1 ml ACK-lysing buffer (Life Tech) and further incubated for 3-5 minutes. Cells were washed once with PBS, resuspended in RPMI with 10% FBS and pen/strep, and cultured on 10 cm tissue culture dishes. RFP-positive colonies (originating from the labeled DU145 cells injected into mice) were counted after 3-4 days in culture and quantified.

Immunoblotting, Immunofluorescence, RTPCR, wound healing- and tumorsphere assays were performed as previously described (Sarkar et al., 2015). Antibody- and primer details are provided in Tables 2 and 3, respectively. Cell viability (MTS) was assessed using the CellTiter96 Aqueous One-Solution Cell Proliferation Assay kit (Promega).

Immunohistochemistry:

Human prostate tumor tissue samples representing BPH, PIN, advanced Gleason Grade 7, and NEPC were obtained from Drs. Nupam and Kiran Mahajan. Pathological evaluation was performed by two independent pathologists who agreed on the IHC scoring and the positive pattern expression of the markers. The IHC scoring per se was performed in a “blinded” fashion (wherein the scorers did not have access to disease classification/group allocation information) using the H-score system, including intensity (from 0 to 3+) and percentage of positive cells (from 0 to 100%), with a final scoring ranging from 0 to 300.

Flow Cytometry:

Fluorescence-activated cell sorting (FACS) for PSAHi & PSA−/lo cells and CD44Hi & CD24Lo cells was performed as described previously (Hollier et al., 2013) using BD Influx sorter.

Animal Experiments:

˜4 week-old male NOD.CB17-Prkdcscid/J mice were purchased from the Jackson Laboratory (Maine, USA). All animal procedures were verified and approved by the Institutional Animal care and Use Committee of UTMDACC. To study primary prostate tumor formation, 2×106 RFP-luciferase-labelled DU145 cells were injected subcutaneously on both the flanks of 6 week-old male NOD/SCID mice. Once palpable tumors were discernable, tumor-bearing mice were randomly segregated into 4 groups, and drug treatment was initiated every 24 hours for 5 days/week. SB203580 (0.2 μmols in 100 μl per ˜20 g mouse), Enzalutamide (10 mg/kg), or a combination of both drugs (or vehicle) were administered subcutaneously, and tumor growth was assessed as described previously (Hollier et al., 2013). Investigators were blinded to the group allocation while assessing experimental outcomes. At the end of the treatment period, tumors were excised, average diameter calculated using calipers, and tumor weight noted. Tumors were then processed for RNA isolation and/or fixed in formalin, paraffin-embedded, sectioned and stained with hematoxylin/eosin and pATF2-, AR- and FOXC2 antibodies.

Statistical Analyses:

Unless otherwise stated, all samples were assayed in triplicate. All in vitro experiments were repeated at least 3 independent times, and all animal experiments included at least 5 mice per group in each study. Unless otherwise indicated, data are represented as Mean±SEM, and significance calculated using Student's unpaired 2-tailed t-test.

### Example 3—Phosphorylation of Serine 367 of FOXC2 by p38 Regulates ZEB1 and Breast Cancer Metastasis, without Impacting Primary Tumor Growth

FOXC2 Expression Correlates with p38 Activation in Cells Displaying Mesenchymal and Stem Cell Traits.

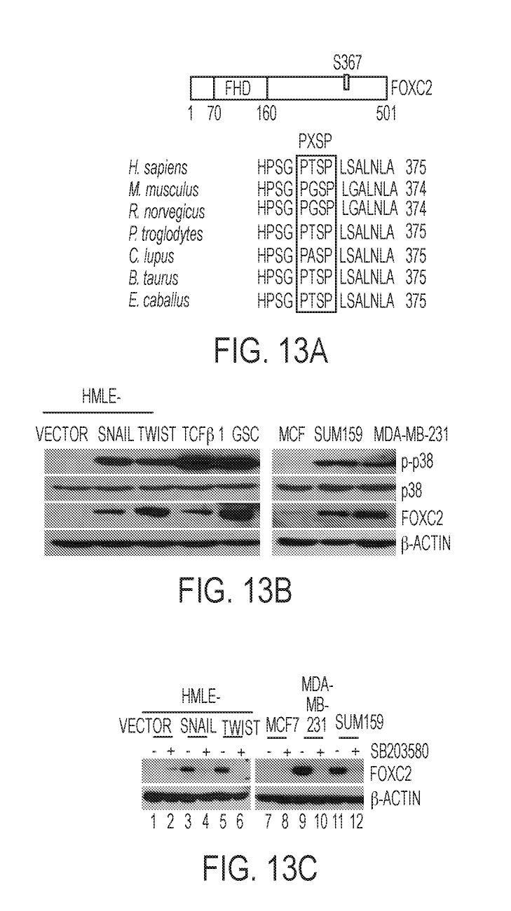

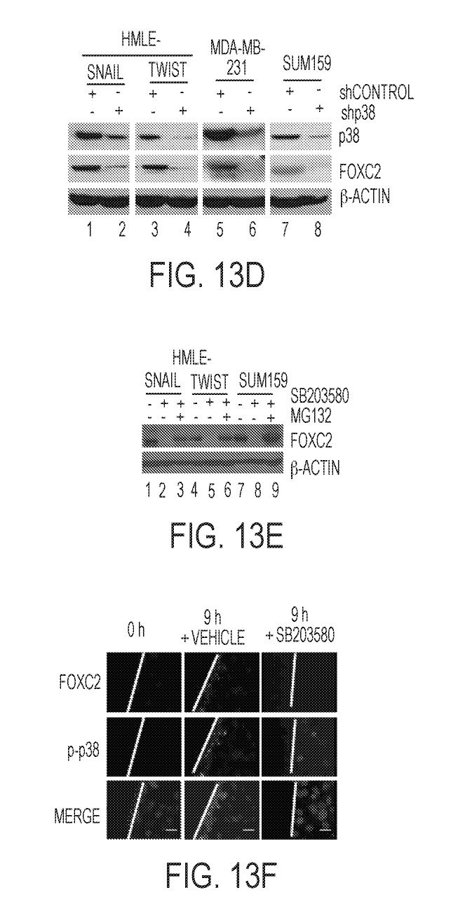

To identify kinases that might regulate FOXC2 function, its aminoacid sequence was analyzed for putative phosphorylation sites using Scansite, an online search engine that identifies short protein sequence motifs likely to be phosphorylated by known serine/threonine and tyrosine kinases (Obbenauer et al., 2003). Under high stringency conditions, we identified an evolutionarily well-conserved consensus phosphorylation motif for p38 associated with serine 367 (S367) of FOXC2 (FIG. 13A).

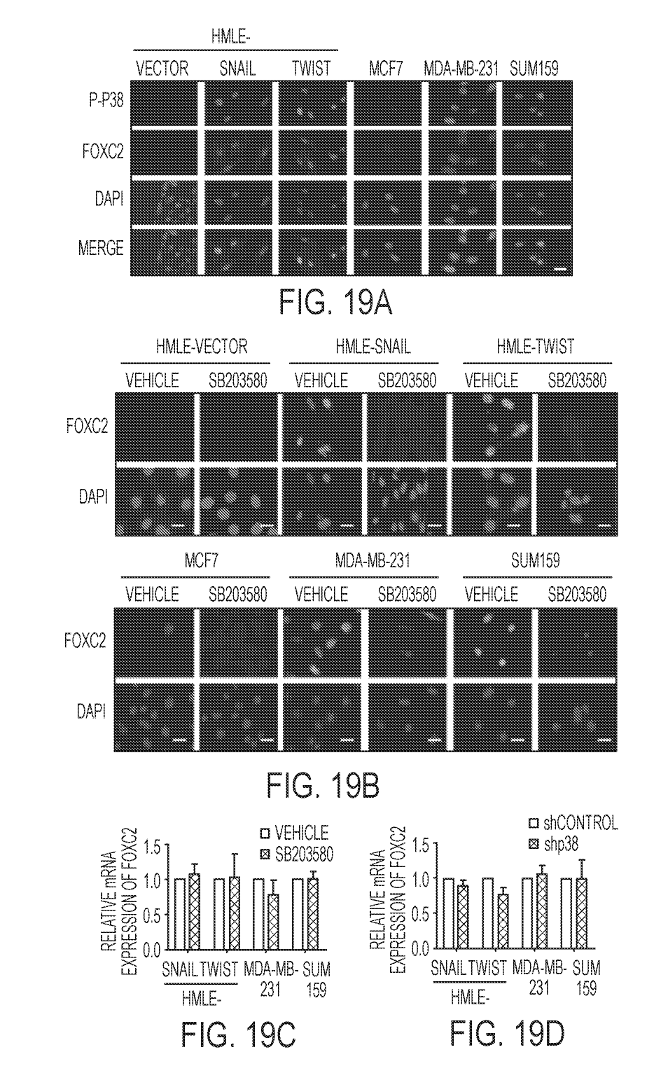

Since FOXC2 expression is restricted to cells with stem cell properties, it was reasoned that if p38 were a major upstream positive regulator of FOXC2 function, the active/phosphorylated form of p38, phospho-p38 (p-p38), would be present only in cells that express FOXC2. Therefore, immortalized human mammary epithelial (HMLE) cells (Elenbaas et al., 2001), experimentally induced to undergo EMT via ectopic expression of Snail, Twist, TGFβ1 or Goosecoid (GSC), and two CSC-enriched human breast cancer cell lines (SUM159, MDA-MB-231), known to express high levels of endogenous FOXC2 were analyzed. Using immunoblotting (FIG. 13B) and immunofluorescence (FIG. 19A), significantly elevated levels of p-p38 were detected in FOXC2-expressing stem cell-enriched mesenchymal mammary cell lines relative to their more differentiated, epithelial counterparts (HMLE-vector, MCF7) (FIGS. 13B; 19A). Of note, comparable total levels of p38 were found in all cases (FIG. 13B).

To examine the functional relationship between p-p38 and FOXC2, a series of cell lines were treated with the pyridinylimidazole SB203580 that inhibits p38 catalytic activity by binding to the ATP-binding pocket, without preventing p38 phosphorylation by upstream kinases (Kumar et al., 1999). Relative to vehicle-treated controls, SB203580 elicited a consistent and striking decrease in FOXC2 protein levels, suggesting that p38 regulates FOXC2 steady-state levels (FIGS. 13C, 19B). To confirm the involvement of p38, shRNA that decreased p38 levels by 50-70% was used, and it was observed a significant reduction in FOXC2 protein levels compared to control shRNA (FIG. 1D). Importantly, neither SB203580 nor p38 shRNA had a discernible effect on FOXC2 transcript levels (FIGS. 19C, 19D). In addition, the proteasome inhibitor MG132 rescued the proteolytic degradation of FOXC2 following SB203580 treatment (FIG. 13E).

Consistent with the activation of EMT at sites of wounding (Yan et al., 2010), it was found that p-p38 and FOXC2 accumulated in the nuclei of cells at the leading edge of a scratch induced in an epithelial HMLE cell monolayer, 9 h post-wound induction (FIG. 13F). Moreover, SB203580 treatment abrogated the upregulation of FOXC2 at the leading edge (but not of p-p38 since SB203580 does not impact p38 phosphorylation). Collectively, these findings reveal a striking correlation between the presence of p-p38 and FOXC2 expression in cells displaying mesenchymal and stem cell traits, as well as in mammary epithelial cells induced to undergo EMT during wound healing, and suggest that p38 regulates FOXC2 protein levels through a post-translational mechanism.

p38 Phosphorylates FOXC2 at S367.

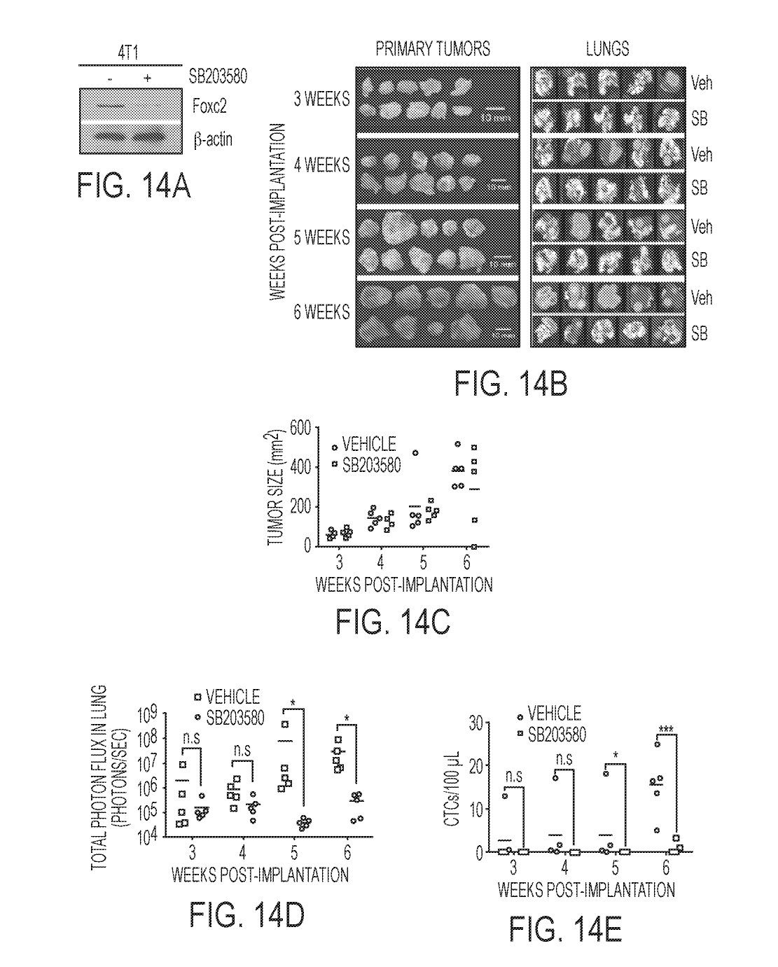

To determine whether p38 and FOXC2 interact with one another, we performed reciprocal co-immunoprecipitation studies in MDA-MB-231 cells and found that endogenous p38 co-immunoprecipitates with FOXC2 and vice versa (FIGS. 14A, 14B). HA-tagged p38 and Myc-tagged FOXC2 were co-expressed in HEK293T cells, immunoprecipitated for either HA or Myc, and analyzed the resulting immunoprecipitates by immunoblotting with Myc- and HA-antibodies respectively (FIGS. 14C and 14D). These data confirmed the direct interaction between FOXC2 and p38, which is contingent on the activation status of p38, since it was abolished when a kinase-dead mutant of p38 (HA-p38-DN) was substituted during transfection (FIG. 14D). This suggests that, among other factors, the interaction of p38 with FOXC2 depends on the configuration of the p38 catalytic cleft. To confirm that FOXC2 serves as a p38 substrate, an in vitro kinase assay was performed (FIG. 14E). p38-dependent phosphorylation of FOXC2 was detected with N-terminally truncated FOXC2, comprising amino acids 245-501, but not with a C-terminally truncated FOXC2, numbering amino acids 1-244, devoid of the putative p38 phosphorylation site. Moreover, FOXC2 phosphorylation was not observed when S367 was mutated to a non-phosphorylatable alanine residue (S367A; FIG. 14E). Collectively, these data suggest that p-p38 physically interacts with FOXC2 and phosphorylates it at S367.

Targeting p38 Selectively Inhibits Metastasis Leaving Primary Tumor Growth Unabated.

Given the role of FOXC2 in bestowing metastatic competence, the above data suggest that disrupting the p38-FOXC2 interaction, using a p38-inhibitor, may perturb tumor progression. In order to investigate this, the 4T1 mouse mammary carcinoma model, which recapitulates many of the characteristics of human breast cancer, was employed. Most notably, 4T1 cells can spontaneously metastasize from a site of orthotopic implantation—the mammary gland—to the lung in syngeneic wild-type immunocompetent mouse hosts (Aslakson et al., 1992; Pulaski et al., 2001).

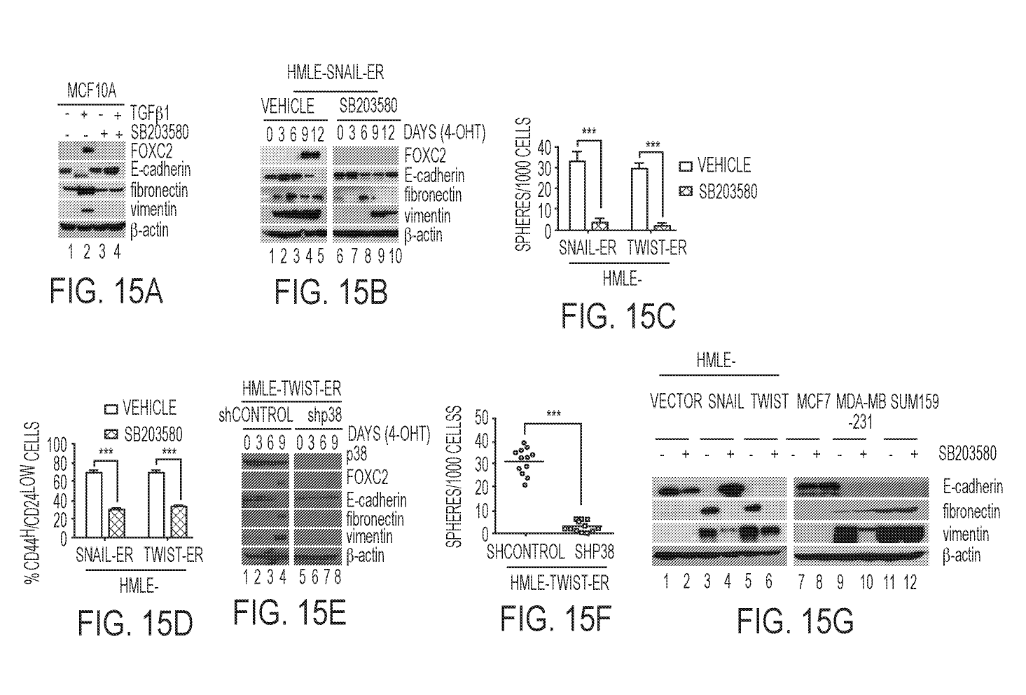

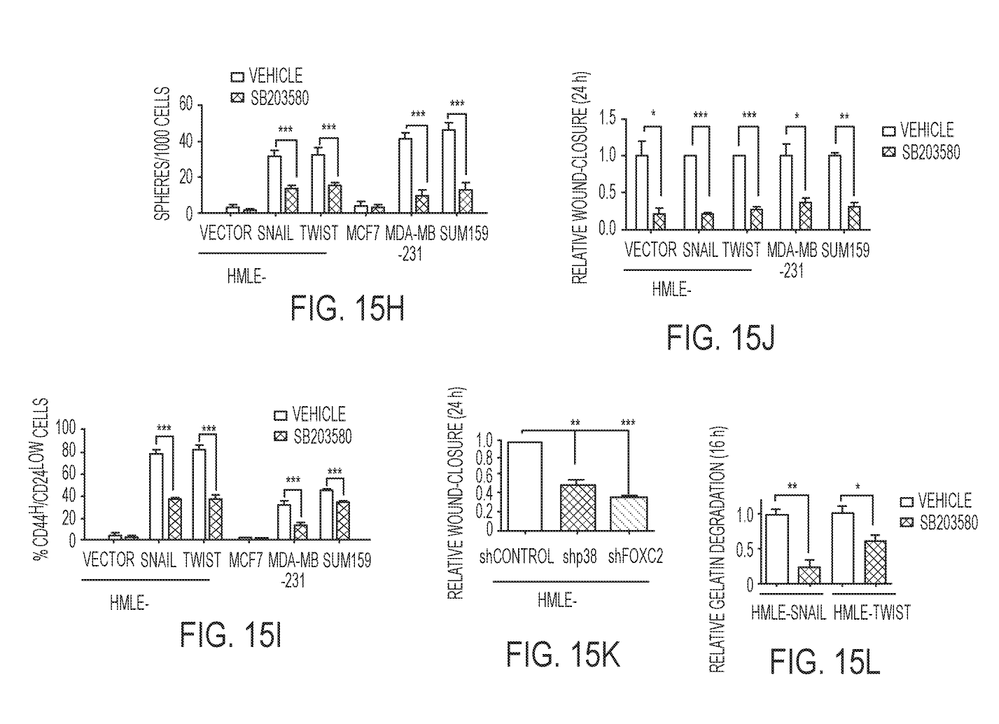

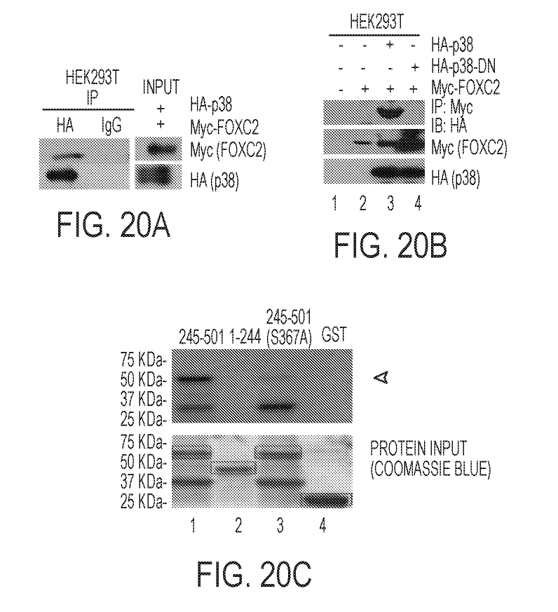

First, a significant reduction of endogenous Foxc2 protein levels was confirmed following treatment of 4T1 cells with SB203580 (FIG. 15A). Next, luciferase-labeled 4T1 cells were orthotopically implanted into the fourth mammary fat-pads of 40 female BALB/c mice and treated them daily with vehicle or SB203580 (20 mice per group). Tumor progression was monitored weekly using caliper measurements and bioluminescence. Starting week 3 post-implantation, 5 mice per group were sacrificed and surgically excised the primary tumors and lungs were surgically excised. Unexpectedly, SB203580-treated mice formed primary tumors of a similar size relative to vehicle-treated counterparts (FIG. 15B, left panels). This observation was corroborated by caliper measurements (FIG. 15C) and the bioluminescent signal emitted by these tumors (FIGS. 20A, 20B). Indeed, there were no significant differences in the latency and growth rates of the vehicle- and SB203580-treated primary tumors during this timecourse (FIGS. 15C and 20B).

Contrary to the observations with primary tumors, mice treated systemically with SB203580 exhibited strikingly fewer lung metastases, as evidenced by the markedly reduced bioluminescent signal relative to vehicle-treated counterparts (FIG. 15B, right panels and 15D). Consistent with these observations, macroscopic and histological examination revealed the presence of multiple nodules in the lungs of vehicle-treated mice compared to much fewer nodules in the lungs of SB203580-treated counterparts (FIGS. 20C, 20D). Collectively, these findings suggest that p38 inhibition selectively prevents metastasis, without impacting primary tumor formation and growth.

Blood was collected from vehicle- and SB203580-treated counterparts, during the 3-6 week post-implantation period, and quantified the number of circulating tumor cells (CTCs) isolated from blood and cultured as RFP-positive colonies. Strikingly, while CTCs were able to be recovered from vehicle-treated mice, even 3 weeks post-implantation, no CTCs were recovered from SB203580-treated mice until week 5 and, in week 6, very few CTCs were recovered from SB203580-treated mice (FIG. 15E).

Interestingly, both p38 and FOXC2 have been implicated in tumor angiogenesis (Yoshizuka et al., 2012; Kume et al., 2012). However, the analyses of microvessel density—quantified by CD31 immunohistochemistry and image analysis—did not reveal significant differences in the vasculature of primary tumors from SB203580-treated mice compared to vehicle-treated counterparts, which might have explained the lack of CTCs in SB203580-treated mice. These findings suggest that p38 inhibition compromises the intravasation of CTCs.

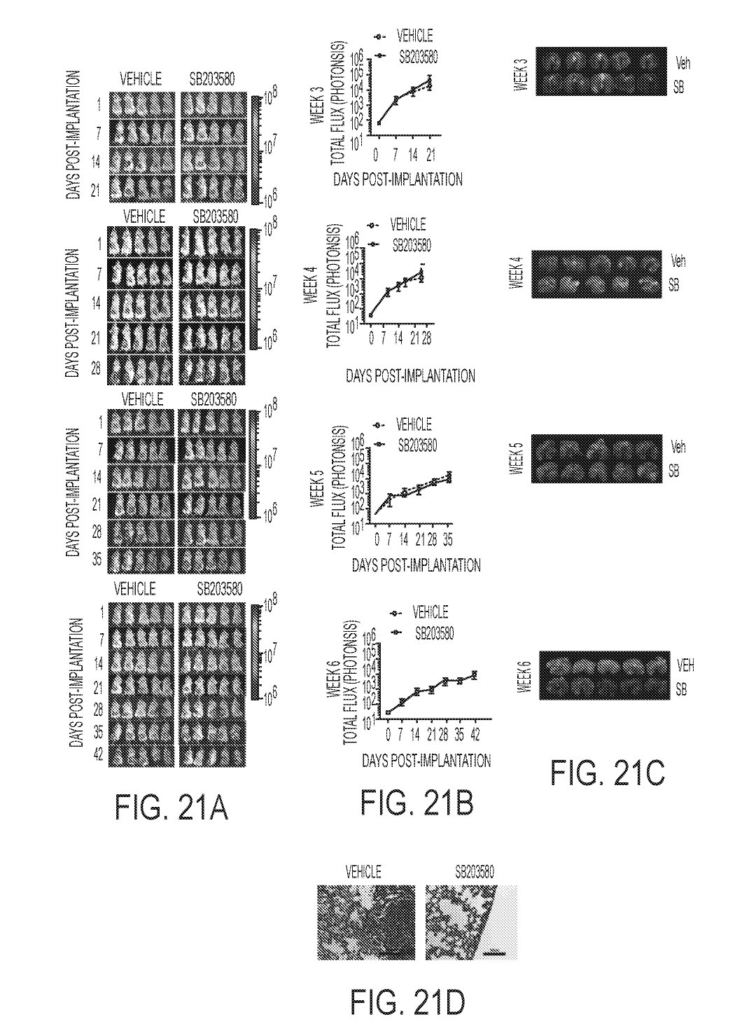

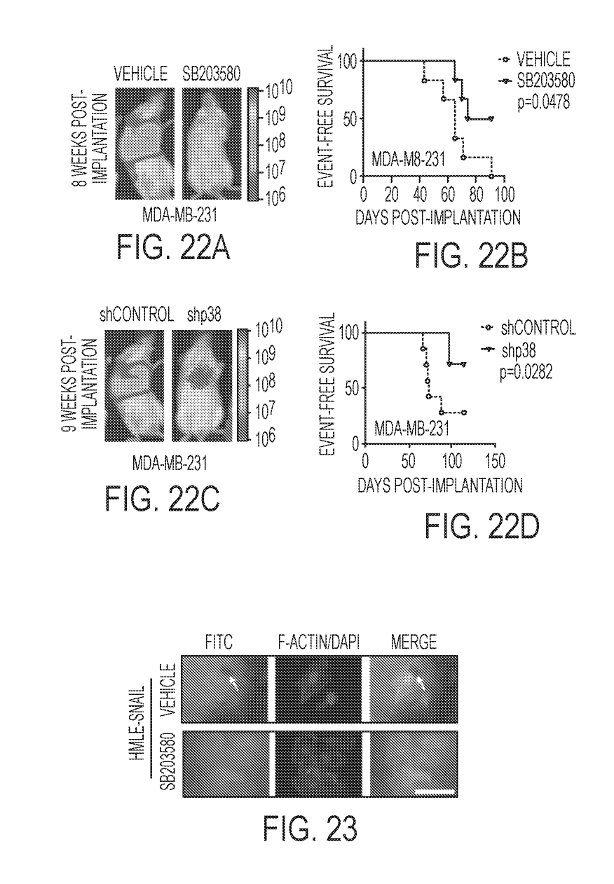

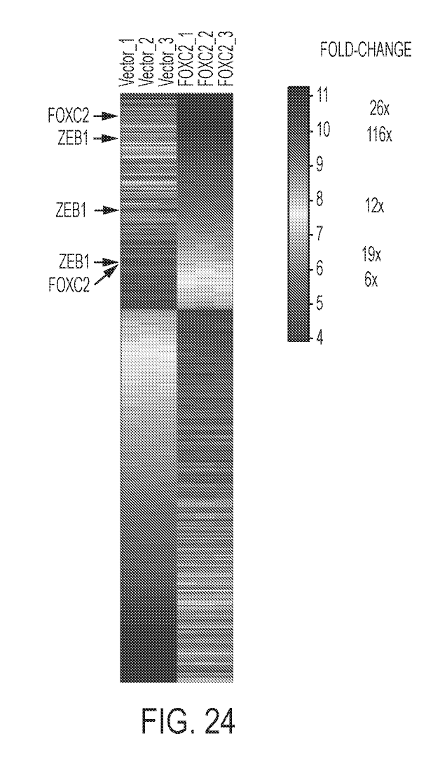

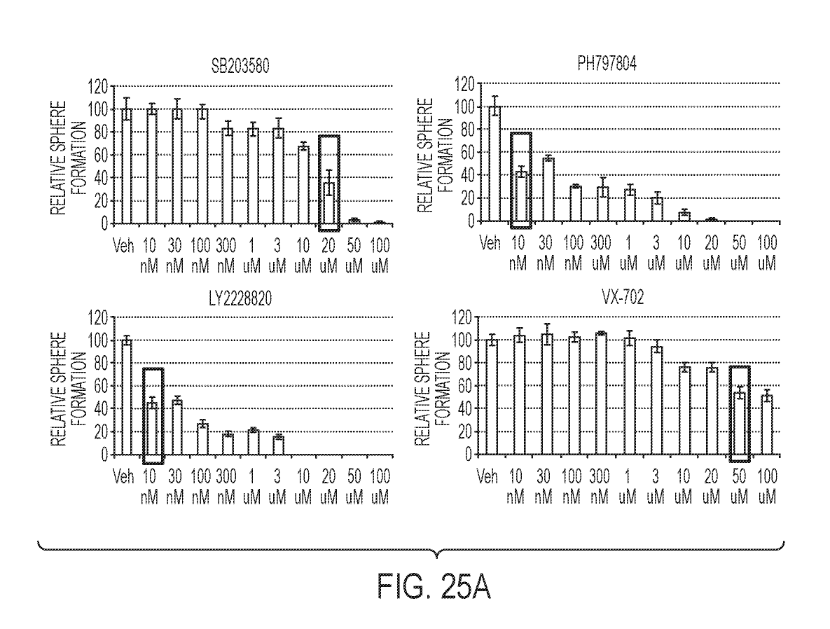

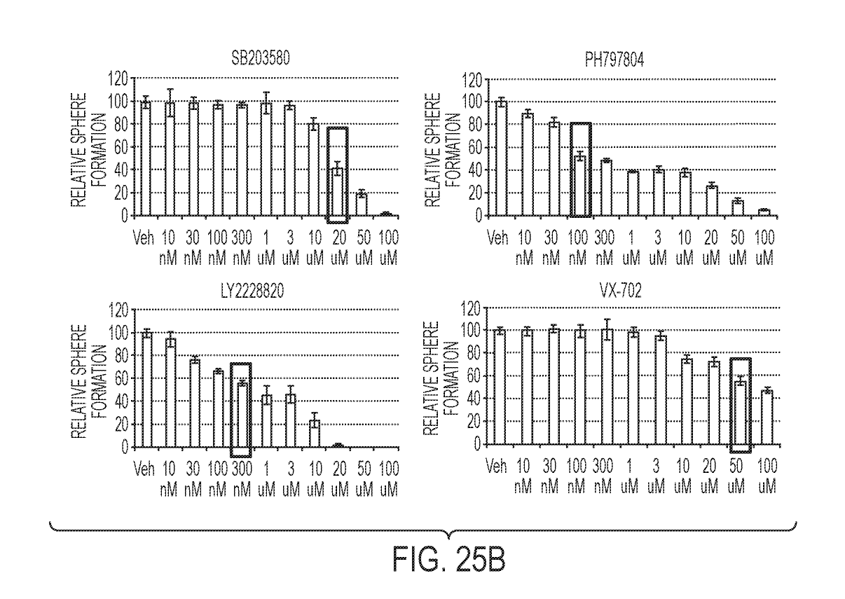

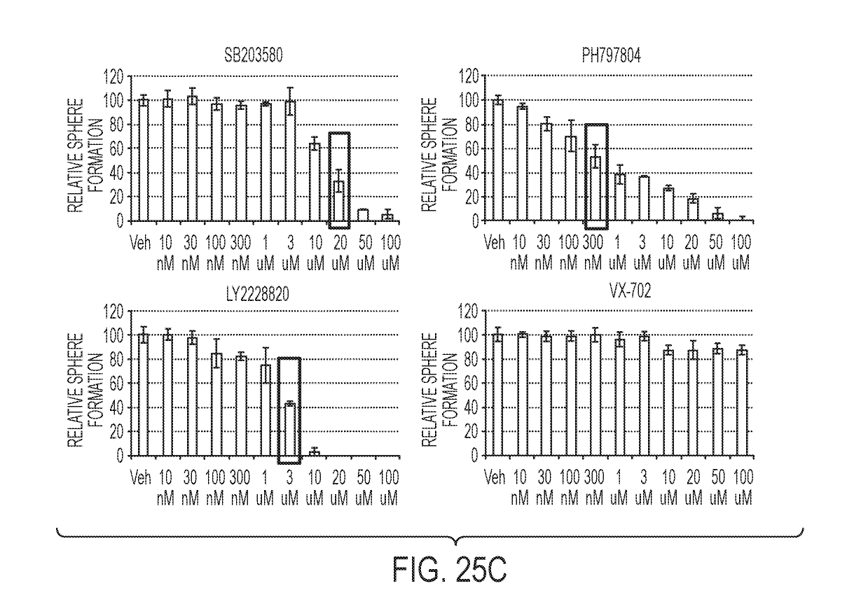

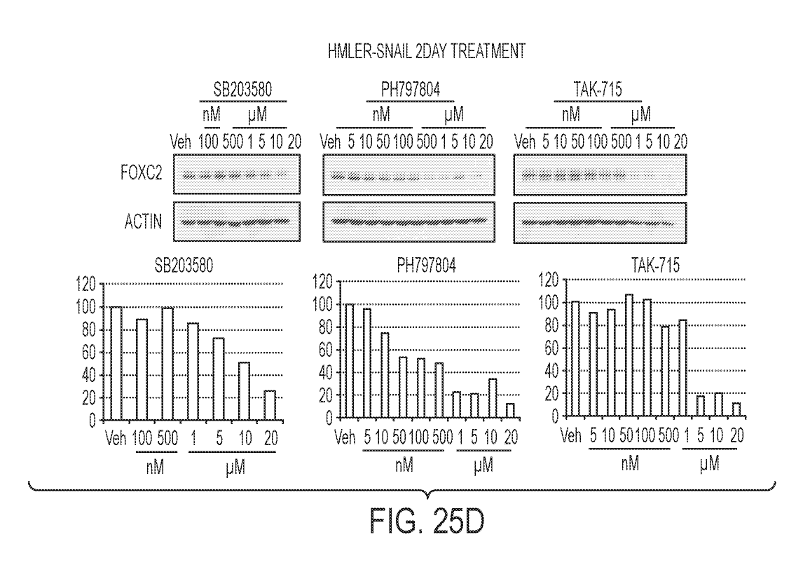

Since many breast cancer patients harbor occult micrometastases, at the time of diagnosis, and metastasis is often attributed to the systemic dissemination of tumor cells before or during surgical resection of the primary tumor (Pantel et al., 1999; Fehm et al., 2008), it was examined whether p38 inhibition could also prevent colonization and progression to macrometastasis. For this, an experimental metastasis model was employed, which circumvents the early steps of the metastatic cascade. Luciferase-labeled MDA-MB-231 cells were injected via the tail vein of NOD/SCID mice, and the emergence of lung metastases were monitored using bioluminescence. In concordance with the observations using the 4T1 model, injection of SB203580 daily, beginning 48 h post-implantation, significantly reduced the metastatic burden and extended event-free survival compared to vehicle-treated mice (FIGS. 21A, 21B). To eliminate the possibility of “off-target” effects, mice were intravenously injected with MDA-MB-231 cells expressing either control shRNA or p38 shRNA. These data suggest that p38 inhibition could also curtail colonization at the distant site. It was concluded that whereas p38 inhibition does not significantly affect primary tumor growth, it negatively impacts CTC numbers, lung colonization and, ultimately, metastasis.

p38 Inhibition Compromises EMT and Stem Cell Traits In Vitro.

The above findings suggested that p38 might regulate specific cellular attributes associated with the ability to navigate/complete the invasion-metastasis cascade (Valastyan et al., 2011). Since FOXC2 knockdown prevents EMT and the acquisition of stem cell properties, the impact of p38 inhibition on these intertwined processes was investigated.

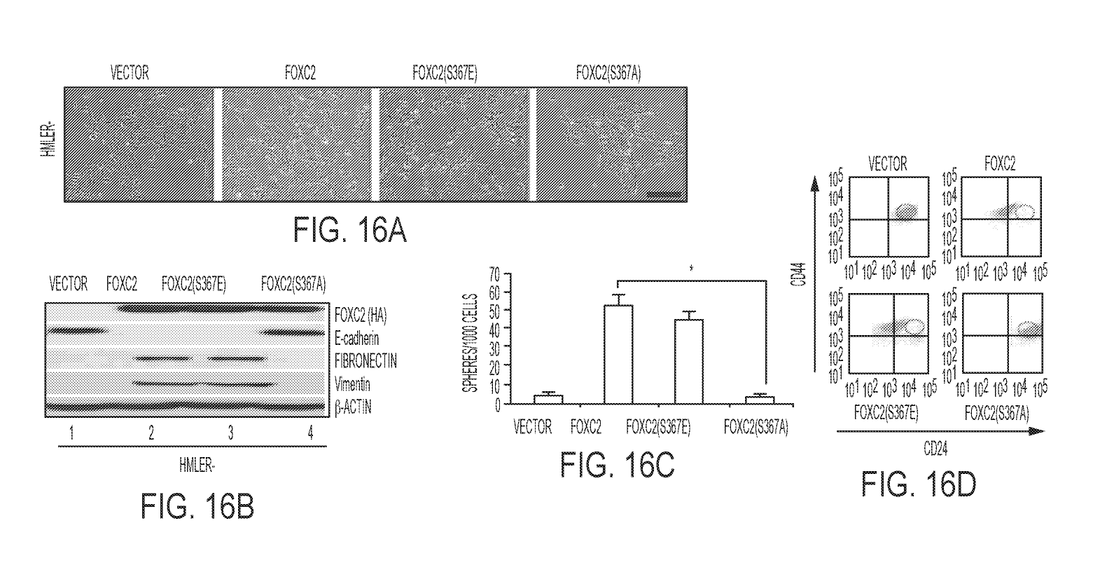

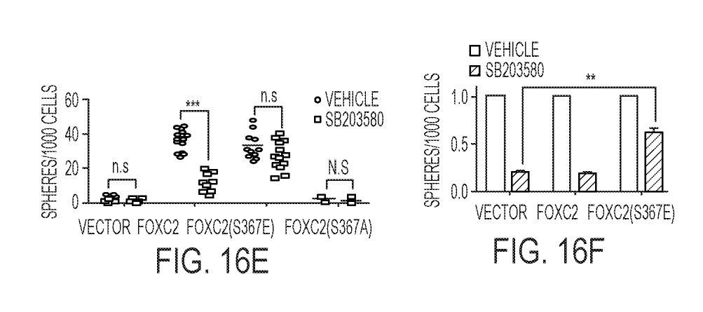

To ascertain whether inhibiting p38 impedes the initiation of EMT, two dynamic models of EMT induction were utilized. First, MCF10A immortalized human mammary epithelial cells were treated with TGFβ1 which elicits EMT. It was found that inhibition of p38, by concomitant SB203580 treatment, suppresses the upregulation of FOXC2 and mesenchymal markers (fibronectin, vimentin) and prevents downregulation of the epithelial marker E-cadherin in TGFβ1-treated cells (FIG. 16A). Second, an inducible EMT system was used wherein a fusion protein, comprising the EMT-inducing transcription factors Snail or Twist and the estrogen-binding domain of the estrogen receptor (ER), is stably expressed in epithelial HMLE cells (HMLE-Snail-ER, HMLE-Twist-ER). Addition of the ER-ligand tamoxifen (4-OHT) promotes nuclear translocation of the Snail-ER and Twist-ER proteins and elicits EMT. While 4-OHT treatment alone instigated EMT (FIG. 16B, lanes 4, 5), HMLE-Snail-ER cells, concurrently exposed to 4-OHT and SB203580, failed to undergo EMT or upregulate FOXC2 (FIG. 4B, lanes 9, 10). Additionally, 4-OHT-treated HMLE-Snail-ER and HMLE-Twist-ER cells failed to acquire sphere-forming potential (FIG. 16C) and the stem cell-associated CD44^{high}/CD24^{low} marker profile (FIG. 16D) following SB203580 exposure. Similarly, p38 shRNA abolished the capacity of HMLE-Twist-ER cells to undergo EMT (FIG. 4E, compare lanes 4 to 8) and to form spheres (FIG. 16F) in response to 4-OHT treatment. Collectively, these results suggested that p38 inhibition compromises the initiation of EMT and the acquisition of stem cell attributes elicited by well-known EMT-inducers.

It was next ascertained whether p38 inhibition compromises the maintenance of the established EMT phenotype. Indeed, it was found that exposure of mesenchymal HMLE-Snail, HMLE-Twist, MDA-MB-231 and SUM159 cells to SB203580 elicited various degrees of inhibition of mesenchymal and stem cell traits (FIGS. 16G-16L). Thus, SB203580 treatment altered the expression of EMT markers (FIG. 16G), and substantially reduced sphere-formation (FIG. 16H) and the percentage of cells displaying the CD44^{high}/CD24^{low} antigenic profile (FIG. 16I), relative to vehicle-treated cells.

Consistent with the fact that EMT confers increased migratory potential, SB203580 markedly reduced the migratory capacity of mesenchymal cells in a scratch/wound-healing assay (FIG. 16J). Moreover, HMLE cells expressing shRNAs targeting p38 or FOXC2, displayed markedly reduced migratory capacity compared to control shRNA counterparts (FIG. 16K). These results argue for an important role of p38-mediated phosphorylation of FOXC2 in eliciting wound closure, consistent with our earlier observations that FOXC2 nuclear staining is lacking at the leading edge of wounded SB203580-treated HMLE monolayers (FIG. 13F) that failed to repopulate the void created by the scratch. The effects of p38 inhibition on invasive potential, which strongly correlates with the formation of invadopodia, was investigated. These specialized actin-based cell membrane protrusions secrete matrix metalloproteinases, enabling degradation of the nearby extracellular matrix (Eckert et al., 2011). The formation of invadopodia was quantified by assessing the ability of cells to degrade FITC-conjugated gelatin. Using this assay, it was found that vehicle-treated HMLE-Snail and HMLE-Twist cells degraded the underlying extracellular matrix within 16 h, whereas SB203580-treated counterparts failed to do so to the same degree (FIG. 16L; 22). Collectively, these results suggest that p38 signaling not only plays a critical role in the initiation of the EMT program, instigated by various EMT-inducers, but also that it actively sustains the maintenance of EMT and the stem cell attributes it confers.

p38 Controls EMT and Stem Cell Traits Via FOXC2.

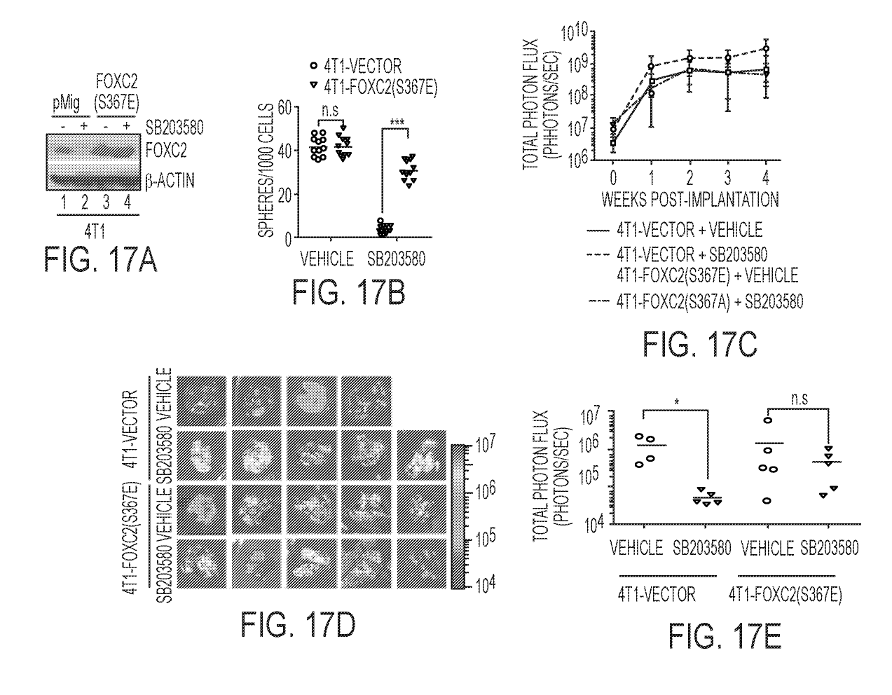

Having established that FOXC2 is a p38 substrate, it was next determined whether phosphorylation of FOXC2(S367) is critical for the acquisition of EMT and stem cell traits. For this, phosphomimetic FOXC2(S367E) and non-phosphorylatable FOXC2(S367A) mutants were generated and their ability to bestow mesenchymal and stem-cell traits relative to wild-type FOXC2 (referred to as FOXC2) was evaluated. Both FOXC2 and FOXC2(S367E) elicited EMT in Ras-transformed HMLE cells (HMLER), as evidenced by the acquisition of an elongated, spindle-shaped morphology (FIG. 17A), loss of E-cadherin and gain of mesenchymal markers (FIG. 17B, lanes 2 and 3). Conversely, the FOXC2(S367A) mutant-even though it was surprisingly expressed at comparable levels-failed to induce EMT (FIGS. 17A and B, lane 4). Furthermore, ectopic expression of either FOXC2 or the FOXC2(S367E) mutant enhanced the sphere-forming potential of HMLER cells (FIG. 17C) and promoted a shift towards the CD44^{high}/CD24^{low} antigenic profile²¹ (FIG. 17D). Moreover, whereas SB203580 reduced—as anticipated—the sphere-forming capacity of HMLER-FOXC2 cells, HMLER-FOXC2(S367E) cells retained the ability to form spheres even in the presence of SB203580 (FIG. 17E). In the aforementioned assays, the non-phosphorylatable FOXC2(S367A) mutant did not promote sphere formation (FIGS. 17C and E) and was associated with the CD44^{low}/CD24^{high} epithelial cell-surface marker profile (FIG. 17D). Finally, whereas SB203580 inhibited the migration of HMLER-vector (>70%) and HMLER-FOXC2 cells (>70%), HMLER-FOXC2(S367E) cells exhibited only a modest decrease (<30%) in wound-closure (FIG. 17F). Collectively, these findings demonstrated that p38-mediated phosphorylation of the S367 residue of FOXC2 empowers it to confer combined EMT and stem cell attributes in vitro.

p38-Mediated Phosphorylation of FOXC2 Promotes Metastasis.

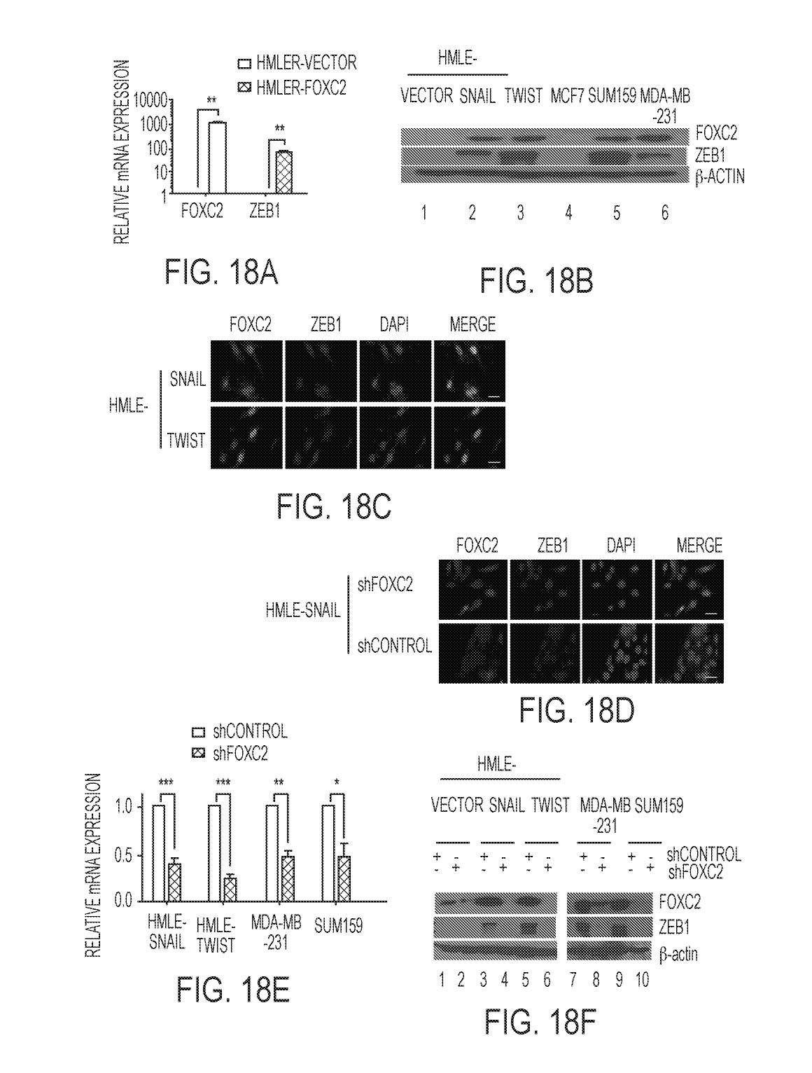

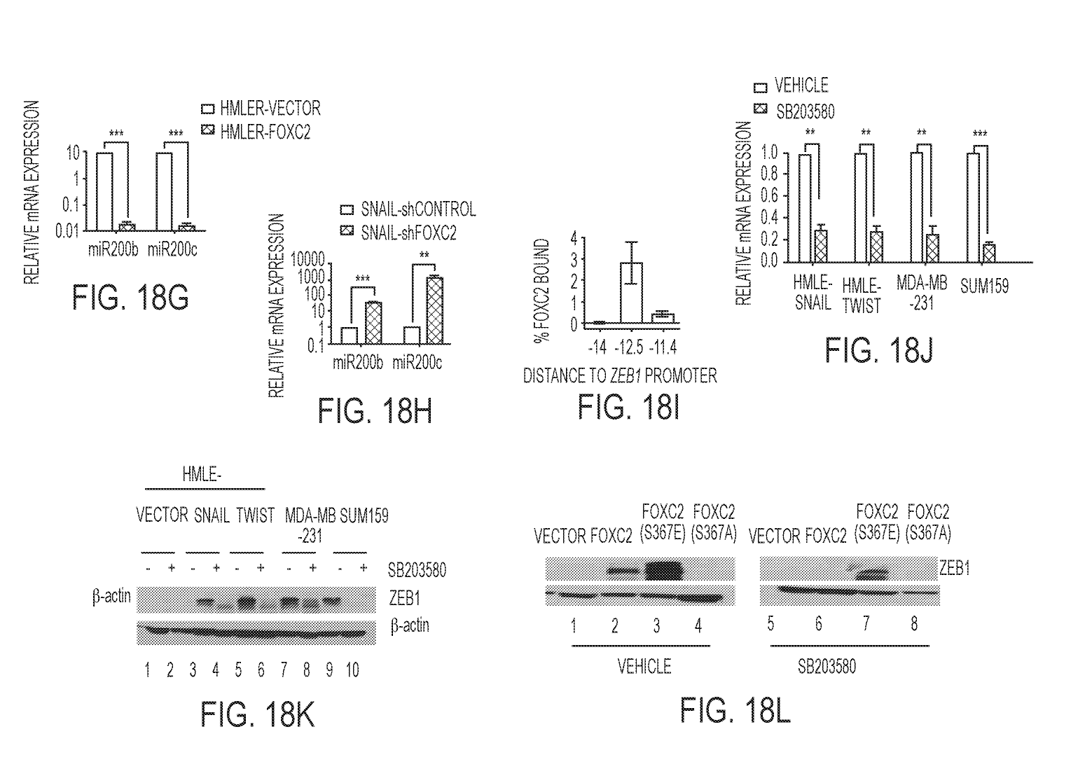

It was next tested whether p38-mediated phosphorylation of FOXC2 enhances its ability to confer metastatic competence. First, it was demonstrated that whereas SB203580 treatment of vector-transduced 4T1 cells abolished endogenous Foxc2 protein levels, the phosphomimetic FOXC2(S367E) levels remained unaltered (FIG. 18A). Accordingly, whereas SB203580 compromised the sphere-forming efficiency of vector-transduced 4T1 cells, 4T1-FOXC2(S367E) cells were refractory to p38 inhibition (FIG. 18B).

Next, luciferase-labeled 4T1 cells, expressing empty vector or FOXC2(S367E), were orthotopically implanted into the mammary fat-pad of BALB/c mice, and these mice were subsequently treated with SB203580. Similar to the earlier findings, there were no significant differences in primary tumor growth following SB203580 treatment in mice harboring 4T1-vector or 4T1-FOXC2(S367E) cells (FIG. 18C). Moreover, the incidence of lung metastases in SB203580-treated mice, harboring 4T1-vector cells, was reduced by >20-fold compared to vehicle-treated counterparts (FIGS. 18D and 18E). In sharp contrast, numerous lung nodules could be detected in mice harboring 4T1-FOXC2(S367E) cells, and most importantly, SB203580 failed to significantly reduce the metastatic burden (FIGS. 18D and 18E), compared to the vehicle-treated 4T1-vector and 4T1-FOXC2(S367E) counterparts.

p38-Mediated Phosphorylation of FOXC2 Regulates ZEB1 Expression.

To identify potential downstream mediators of p-p38 and FOXC2, the previous microarray data (GEO accession: GSE44335) from HMLER-FOXC2 cells relative to HMLER-vector counterparts was analyzed, and the transcription factor ZEB1 was identified as one of the highly upregulated genes (116-fold) in HMLER-FOXC2 cells (FIG. 23). First, the elevated levels of FOXC2 and ZEB1 transcripts were confirmed in HMLER-FOXC2 cells versus HMLER-vector cells by qRT-PCR (FIG. 19A). Furthermore, immunoblotting revealed a positive correlation between elevated FOXC2 and ZEB1 protein levels in cells induced to undergo EMT and CSC-enriched cell lines (FIG. 19B). At the cellular level, immunofluorescence demonstrated that FOXC2 and ZEB1 co-localize in the nuclei of HMLE-Snail and HMLE-Twist cells (FIG. 19C), and that FOXC2 knockdown elicits a marked decrease in ZEB1 staining intensity (FIG. 19D). Accordingly, shRNA-mediated suppression of FOXC2 in a panel of cell lines yielded a >50-fold decrease in ZEB1 mRNA (FIG. 19E) and a >80% reduction in ZEB1 protein levels (FIG. 19F). Furthermore, consistent with the reciprocal regulation of ZEB1 and miR-200 family members (Burk et al., 2008), decreased levels of miR-200b and miR-200c were observed in HMLER-FOXC2 cells, compared to vector-transduced counterparts (FIG. 19G). Conversely, a >50-fold increase as found in miR-200b, and a >1000-fold increase in miR-200c, following FOXC2 knockdown compared to control shRNA-transduced counterparts (FIG. 19H). To ascertain whether FOXC2 directly regulates miR-200 or ZEB1 expression, the promoter regions of both miR-200 clusters on chromosomes 1 and 12 as well as the ZEB1 promoter were analyzed, and a conserved FOXC2-binding element within the ZEB1 promoter was identified. Indeed, using chromatin immunoprecipitation, it was found that FOXC2 preferentially binds to a region around 12.5 kb (−12.5 kb) upstream of the ZEB1 transcription start site (FIG. 19I), thus confirming that FOXC2 is a direct transcriptional regulator of ZEB1. Consistent with the findings that FOXC2 transcriptionally regulates ZEB1, and that p38 inhibition diminishes FOXC2 expression, ZEB1 mRNA and protein levels were reduced by >60-fold following SB203580 treatment compared to vehicle-treated cells (FIGS. 19J and 19K). Furthermore, in support of the notion that p38-mediated phosphorylation of FOXC2 regulates ZEB1 expression, elevated ZEB1 protein levels were only detected in HMLER-FOXC2 and HMLER-FOXC2(S367E) cells (FIG. 19L, lanes 2 and 3), but not in HMLER-FOXC2(S367A) cells (FIG. 19L, lane 4). Moreover, when HMLER cells expressing the FOXC2 variants were treated with SB203580, ZEB1 protein levels were decreased in HMLER-FOXC2 cells (FIG. 19L, lane 6) but remained relatively high in HMLER-FOXC2(S367E) cells (FIG. 19K, lane 7). Collectively, these findings demonstrated that p38-mediated phosphorylation of FOXC2 directly regulates the expression of ZEB1.

It was previously reported that FOXC2 is a critical regulator of EMT, stem cell properties and metastatic competence. However, the fact that FOXC2 is a transcription factor renders it inherently difficult to inhibit pharmacologically (Darnell et al., 2002). Herein, the serine/threonine-specific kinase p38 was identified as a druggable upstream regulator of FOXC2 function. Phosphorylation of FOXC2 by p38 at S367 regulates FOXC2 protein stability, promotes expression of its downstream target ZEB1, and modulates its ability to confer EMT properties and stem cell attributes in vitro and metastatic competence in vivo.

The findings support the existence of two CSC-subtypes: CSCs with tumor-initiating capabilities that underpin primary tumor growth, and CSCs endowed with dual tumor-initiating and metastatic competencies that fuel metastatic outgrowths. The finding that inhibition of p38-FOXC2 signaling impedes only metastasis and not the primary tumor, suggests that the p38-FOXC2 pathway could serve to distinguish tumor-initiating CSCs from metastasis-competent CSCs.

In conclusion, this study links p38-mediated phosphorylation of FOXC2 to the regulation of its downstream target ZEB1, EMT, stem cell traits and metastatic competence, and attests to the potential utility of p38-inhibitors to attenuate FOXC2-dependent metastasis.

### Example 4—Materials and Methods

Cell Culture:

Immortalized human mammary epithelial (HMLE) cells expressing empty vector (pWZL), Snail, Twist, Goosecoid (GSC), or an activated form of TGFβ1, V12H-Ras-transformed HMLE (HMLER) and HMLER-FOXC2 cells were maintained as previously described (Elenbaas et al., 2001) For 4-hydroxy-tamoxifen (4-OHT) treatment, HMLE-Snail-ER or HMLE-Twist-ER cells were exposed to 20 nM 4-OHT for the indicated number of days. MCF7, MDA-MB-231 and SUM159 human breast cancer cells and the 4T1 mouse mammary carcinoma cells were cultured as described. Human non-tumorigenic MCF10A cells were cultured as described (Hollier et al., 2013), and treated with 2.5 ng/ml TGFβ1 for 3 days to elicit EMT. Cells were cultured for 24 h before addition of 20 μM SB203580 (Calbiochem, San Diego, Calif., USA).

Plasmids, shRNA and Transduction:

The expression vectors encoding HA-tagged p38 (HA-p38) and kinase-dead p38 (HA-p38-DN) have been described (Kawano et al., 2003). FOXC2 was PCR-amplified from pBabePuro-FOXC2 and subcloned into pcDNA3.1/myc-His vector. FOXC2-mutant constructs were generated by site-directed mutagenesis and subcloned into the retroviral vector MSCV-IRES-GFP. The primers used were: FOXC2(S367E) forward, 5′-cgagcggccccacggagcccctgagcgctctcaacc-3′; reverse, 5′-ggttgagagcgctcaggggctccgtggggccgctcg-3′ and FOXC2(S367A) forward, 5′-cgagcggccccacggcacccctgagcgctctcaacc-3′; reverse, 5′-ggttgagagcgctcaggggtgccgtggggccgctcg-3′.

To suppress p38 and FOXC2 expression, the shRNA-expressing lentivirus system was used (Open Biosystems, Huntsville, Ala., USA). The shRNA sequences targeting p38 and FOXC2 were TTCACAGCTAGATTACTAG and CCTGAGCGAGCAGAATTACTA respectively. shRNA targeting firefly luciferase (shControl) was used as a control. Lentiviral or retroviral transduction of target cells was performed as described previously (Stewart et al., 2003). Stable transductants were selected in 2 μg/ml puromycin.

Immunoblotting, Immunofluorescence and Antibodies:

Immunoblotting and immunofluorescence (Mani et al., 2008) were performed as previously described. Primary antibodies were as follows: β-actin (Sigma, St Louis, Mo., USA; A3853), mouse anti-human FOXC2 (Dr. Naoyuki Miura, Hamamatsu University School of Medicine, Japan), goat polyclonal anti-FOXC2 (Santa Cruz Biotechnology, Dallas, Tex., USA; sc-21397), p-p38 (Cell Signaling, Danvers, Mass., USA; 4511), p38 (Cell Signaling; 9211), E-cadherin (BD Biosciences, San Jose, Calif., USA; 61081), fibronectin (BD Biosciences; 610077), vimentin (Novus Biologicals, Littleton, Colo., USA; NB200-623), ZEB1 (Novus Biologicals; NBP1-05987), and HA (Covance, Princeton, N.J., USA; MMS-101P).

Co-Immunoprecipitation:

Cell lysates were incubated with antibodies overnight at 4° C. Protein A/G-agarose beads (50 μl; Pierce, Waltham, Mass., USA) were added for 12 h at 4° C. The beads were washed with ice-cold radioimmunoprecipitation buffer (Sigma) containing protease and phosphatase inhibitors (Roche, Nutley, N.J., USA). Bound proteins were eluted by boiling in sample buffer, resolved by SDS-PAGE and analyzed by immunoblotting.

In vitro kinase assays: Glutathione-S-transferase-(GST)-tagged FOXC2 truncation mutants were subcloned into pGEX-6P-1 and expressed in E. coli. Cell lysates were cleared by centrifugation and the GST-FOXC2 fusion proteins absorbed on glutathione-sepharose-4B beads (Sigma) for 2 h at 4° C. The beads were washed with lysis buffer and the GST-FOXC2 fusion proteins eluted with reduced glutathione. The eluates (200 ng) were incubated with 100 ng of recombinant active p38a (Invitrogen, Grand Island, N.Y., USA; PV3304) in the presence of 60 mM MgCl₂, 60 μM ATP, 50 mM Tris-HCl (pH 7.5), 12 mM DTT, protease and phosphatase inhibitors (Roche), and 0.7 μCi of [γ-³²P]ATP, at room temperature for 30 min.

qRT-PCR:

qRT-PCR was performed using SYBR Green (Applied Biosystems, Waltham, Mass., USA) for mRNAs and Taqman (Applied Biosystems) for microRNAs as described previously (Hollier et al., 2013).

Chromatin Immunoprecipitation:

Chromatin immunoprecipitation was performed as described previously (Hollier et al., 2013).

Assays for EMT and Stem Cell Properties:

Quantification of invadopodia (Eckert et al., 2011), scratch/wound-healing assays (Sarkar et al., 2015) fluorescence-activated cell sorting (FACS), and sphere-formation assays (Mani et al., 2008) were conducted as described previously.

Animal Studies:

NOD/SCID and BALB/c mice were purchased from the Jackson Laboratory (Bar Harbor, Me., USA). To examine primary tumor formation and spontaneous metastasis, luciferase-labeled 4T1 cells (1×10⁴ or 5×10⁴) were injected into the inguinal mammary fat-pad of BALB/c mice. For experimental metastasis studies, 0.5×10⁶ luciferase-labeled MDA-MB-231 cells were injected into NOD/SCID mice via the tail vein. Thereafter, vehicle or SB203580 (0.2 μmols in 100 μl per ˜20 g mouse) was administered once daily subcutaneously. Mice were assessed weekly for tumor growth and metastasis via subcutaneous injection of D-Luciferin (150 mg/kg; Caliper LifeSciences, Hopkinton, Mass., USA) and bioluminescent imaging (IVIS imaging system 200 series; Xenogen Corporation, PerkinElmer, Waltham, Mass., USA). Primary tumor size was measured with a caliper as the product of two perpendicular diameters (mm²). At the indicated timepoints, primary tumors and lungs were surgically excised, imaged and processed for histology.

Enumeration of CTCs:

Mice were orthotopically injected with red fluorescent protein (RFP)/luciferase-labeled 4T1 cells. Starting week 3 post-injection, blood was collected, via cardiac puncture, in EDTA-coated tubes and treated with Ammonium-Chloride-Potassium lysing buffer (Invitrogen). Cells were cultured in RPMI-1640 medium containing 10% fetal bovine serum and penicillin/streptomycin. RFP-positive colonies were counted after 3 days.

### Example 5—p38 MAPK Inhibitors Prevent Mesenchymal Stem Cell-Mediated Tyrosine Kinase Inhibitor Resistance in BCR-ABL+ ALL

Clinical Compound Screening Identified Agents that Diminish Imatinib-Induced MSC-Mediated Leukemic Cell Protection:

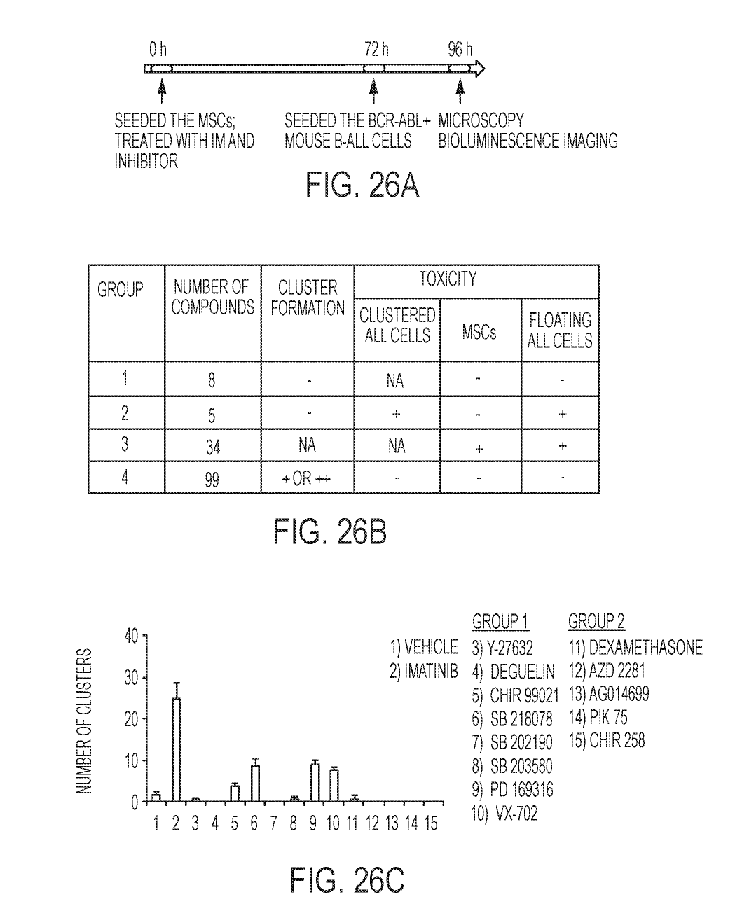

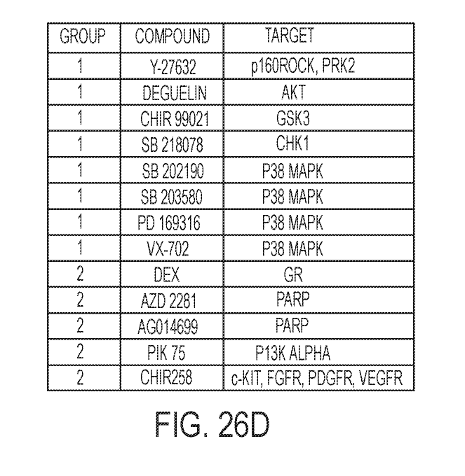

Previously, it was demonstrated that, imatinib treatment of MSC and leukemic cell co-cultures, induced morphological, molecular, and functional alterations in MSCs and that ALL cell clusters formed underneath the MSCs. It was also shown that, although BCR-ABL signaling was inhibited by imatinib, leukemic cells relied on MSC-mediated signaling for survival, which led to leukemic cell resistance to imatinib (Mallampati et al., 2015). In the current study, a library of 146 clinical compounds was screened to identify agents that could inhibit imatinib-induced MSC-mediated signaling in leukemic cells and disrupt leukemic cell clusters (FIG. 26A). The effectiveness of the compounds was determined in preventing cluster formation by microscopy and the toxicity of the compounds by bioluminescence imaging of co-cultured MSCs and leukemic cells.

On the basis of the screening results, these compounds were categorized into four groups (FIG. 26B and FIGS. 26A-D). Group 1 compounds (n=8) were not overtly toxic to either leukemic cells or MSCs but efficiently impeded formation of leukemic cell clusters with imatinib-pretreated MSCs. Group 2 compounds (n=5) in the presence of imatinib exhibited toxicity toward leukemic cells, including clustered leukemic cells, but showed no toxicity to MSCs. Group 3 compounds (n=34) were toxic to both MSCs and leukemic cells. Group 4 compounds (n=99) showed no toxicity to leukemic cells or MSCs and failed to disrupt imatinib-induced leukemic cell cluster formation.

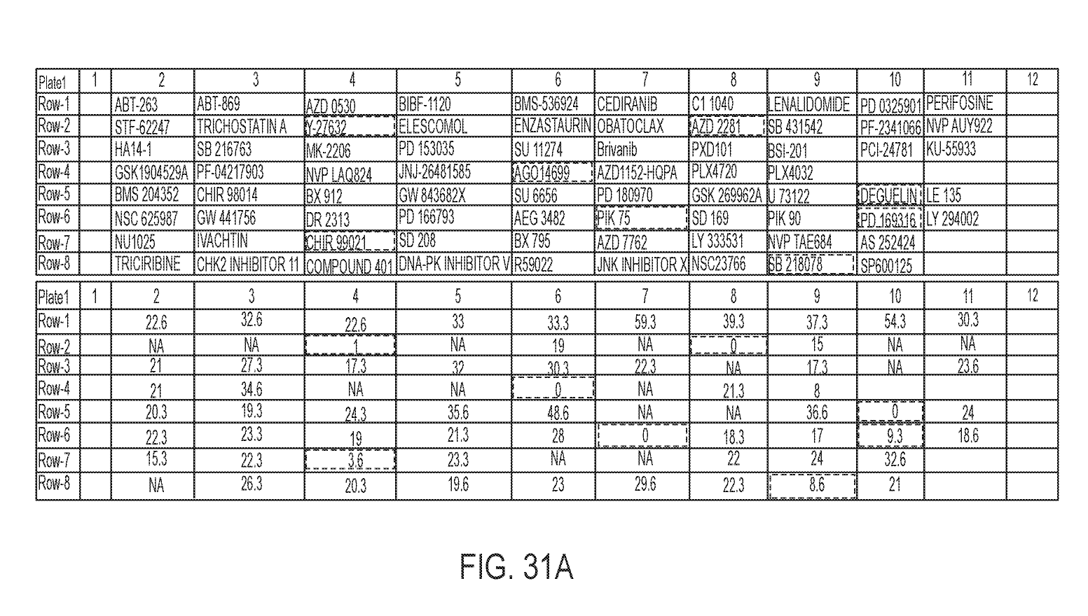

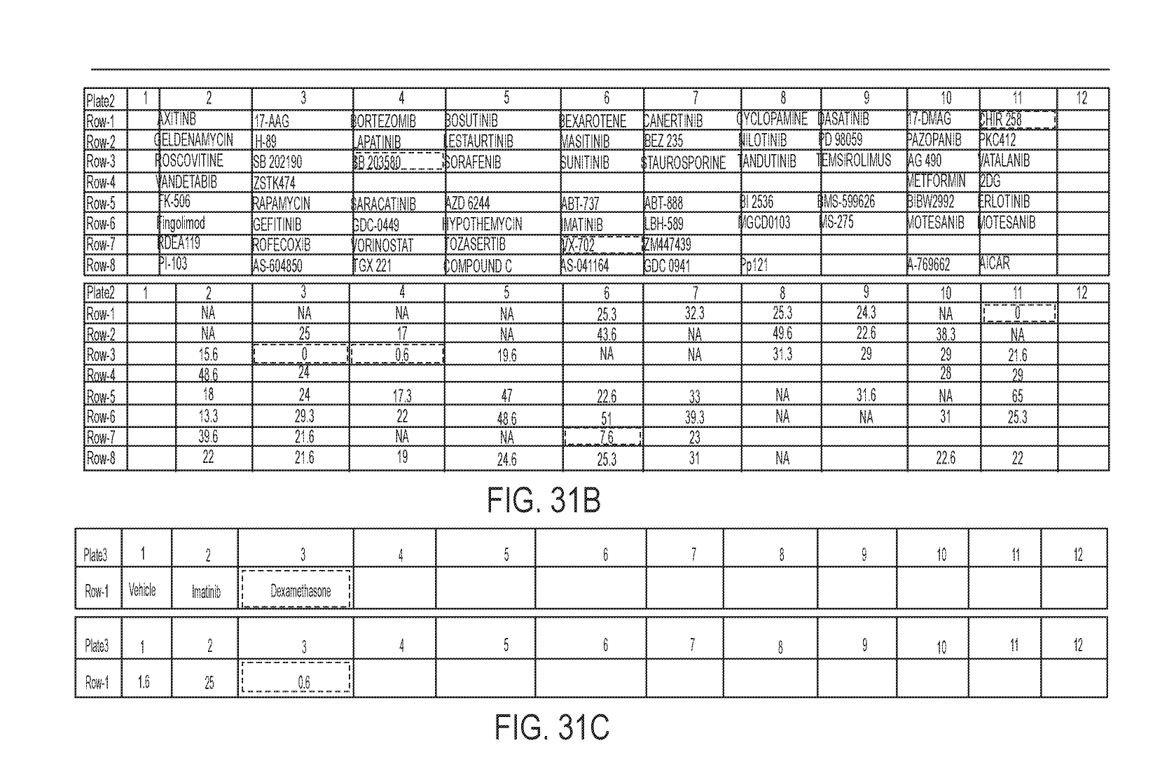

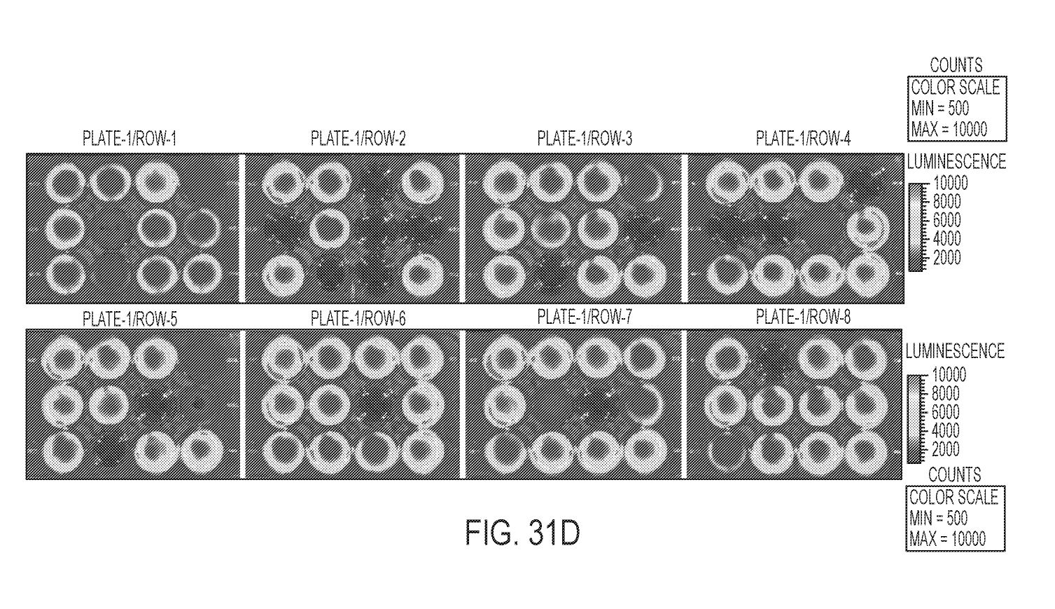

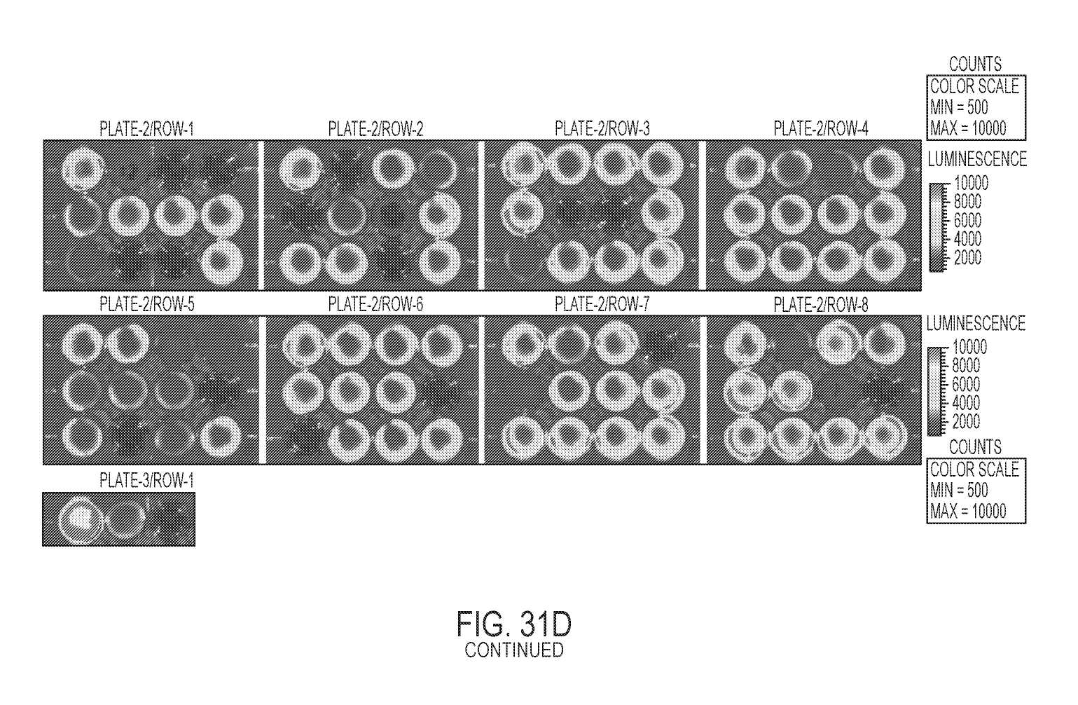

Given these results, the compounds from groups 1 and 2 were investigated. Group 1 compounds directly prevented imatinib-induced leukemic cell cluster formation by disrupting interactions between MSCs and leukemic cells, whereas group 2 compounds eliminated the leukemic cell clusters by eliciting toxicity toward clustered leukemic cells when imatinib was present (FIG. 26C and FIGS. 31A-D). Examination of signaling pathway status revealed that group 1 compounds targeted p160ROCK and PRK2 (compound Y-27632), AKT (compound DEGUELIN), GSK3 (compound CHIR99021), CHK1 (compound SB218078), and p38 MAPK (compounds SB202190, SB203580, PD169316, and VX-702). Group 2 compounds targeted glucocorticoid receptor (compound dexamethasone); PARP (compounds AZD2281 and AG014699); PI3K alpha (compound PIK75); and c-Kit, FGFR, PDGFR, and VEGFR (compound CHIR258; FIG. 26D). These findings suggested that targeted inhibition of any of several signaling pathways either in MSCs or leukemic cells could offset imatinib-induced off-target survival support.

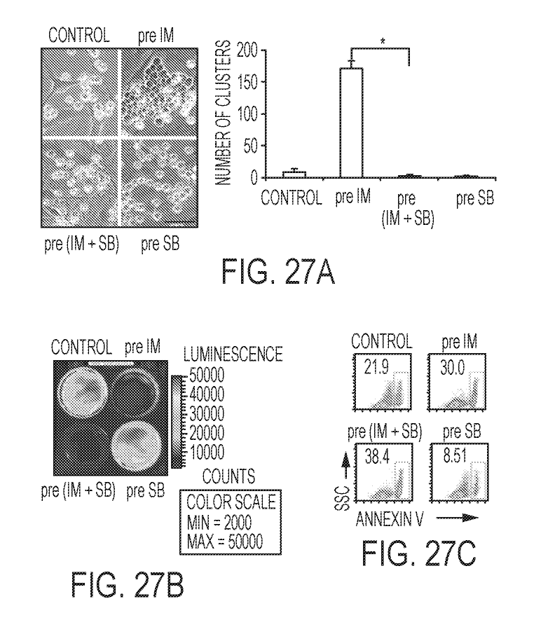

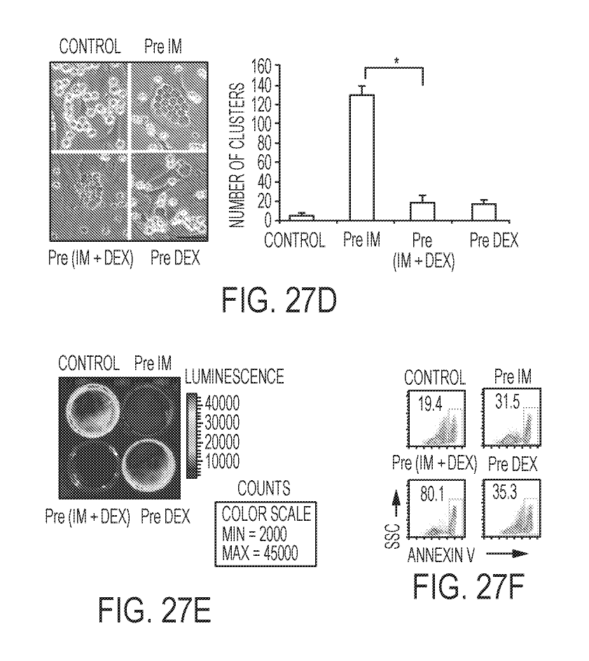

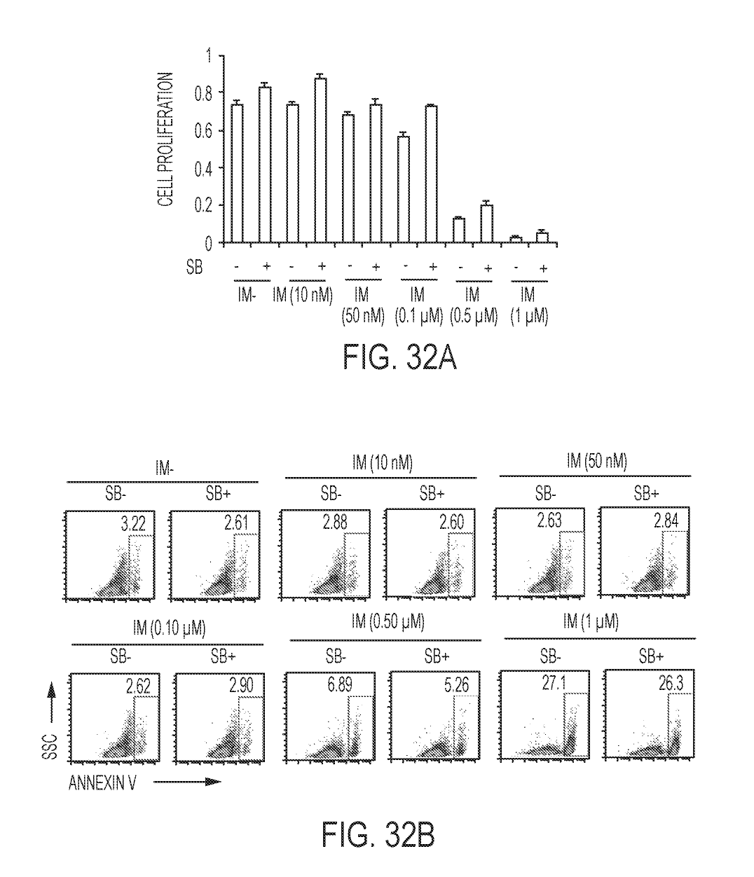

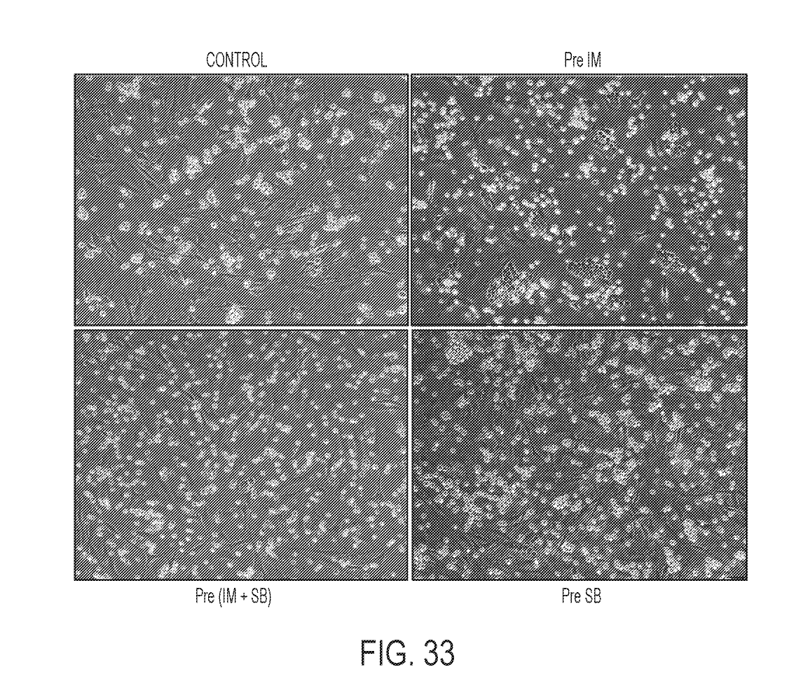

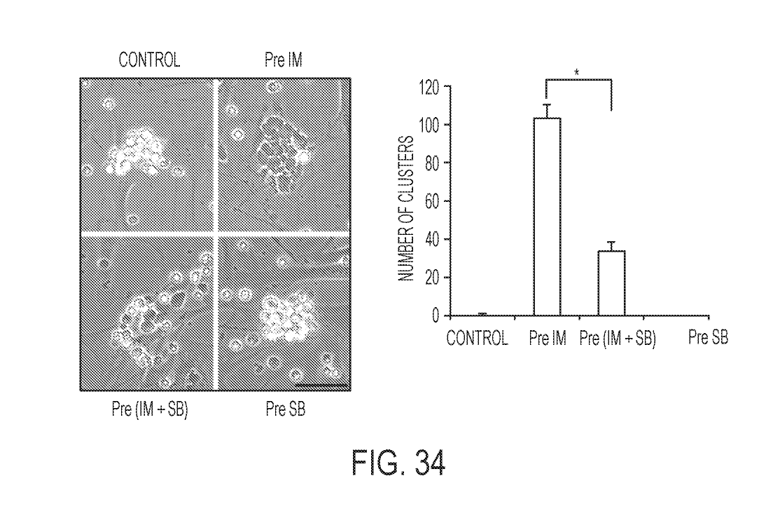

p38 MAPK inhibitor SB203580 and dexamethasone prevents imatinib-induced MSC-mediated support to leukemic cells: Four of the eight compounds from group 1 were p38 MAPK inhibitors, and the p38 MAPK inhibitor SB203580 potently inhibited the formation of imatinib-induced leukemic cell clusters (FIG. 26C). SB203580 alone did not affect proliferation or apoptosis of leukemic cells (FIG. 27B-C). Treating leukemic cells (cultured without MSCs) with both SB203580 and imatinib did not alter the imatinib effects on leukemic cells (FIG. 32). However, when MSCs were pretreated with imatinib prior to seeding leukemic cells, adding SB203580 completely prevented leukemic cell cluster formation (FIG. 27A and FIG. 33). Moreover, under these conditions, SB203580 and imatinib together diminished proliferation (FIG. 27B) and increased apoptosis of leukemic cells (FIG. 2C7). Similarly, it was found that SB203580 and imatinib in combination also prevented the formation of imatinib-induced clusters in BCR-ABL+ ALL cells from patients (FIG. 34). These findings suggest that combination treatment with SB203580 and imatinib could prevent imatinib-induced interactions between MSCs and leukemic cells, eliminate MSC support to leukemic cells, and sensitize leukemic cells to imatinib.

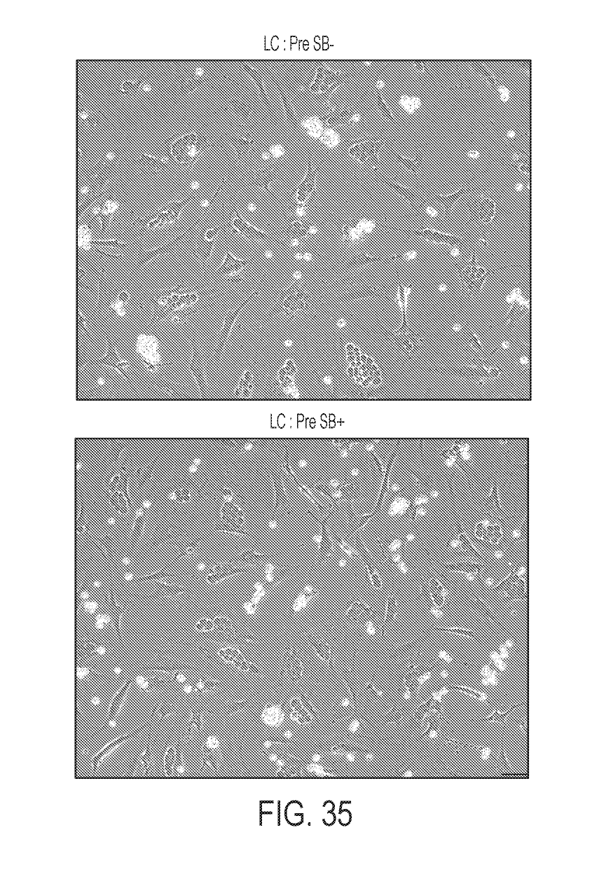

To determine whether SB203580's effect on leukemic cells had a role in preventing imatinib-induced leukemic cell clusters, leukemic cells were pre-treated with SB203580 and then seeded them onto Imatinib-pretreated MSCs. The efficiency in leukemic cell cluster formation did not differ significantly between SB203580-pretreated leukemic cells and the control, indicating that SB203580 prevents leukemic cell cluster formation through its effects on MSCs rather than affecting leukemic cells directly (FIG. 35). These findings were in agreement with our observation that imatinib-induced morphological alterations in MSCs from small and slender cells to large, polygonal cells were reversed when MSCs were treated with SB203580 (FIG. 33).

Dexamethasone is an integral component of CVAD or hyper-CVAD regimen used in the induction therapy for BCR-ABL+ ALL and is often combined with imatinib (Daver et al., 2015). In the initial screening of the 146 clinical compounds, dexamethasone eliminated leukemic cell clusters even at nanomolar concentrations, whereas other compounds were effective at micromolar concentrations. When combined with imatinib, dexamethasone efficiently targeted leukemic cell clusters (FIG. 27D). However, unlike SB203580, dexamethasone could not prevent the initial formation of imatinib-induced leukemic cell clusters. Dexamethasone plus imatinib significantly reduced leukemic cell proliferation and induced apoptosis, compared with either imatinib or dexamethasone alone (FIGS. 27E-F). These findings demonstrate that dexamethasone and imatinib together effectively target imatinib-induced leukemic cell clusters and eliminate imatinib-induced MSC-mediated support to leukemic cells.

SB203580 Co-Treatment Reverses Imatinib-Induced Molecular Alterations in MSCs:

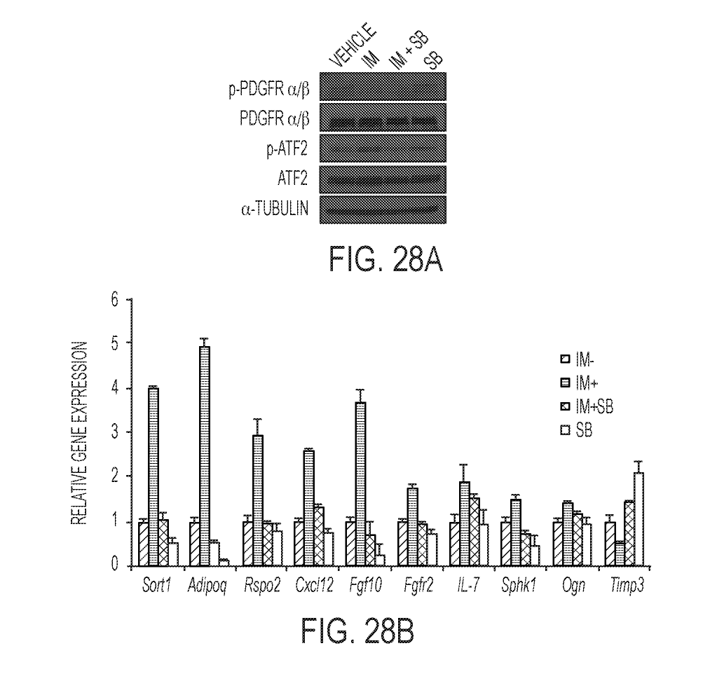

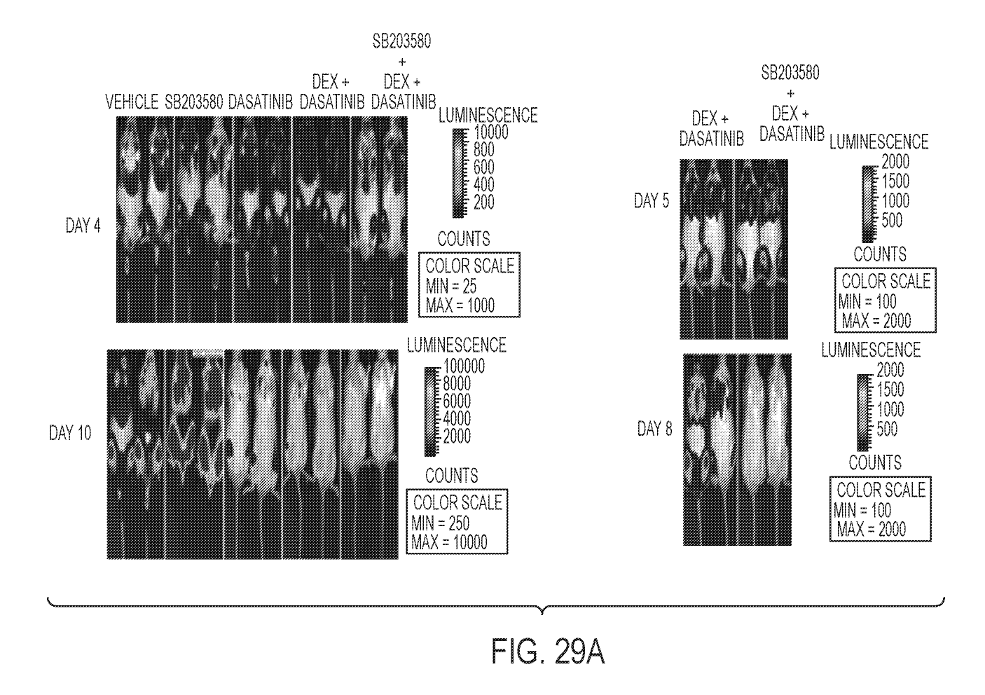

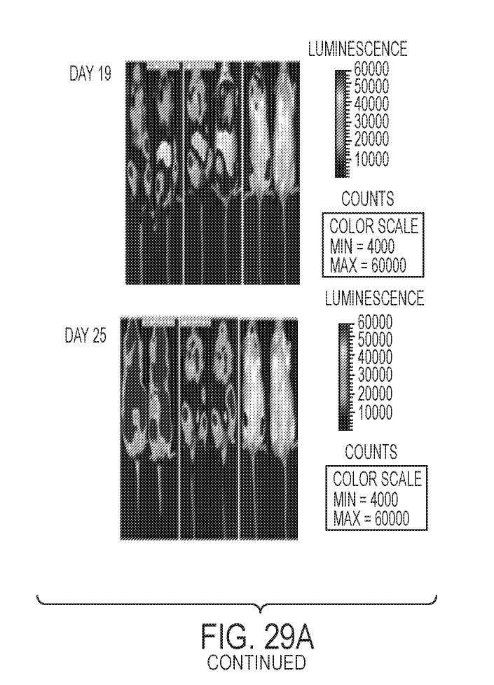

To determine whether imatinib treatment activated p38 MAPK in MSCs and whether the combination of SB203580 and imatinib could prevent activation of p38 MAPK, the phosphorylation of PDGFR-α/β, a known target of imatinib, and of ATF2, a downstream effector of p38 MAPK, was measured (Humphreys et al., 2013). Imatinib-treated MSCs had reduced phosphorylation of PDGFR-α/β, suggesting that imatinib treatment is effective in MSCs (FIG. 28A). MSCs treated with imatinib alone had higher phosphorylation of ATF2 than control, but this was not evident in MSCs treated with both imatinib and SB203580.

Previously, it was found that imatinib treatment altered expression of a network of genes associated with chemoattraction, adhesion, and cell survival (Mallampati et al., 2015). SB203580 and imatinib co-treatment reversed this imatinib-induced alteration of gene expression in MSCs (FIG. 28B). These gene expression results support our finding that p38 MAPK inhibitors eliminate off-target effects of imatinib.

Dasatinib+Dexamethasone+SB203580 Prevents TKI Resistance in Mouse Models of BCR-ABL+ ALL:

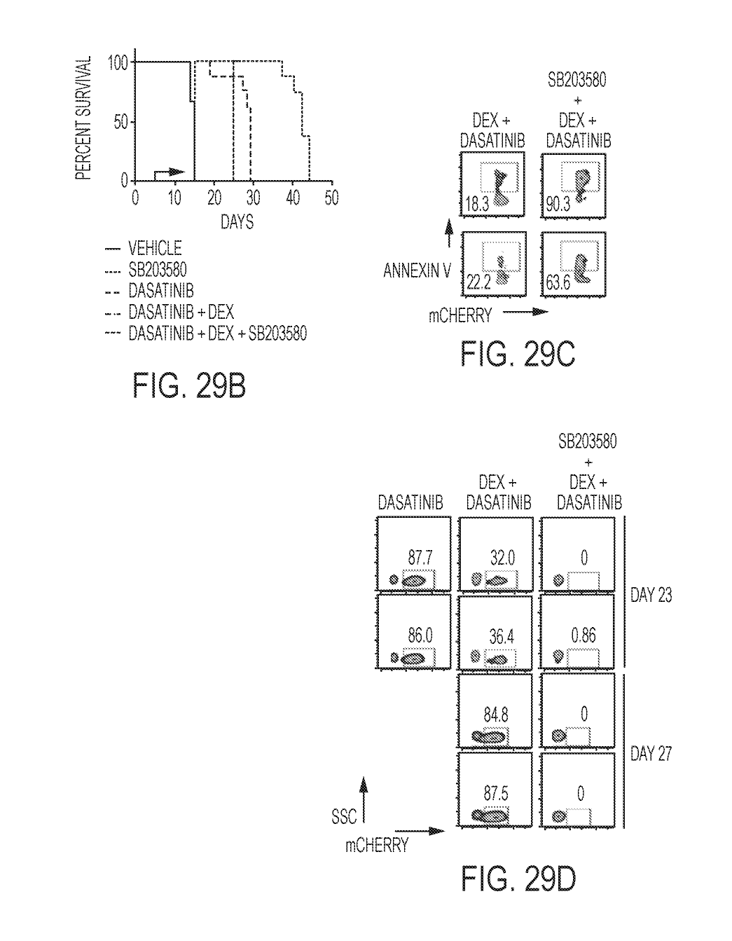

To test the efficacy of SB203580 in the treatment of BCR-ABL+ ALL in vivo, we transplanted NOD-SCID mice with mouse BCR-ABL+ ALL cells and then treated the mice with various combinations of dasatinib, dexamethasone, and SB203580. Dasatinib was used instead of imatinib because dasatinib has the same propensity as imatinib to induce leukemic cell cluster formation underneath MSCs in vitro and is more effective than imatinib in the treatment of BCR-ABL+ ALL in vivo (Boulos et al., 2011). Dexamethasone included because it was found that dexamethasone induced apoptosis in clustered leukemic cells in MSC/ALL cell co-culture. Leukemia progression was monitored by both bioluminescence imaging and quantifying mCherry+ ALL cells in peripheral blood samples by flow cytometry. Treatment with SB203580 alone did not slow leukemia progression or prolong survival compared with the vehicle control. As expected, mice treated with dasatinib or dasatinib+dexamethasone had significantly slower leukemia progression and longer survival than control mice. However, the therapeutic efficacy in mice treated with dasatinib+dexamethasone+SB203580 was significantly enhanced relative to all other treatment groups (P<0.0001) (FIG. 29A-B).

Consistent with these bioluminescence and survival analysis results, bone marrow leukemic cells from mice treated with dasatinib+dexamethasone+SB203580 showed higher rates of apoptosis than those from mice treated with dasatinib+dexamethasone (FIG. 29C). Moreover, mCherry+ leukemic cells were essentially absent in the peripheral blood of mice treated with dasatinib+dexamethasone+SB203580 at day 23 and 27 after transplantation, when mCherry+ leukemic cells were predominant and readily detectable in the mice treated with dasatinib+dexamethasone (FIG. 29D). These findings suggest that combining SB203580 with standard treatment could effectively prevent resistance to TKIs and improve outcomes of patients with BCR-ABL+ ALL.

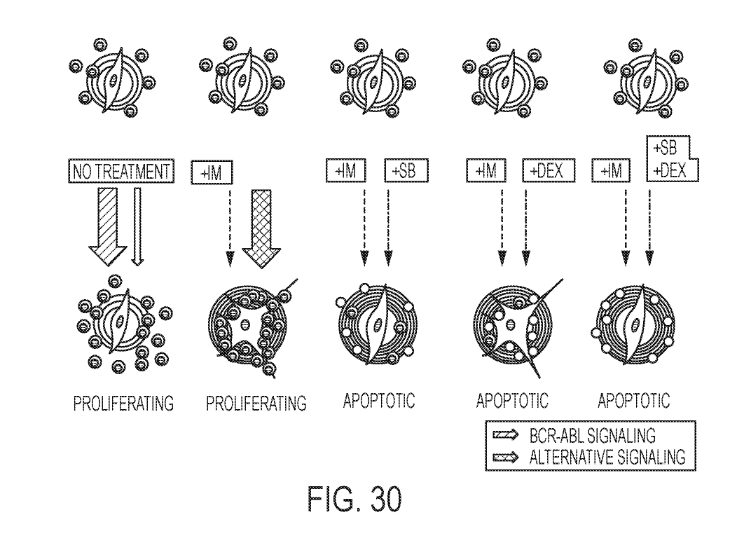

The in vivo findings demonstrated that combining SB203580 with potent BCR-ABL TKI dasatinib and dexamethasone substantially prevented TKI resistance and improved overall outcomes. Given these findings, a working model (FIG. 30) was proposed that illustrates the chain of signaling between MSCs and leukemic cells when BCR-ABL is inhibited and the consequences of the combination therapy with p38 MAPK inhibitor and dexamethasone. In conclusion, these findings suggest that combining p38 MAPK inhibitors with current induction and/or maintenance therapy would help eliminate the origin of imatinib resistance and yield promising outcomes for patients with BCR-ABL+ ALL.

### Example 6—Materials and Methods

Viral Vectors, Cell Culture, and Clinical Compound Screening:

Viral vectors and BCR-ABL+ mouse ALL cells were prepared as described previously (Mallampati et al., 2015). Briefly, for generating BCR-ABL+ mouse ALL cells, bone marrow derived progenitor-B cells were transduced with a p190 BCR-ABL-encoding virus which also co-expressed fluorescent reporter, mCherry. Later these transformed cells were labeled with Luciferase in a subsequent virus transduction. Culture conditions used for MSCs, leukemic cells, and co-culture of MSC/leukemic cells were also described previously (Mallampati et al., 2014).

The clinical compounds library was purchased from the John S. Dunn Gulf Coast Consortium for Chemical Genomics (Houston, Tex.). For clinical compounds screening experiments, OP9 cells (a mouse primary MSC line; ATCC, Manassas, Va.) were treated with imatinib (5 μM) and each of the clinical compounds from the library (6.6 μM for all compounds, except dexamethasone [50 nM]) for 3 days. Then, luciferase-labeled BCR-ABL+ mouse ALL cells were seeded on to the MSCs. Treatment was continued for 1 day, and the samples were evaluated via phase-contrast microscopy for the formation of leukemic cell clusters underneath the pretreated MSCs. To determine the compounds' toxicity in MSCs or leukemic cells, we examined the MSC/ALL cell co-cultures via microscopy and/or bioluminescence imaging.

For post-screening experiments, MSCs were treated with imatinib (5 μM) and/or SB203580 (20 μM) or dexamethasone (50 nM) for 4 days before ALL cells were seeded, and treatment continued for 1 day.

Microscopy:

Phase-contrast images of co-cultured MSCs and ALL cells were obtained using an Axio Observer.Z1 microscope, an AxioCam MR camera, and AxioVision software (Zeiss, Oberkochen, Germany). For screening, a cluster was defined as a group of more than five leukemic cells under the MSCs. The total number of leukemic cell clusters was the average number of clusters from three different fields (10× objective). For post-screening experiments, a cluster was defined as a group of more than 10 leukemic cells under the MSCs. The total number of leukemic cell clusters was the average number of clusters from 10 different fields (10× objective).

Bioluminescence Imaging of MSC/ALL Cell Co-Cultures:

To measure cell proliferation, co-cultures of MSCs and luciferase-labeled leukemic cells were mixed with D-Luciferin (Biosynth, Itasca, Ill.) to a final concentration of 0.5 mg/mL. Samples were incubated at room temperature for 1 minute and were imaged with the IVIS Lumina imaging system (PerkinElmer, Waltham, Mass.).

Patient Specimens:

Peripheral blood specimens were obtained from patients with BCR-ABL+ ALL leukemia (n=3) and leukemic cells were isolated by subjecting the samples to ACK red blood cell lysis buffer (Lonza, Basel, Switzerland). This study was approved by the Institutional Review Board at The University of Texas MD Anderson Cancer Center (Houston, Tex.) and was conducted in accordance with the Declaration of Helsinki. All patients provided written informed consent.

Apoptosis Assay:

Leukemic cell apoptosis was analyzed by flow cytometry using BD LSRFortessa or Accuri C6 flow cytometer (BD Biosciences) after the cells were stained with Annexin V (BD Biosciences, San Jose, Calif.). Data were analyzed by FlowJo software (Ashland, Oreg.).

Western Blot Analyses:

MSCs were subjected to lysis in the extraction buffer (Tris-HCl [pH 7.5, 50 mM]; sodium chloride [50 mM]; dithiothreitol [1 mM]; ethylenediaminetetraacetic acid [2 mM]; 1% NP-40; 0.1% sodium deoxycholate; and 0.1% sodium dodecyl sulfate) supplemented with protease and phosphatase inhibitors (Roche, Basel, Switzerland). Protein concentration in the lysates was measured by a protein assay reagent (Bio-Rad Laboratories, Hercules, Calif.). For Western blot analyses, lysates (35 μg) were denatured in sodium dodecyl sulfate Laemmli sample buffer with 5% beta-mercaptoethanol, were resolved via sodium dodecyl sulfate polyacrylamide gel electrophoresis, were blotted onto polyvinylidene difluoride membranes (Bio-Rad Laboratories), and were blocked with 5% nonfat milk powder dissolved in phosphate-buffered saline solution (PBS) with 0.2% Tween 20. The membranes were probed for phosphorylated PDGFR-α/β, total PDGFR-β, phosphorylated ATF2, total ATF2, and α-tubulin with the corresponding antibodies (1:1000 dilution in PBS with 0.2% Tween 20 and 1% nonfat milk powder; Cell Signaling Technology, Danvers, Mass.). The blots were then incubated with anti-rabbit secondary antibodies conjugated with horseradish peroxidase (1:3000 dilution in PBS with 0.2% Tween 20 and 1% nonfat milk powder; Sigma-Aldrich, St. Louis, Mo.), and bands were detected with a chemiluminescence detection system (Pierce Biotechnology, Rockford, Ill.).

Quantitative Real-Time Polymerase Chain Reaction Analysis:

Total RNA (100 ng) isolated from MSCs was reverse-transcribed with the SuperScript III First-Strand Synthesis System (Invitrogen, Carlsbad, Calif.) according to the manufacturer's instructions. Quantitative real-time polymerase chain reaction (PCR) analysis was performed using primers (200 nM) and SYBR Green PCR Master Mix (Applied Biosystems, Foster City, Calif.) on an ABI PRISM 7900HT Sequence Detection System (Thermo Fisher Scientific, Waltham, Mass.). Sequences of the gene specific primers used were detailed previously (Mallampati et al., 2015). Measurements were standardized to the expression of β-actin. Relative gene expression was calculated after normalizing to the expression levels in control cells, which was arbitrarily set to 1.

Animal Studies:

All mouse studies were reviewed and approved by the Institutional Animal Care and Use Committee of MD Anderson Cancer Center. Non-obese diabetic severe combined immunodeficient (NOD-SCID) mice were housed under high-barrier conditions in the Department of Veterinary Medicine and Surgery at MD Anderson. To generate the in vivo leukemia model, six to eight weeks old NOD-SCID mice were intravenously injected with luciferase- and mCherry-expressing BCR-ABL+ mouse leukemic cells (2×10⁶ cells); engraftment of the transplanted cells was confirmed by bioluminescence imaging. For bioluminescence imaging, each mouse was intraperitoneally injected with D-Luciferin (150 mg/kg) and imaged using the IVIS Lumina imaging system (PerkinElmer).

Leukemia treatment started 5 days after transplantation. Mice received oral dasatinib (dissolved in citric acid [80 mM]) at a dose of 10 mg per kg of body weight per day, 5 days a week. Dexamethasone was administered orally at a dose of 1 mg per kg of body weight per day and SB203580 (dissolved in 0.9% saline solution) was injected intraperitoneally at a dose of 40 mg per kg of body weight per day, 5 days a week. Treatment was continued until mice were succumbed to disease.

Bone marrow samples from the mice were harvested 4 days after initiating the drug treatment. One of the long bones from the hind leg was gently grounded and the bone marrow samples were collected and processed as described previously (Sun et al., 2013). Peripheral blood specimens were directly harvested from mice tail tips into PBS supplemented with ethylenediaminetetraacetic acid (2 mM). Red blood cells were subjected to lysis and were neutralized with 20% fetal bovine serum supplemented with alpha minimum essential medium. mCherry+ leukemic cells in the peripheral blood were detected by flow cytometry analysis after red blood cells were lysed.

Statistical Analysis:

For statistical comparisons between groups, student paired t-test was used and P value <0.05 was considered statistically significant. Kaplan-Meier survival analysis was performed with GraphPad Prism software (GraphPad Software, Inc, La Jolla, Calif.). P values were determined by log-rank test and P<0.0001 was considered statistically significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aggarwal R, Zhang T, Small E J, Armstrong A J. Neuroendocrine prostate cancer: subtypes, biology, and clinical outcomes. Journal of the National Comprehensive Cancer Network: JNCCN 2014; 12: 719-726.
Aparicio A, Tzelepi V, Araujo J C, Guo C C, Liang S, Troncoso P et al. Neuroendocrine prostate cancer xenografts with large-cell and small-cell features derived from a single patient's tumor: morphological, immunohistochemical, and gene expression profiles. Prostate 2011; 71: 846-856.
Aslakson C J, Miller F R. Selective events in the metastatic process defined by analysis of the sequential dissemination of subpopulations of a mouse mammary tumor. Cancer research 1992; 52: 1399-1405.
Burk U, Schubert J, Wellner U, Schmalhofer O, Vincan E, Spaderna S et al. A reciprocal repression between ZEB1 and members of the miR-200 family promotes EMT and invasion in cancer cells. EMBO reports 2008; 9: 582-589.
Castillo V, Valenzuela R, Huidobro C, Contreras H R, Castellon E A. Functional characteristics of cancer stem cells and their role in drug resistance of prostate cancer. Int J Oncol 2014; 45: 985-994.
Chaffer C L, Marjanovic N D, Lee T, Bell G, Kleer C G, Reinhardt F et al. Poised chromatin at the ZEB1 promoter enables breast cancer cell plasticity and enhances tumorigenicity. Cell 2013; 154: 61-74.
Cheriyan J, Webb A J, Sarov-Blat L, Elkhawad M, Wallace S M, Maki-Petaja K M et al. Inhibition of p38 mitogen-activated protein kinase improves nitric oxide-mediated vasodilatation and reduces inflammation in hypercholesterolemia. Circulation 2011; 123: 515-523.
Cohen S, Fleischmann R. Kinase inhibitors: a new approach to rheumatoid arthritis treatment. Curr Opin Rheumatol 2010; 22: 330-335.
Collins A T, Berry P A, Hyde C, Stower M J, Maitland N J. Prospective identification of tumorigenic prostate cancer stem cells. Cancer Res 2005; 65: 10946-10951.
Darnell J E, Jr. Transcription factors as targets for cancer therapy. Nature reviews Cancer 2002; 2: 740-749.
Daver N, Thomas D, Ravandi F, et al. Final report of a phase II study of imatinib mesylate with hyper-CVAD for the front-line treatment of adult patients with Philadelphia chromosome-positive acute lymphoblastic leukemia. Haematologica. 2015; 100(5):653-661.
Davies S P, Reddy H, Caivano M, Cohen P. Specificity and mechanism of action of some commonly used protein kinase inhibitors. The Biochemical journal 2000; 351: 95-105.
Davis J N, Wojno K J, Daignault S, Hofer M D, Kuefer R, Rubin M A et al. Elevated E2F1 inhibits transcription of the androgen receptor in metastatic hormone-resistant prostate cancer. Cancer Res 2006; 66: 11897-11906.
Eckert M A, Lwin T M, Chang A T, Kim J, Danis E, Ohno-Machado L et al. Twist1-induced invadopodia formation promotes tumor metastasis. Cancer cell 2011; 19: 372-386.
Elenbaas B, Spirio L, Koerner F, Fleming M D, Zimonjic D B, Donaher J L et al. Human breast cancer cells generated by oncogenic transformation of primary mammary epithelial cells. Genes & development 2001; 15: 50-65.
Fehm T, Muller V, Alix-Panabieres C, Pantel K. Micrometastatic spread in breast cancer: detection, molecular characterization and clinical relevance. Breast Cancer Res 2008; 10 Suppl 1: S1.
Gupta G P, Massague J. Cancer metastasis: building a framework. Cell 2006; 127: 679-695.
Hollier B G, Evans K, Mani S A. The epithelial-to-mesenchymal transition and cancer stem cells: a coalition against cancer therapies. J Mammary Gland Biol Neoplasia 2009; 14: 29-43.
Hollier B G, Tinnirello A A, Werden S J, Evans K W, Taube J H, Sarkar T R et al. FOXC2 expression links epithelial-mesenchymal transition and stem cell properties in breast cancer. Cancer research 2013; 73: 1981-1992.
Humphreys J M, Piala A T, Akella R, He H, Goldsmith E J. Precisely ordered phosphorylation reactions in the p38 mitogen-activated protein (MAP) kinase cascade. J Biol Chem. 2013; 288(32):23322-23330.
Hurt E M, Kawasaki B T, Klarmann G J, Thomas S B, Farrar W L. CD44+CD24(−) prostate cells are early cancer progenitor/stem cells that provide a model for patients with poor prognosis. Br J Cancer 2008; 98: 756-765.
Izumi K, Fang L Y, Mizokami A, Namiki M, Li L, Lin W J et al. Targeting the androgen receptor with siRNA promotes prostate cancer metastasis through enhanced macrophage recruitment via CCL2/CCR2-induced STAT3 activation. EMBO Mol Med 2013; 5: 1383-1401.
Kawano H, Kim S, Ohta K, Nakao T, Miyazaki H, Nakatani T et al. Differential contribution of three mitogen-activated protein kinases to PDGF-BB-induced mesangial cell proliferation and gene expression. Journal of the American Society of Nephrology: JASN 2003; 14: 584-592.
Koul H K, Pal M, Koul S. Role of p38 MAP Kinase Signal Transduction in Solid Tumors. Genes & cancer 2013; 4: 342-359.
Kumar S, Jiang M S, Adams J L, Lee J C. Pyridinylimidazole compound SB 203580 inhibits the activity but not the activation of p38 mitogen-activated protein kinase. Biochemical and biophysical research communications 1999; 263: 825-831.
Kume T. The Role of FoxC2 Transcription Factor in Tumor Angiogenesis. Journal of oncology 2012; 2012: 204593.
Li P, Wang J, Chu M, Zhang K, Yang R, Gao W Q. Zeb1 promotes androgen independence of prostate cancer via induction of stem cell-like properties. Exp Biol Med (Maywood) 2014; 239: 813-822.
Li P, Yang R, Gao W Q. Contributions of epithelial-mesenchymal transition and cancer stem cells to the development of castration resistance of prostate cancer. Mol Cancer 2014; 13: 55.
Mallampati S, Leng X, Ma H, et al. Tyrosine kinase inhibitors induce mesenchymal stem cell-mediated resistance in BCR-ABL+ acute lymphoblastic leukemia. Blood. 2015; 125(19):2968-2973.
Mallampati S, Sun B, Lu Y, et al. Integrated genetic approaches identify the molecular mechanisms of Sox4 in early B-cell development: intricate roles for RAG1/2 and CK1epsilon. Blood. 2014; 123(26):4064-4076.
Mani S A, Guo W, Liao M J, Eaton E N, Ayyanan A, Zhou A Y et al. The epithelial-mesenchymal transition generates cells with properties of stem cells. Cell 2008; 133: 704-715.
Mani S A, Yang J, Brooks M, Schwaninger G, Zhou A, Miura N et al. Mesenchyme Forkhead 1 (FOXC2) plays a key role in metastasis and is associated with aggressive basal-like breast cancers. Proceedings of the National Academy of Sciences of the United States of America 2007; 104: 10069-10074.
Niu Y, Altuwaijri S, Lai K P, Wu C T, Ricke W A, Messing E M et al. Androgen receptor is a tumor suppressor and proliferator in prostate cancer. Proc Natl Acad Sci USA 2008; 105: 12182-12187.
Obenauer J C, Cantley L C, Yaffe M B. Scansite 2.0: Proteome-wide prediction of cell signaling interactions using short sequence motifs. Nucleic acids research 2003; 31: 3635-3641.
Pantel K, Cote R J, Fodstad O. Detection and clinical importance of micrometastatic disease. J Natl Cancer Inst 1999; 91: 1113-1124.
Paranjape A N, Balaji S A, Mandal T, Krushik E V, Nagaraj P, Mukherjee G et al. Bmi1 regulates self-renewal and epithelial to mesenchymal transition in breast cancer cells through Nanog. BMC Cancer 2014; 14: 785.
Parimi V, Goyal R, Poropatich K, Yang X J. Neuroendocrine differentiation of prostate cancer: a review. American journal of clinical and experimental urology 2014; 2: 273-285.
Pulaski B A, Ostrand-Rosenberg S. Mouse 4T1 breast tumor model. Current protocols in immunology/edited by John E Coligan [et al] 2001; Chapter 20: Unit 20 22.
Pulukuri S M, Gondi C S, Lakka S S, Jutla A, Estes N, Gujrati M et al. RNA interference-directed knockdown of urokinase plasminogen activator and urokinase plasminogen activator receptor inhibits prostate cancer cell invasion, survival, and tumorigenicity in vivo. The Journal of biological chemistry 2005; 280: 36529-36540.
Qin J, Liu X, Laffin B, Chen X, Choy G, Jeter C R et al. The PSA(−/lo) prostate cancer cell population harbors self-renewing long-term tumor-propagating cells that resist castration. Cell Stem Cell 2012; 10: 556-569.
Santoni M, Conti A, Burattini L, Berardi R, Scarpelli M, Cheng L et al. Neuroendocrine differentiation in prostate cancer: novel morphological insights and future therapeutic perspectives. Biochimica et biophysica acta 2014; 1846: 630-637.
Sarkar T R, Battula V L, Werden S J, Vijay G V, Ramirez-Pena E Q, Taube J H et al. GD3 synthase regulates epithelial-mesenchymal transition and metastasis in breast cancer. Oncogene 2015; 34: 2958-2967.
Scher H I, Fizazi K, Saad F, Taplin M E, Sternberg C N, Miller K et al. Increased survival with enzalutamide in prostate cancer after chemotherapy. The New England journal of medicine 2012; 367: 1187-1197.
Shiota M, Zardan A, Takeuchi A, Kumano M, Beraldi E, Naito S et al. Clusterin mediates TGF-beta-induced epithelial-mesenchymal transition and metastasis via Twist1 in prostate cancer cells. Cancer Res 2012; 72: 5261-5272.
Stewart S A, Dykxhoorn D M, Palliser D, Mizuno H, Yu E Y, An D S et al. Lentivirus-delivered stable gene silencing by RNAi in primary cells. Rna 2003; 9: 493-501.
Sun B, Mallampati S, Gong Y, Wang D, Lefebvre V, Sun X. Sox4 is required for the survival of pro-B cells. J Immunol. 2013; 190(5):2080-2089.
Sun Y, Wang B E, Leong K G, Yue P, Li L, Jhunjhunwala S et al. Androgen deprivation causes epithelial-mesenchymal transition in the prostate: Implications for androgen-deprivation therapy. Cancer Research 2012; 72: 527-536.
Tamaki H, Harashima N, Hiraki M, Arichi N, Nishimura N, Shiina H et al. Bcl-2 family inhibition sensitizes human prostate cancer cells to docetaxel and promotes unexpected apoptosis under caspase-9 inhibition. Oncotarget 2014.
Taube J H, Herschkowitz J I, Komurov K, Zhou A Y, Gupta S, Yang J et al. Core epithelial-to-mesenchymal transition interactome gene-expression signature is associated with claudin-low and metaplastic breast cancer subtypes. Proceedings of the National Academy of Sciences of the United States of America 2010; 107: 15449-15454.
Taube J H, Malouf G G, Lu E, Sphyris N, Vijay V, Ramachandran P P et al. Epigenetic silencing of microRNA-203 is required for EMT and cancer stem cell properties. Scientific reports 2013; 3: 2687.
Tomlins S A, Mehra R, Rhodes D R, Cao X, Wang L, Dhanasekaran S M et al. Integrative molecular concept modeling of prostate cancer progression. Nat Genet 2007; 39: 41-51.
Tsai J H, Yang J. Epithelial-mesenchymal plasticity in carcinoma metastasis. Genes & development 2013; 27: 2192-2206.
Tzelepi V, Zhang J, Lu J F, Kleb B, Wu G, Wan X et al. Modeling a lethal prostate cancer variant with small-cell carcinoma features. Clin Cancer Res 2012; 18: 666-677.
Valastyan S, Weinberg R A. Tumor metastasis: molecular insights and evolving paradigms. Cell 2011; 147: 275-292.
Wellner U, Schubert J, Burk U C, Schmalhofer O, Zhu F, Sonntag A et al. The EMT-activator ZEB1 promotes tumorigenicity by repressing stemness-inhibiting microRNAs. Nat Cell Biol 2009; 11: 1487-1495.
Yan C, Grimm W A, Garner W L, Qin L, Travis T, Tan N et al. Epithelial to mesenchymal transition in human skin wound healing is induced by tumor necrosis factor-alpha through bone morphogenic protein-2. The American journal of pathology 2010; 176: 2247-2258.
Yoshizuka N, Chen R M, Xu Z, Liao R, Hong L, Hu W Y et al. A novel function of p38-regulated/activated kinase in endothelial cell migration and tumor angiogenesis. Molecular and cellular biology 2012; 32: 606-618.

1. A method of treating cancer in a subject comprising administering to the subject:
(a) a p38 MAPK inhibitor; and
(b) an anti-cancer therapy,
in an amount effective to treat, wherein the subject is identified as having cancer cells that express an elevated level of FOXC2 relative to a reference level.

2. The method of claim 1, wherein treating comprises inhibiting the growth of primary tumor cells, inhibiting the formation of metastases, inhibiting the growth of metastases, killing circulating cancer cells, inhibiting the growth and/or survival of cancer stem cells, inducing remission, extending remission, or inhibiting recurrence.

3. (canceled)

4. The method of claim 2, wherein the cancer stem cells have decreased expression of N-cadherin, collagen type III-α1, fibronectin, vimentin, Slug, Zeb1, or FOXC2 relative to expression prior to administration of the p38 MAPK inhibitor and the anti-cancer therapy.

5. (canceled)

6. The method of claim 1, wherein the cancer is prostate cancer, breast cancer, or leukemia.

7. The method of claim 1, wherein the prostate cancer is androgen-independent or castration-resistant prostate cancer.

8. (canceled)

9. The method of claim 1, wherein the subject has a decreased number of cancer stem cells relative to prior to administration of the p38 MAPK inhibitor and anti-cancer therapy.

10. The method of claim 9, wherein the cancer stem cells express one or more markers selected from a group consisting of ALDH, CD44, α2β1-integrin, Bmi1, and Sox2.

11. The method of claim 9, wherein the cancer stem cells do not express androgen receptor and/or prostate-specific antigen (PSA).

12. The method of claim 1, wherein the anti-cancer therapy is chemotherapy, radiotherapy, gene therapy, surgery, hormonal therapy, anti-angiogenic therapy or cytokine therapy.

13. The method of claim 12, wherein the hormonal therapy is an androgen-receptor inhibitor.

14. The method of claim 13, wherein the androgen-receptor inhibitor is Enzalutamide.

15. The method of claim 12, wherein the chemotherapy is Docetaxel.

16. The method of claim 1, wherein the p38 MAPK inhibitor is SB 203580, SB 203580 hydrochloride, SB681323 (Dilmapimod), LY2228820 dimesylate, BIRB 796 (Doramapimod), BMS-582949, Pamapimod, GW856553, ARRY-797AL 8697, AMG 548, CMPD-1, EO 1428, JX 401, RWJ 67657, TA 01, TA 02, VX 745, DBM 1285 dihydrochloride, ML 3403, SB 202190, SB 239063, SB 706504, SCIO 469 hydrochloride, SKF 86002 dihydrochloride, SX 011, TAK 715, VX 702, or PH-797804.

17-21. (canceled)

22. The method of claim 1, wherein the anti-cancer therapy and p38 MAPK inhibitor are administered essentially concomitantly or the anti-cancer therapy is administered before the p38 MAPK inhibitor.

23. (canceled)

24. The method of claim 1, wherein the anti-cancer therapy is administered after the p38 MAPK inhibitor.

25. (canceled)

26. The method of claim 1, further comprising administering at least one other anti-cancer therapy.

27. The method of claim 1, wherein more than one p38 MAPK inhibitor is administered.

28. The method of claim 1, wherein the cancer is resistant to chemotherapy or radiotherapy.

29-35. (canceled)

36. A method of treating breast cancer metastasis in a subject comprising administering to said subject a p38 mitogen activated protein kinase (MAPK) inhibitor in an amount effective to treat.

37-71. (canceled)

72. A method of treating a BCR-ABL related disorder in a subject comprising administering to said subject a p38 mitogen activated protein kinase (MAPK) inhibitor, a glucocorticoid receptor agonist, and a tyrosine kinase inhibitor in an amount effective to treat the disorder.

73-102. (canceled)


SEQUENCE LISTING (55 sequences)

SEQ ID NO: 1 - DNA, 21 bp, Artificial sequence
  Synthetic oligonucleotide
ccagtgcagcatgcgagcgat

SEQ ID NO: 2 - DNA, 21 bp, Artificial sequence
  Synthetic oligonucleotide
agaacatcatgaccctgcgaa

SEQ ID NO: 3 - DNA, 19 bp, Artificial sequence
  Synthetic oligonucleotide
taatttgtaacgttattgc

SEQ ID NO: 4 - DNA, 19 bp, Artificial sequence
  Synthetic oligonucleotide
tattctctatcttttgccg

SEQ ID NO: 5 - DNA, 19 bp, Artificial sequence
  Synthetic oligonucleotide
acttcttgacatctgagtg

SEQ ID NO: 6 - DNA, 19 bp, Artificial sequence
  Synthetic oligonucleotide
tgtggagcagggacattcg

SEQ ID NO: 7 - DNA, 20 bp, Artificial sequence
  Synthetic primer
gacgcttctaccagctcacc

SEQ ID NO: 8 - DNA, 20 bp, Artificial sequence
  Synthetic primer
gcttcactgggtgtggaaat

SEQ ID NO: 9 - DNA, 20 bp, Artificial sequence
  Synthetic primer
ccagggcttttcaaaaatga

SEQ ID NO: 10 - DNA, 20 bp, Artificial sequence
  Synthetic primer
ccgatccaatctgttctggt

SEQ ID NO: 11 - DNA, 20 bp, Artificial sequence
  Synthetic primer
tgcccagaaaatgaaaaagg

SEQ ID NO: 12 - DNA, 20 bp, Artificial sequence
  Synthetic primer
gtgtatgtggcaatgcgttc

SEQ ID NO: 13 - DNA, 20 bp, Artificial sequence
  Synthetic primer
gacaatgcccctcaagtgtt

SEQ ID NO: 14 - DNA, 20 bp, Artificial sequence
  Synthetic primer
ccattaagccgagtgatggt

SEQ ID NO: 15 - DNA, 20 bp, Artificial sequence
  Synthetic primer
gggaacaacttgatggtgct

SEQ ID NO: 16 - DNA, 20 bp, Artificial sequence
  Synthetic primer
cctccttcaacagcttcctg

SEQ ID NO: 17 - DNA, 20 bp, Artificial sequence
  Synthetic primer
cagtgggagacctcgagaag

SEQ ID NO: 18 - DNA, 20 bp, Artificial sequence
  Synthetic primer
gtccctcggaacatcagaaa

SEQ ID NO: 19 - DNA, 20 bp, Artificial sequence
  Synthetic primer
agaattactaccgggctgcg

SEQ ID NO: 20 - DNA, 20 bp, Artificial sequence
  Synthetic primer
tgagcgcgatgtagctgtag

SEQ ID NO: 21 - DNA, 20 bp, Artificial sequence
  Synthetic primer
tgctcgagatgtgatgaagg

SEQ ID NO: 22 - DNA, 20 bp, Artificial sequence
  Synthetic primer
tcccctgttgactggtcatt

SEQ ID NO: 23 - DNA, 20 bp, Artificial sequence
  Synthetic primer
agtgcgagaagcattcccaa

SEQ ID NO: 24 - DNA, 20 bp, Artificial sequence
  Synthetic primer
gaagctgtggctgacctgaa

SEQ ID NO: 25 - DNA, 20 bp, Artificial sequence
  Synthetic primer
gagcatttgcagacaggtca

SEQ ID NO: 26 - DNA, 20 bp, Artificial sequence
  Synthetic primer
gcttcggagtgaagaaatgc

SEQ ID NO: 27 - DNA, 20 bp, Artificial sequence
  Synthetic primer
accccacatccttctcactg

SEQ ID NO: 28 - DNA, 20 bp, Artificial sequence
  Synthetic primer
tacaaaaacccacgcagaca

SEQ ID NO: 29 - DNA, 20 bp, Artificial sequence
  Synthetic primer
ggagtccgcagtcttacgag

SEQ ID NO: 30 - DNA, 20 bp, Artificial sequence
  Synthetic primer
ccagcttgagggtctgaatc

SEQ ID NO: 31 - DNA, 20 bp, Artificial sequence
  Synthetic primer
gagaactttgccgttgaagc

SEQ ID NO: 32 - DNA, 20 bp, Artificial sequence
  Synthetic primer
tccagcagcttcctgtaggt

SEQ ID NO: 33 - DNA, 20 bp, Artificial sequence
  Synthetic primer
gcacaaccaagtgcagaaga

SEQ ID NO: 34 - DNA, 20 bp, Artificial sequence
  Synthetic primer
catttgcagattgaggctga

SEQ ID NO: 35 - DNA, 20 bp, Artificial sequence
  Synthetic primer
tgaaatgcatcgttgaggtc

SEQ ID NO: 36 - DNA, 20 bp, Artificial sequence
  Synthetic primer
accgctgtgtttcttctgct

SEQ ID NO: 37 - DNA, 20 bp, Artificial sequence
  Synthetic primer
taggacccaaggtggtcttg

SEQ ID NO: 38 - DNA, 20 bp, Artificial sequence
  Synthetic primer
tactctggagcccaccattc

SEQ ID NO: 39 - DNA, 20 bp, Artificial sequence
  Synthetic primer
cggcatttacaagtgtgtgg

SEQ ID NO: 40 - DNA, 20 bp, Artificial sequence
  Synthetic primer
gacatctcggcctttgtgtt

SEQ ID NO: 41 - DNA, 20 bp, Artificial sequence
  Synthetic primer
gtcccacgtgtcttccactt

SEQ ID NO: 42 - DNA, 20 bp, Artificial sequence
  Synthetic primer
cccaagtcaggccagtttta

SEQ ID NO: 43 - DNA, 36 bp, Artificial sequence
  Synthetic primer
cgagcggccccacggagcccctgagcgctctcaacc

SEQ ID NO: 44 - DNA, 36 bp, Artificial sequence; identical to SEQ ID NO: 43
  Synthetic primer

SEQ ID NO: 45 - DNA, 36 bp, Artificial sequence
  Synthetic primer
cgagcggccccacggcacccctgagcgctctcaacc

SEQ ID NO: 46 - DNA, 36 bp, Artificial sequence
  Synthetic primer
ggttgagagcgctcaggggtgccgtggggccgctcg

SEQ ID NO: 47 - DNA, 19 bp, Artificial sequence
  Synthetic oligonucleotide
ttcacagctagattactag

SEQ ID NO: 48 - DNA, 21 bp, Artificial sequence
  Synthetic oligonucleotide
cctgagcgagcagaattacta

SEQ ID NO: 49 - protein, 15 aa, Artificial sequence
  Synthetic peptide
HPSGPTSPLSALNLA

SEQ ID NO: 50 - protein, 15 aa, Artificial sequence
  Synthetic peptide
HPSGPGSPLGALNLA

SEQ ID NO: 51 - protein, 15 aa, Artificial sequence
  Synthetic peptide
HPSGPGSPLGALNLA

SEQ ID NO: 52 - protein, 15 aa, Artificial sequence
  Synthetic peptide
HPSGPTSPLSALNLA

SEQ ID NO: 53 - protein, 15 aa, Artificial sequence
  Synthetic peptide
HPSGPASPLSALNLA

SEQ ID NO: 54 - protein, 15 aa, Artificial sequence
  Synthetic peptide
HPSGPTSPLSALNLA

SEQ ID NO: 55 - protein, 15 aa, Artificial sequence
  Synthetic peptide
HPSGPTSPLSALNLA